(19) United States
(12) Patent Application Publication
(10) Pub. No.: US 2014/0107390 A1
(43) Pub. Date: Apr. 17, 2014

(54) IMPLEMENTATION AND EXPERIMENTAL RESULTS OF REAL-TIME 4D TUMOR TRACKING USING MULTI-LEAF COLLIMATOR (MLC), AND/OR MLC-CARRIAGE (MLC-BANK), AND/OR TREATMENT TABLE (COUCH)

(71) Applicants: Kevin Brown, West Sussex (GB); Tarun K. Podder, Greenville, NC (US); Yan Yu, Philadelphia, PA (US)

(72) Inventors: Kevin Brown, West Sussex (GB); Tarun K. Podder, Greenville, NC (US); Yan Yu, Philadelphia, PA (US)

(73) Assignees: ELEKTA AB (PUBL), Stockholm (SE); THOMAS JEFFERSON UNIVERSITY, Philadelphia, PA (US)

(21) Appl. No.: 13/650,910

(22) Filed: Oct. 12, 2012

(51) Int. Cl. A61N 5/10; G06F 19/00

(52) U.S. Cl. CPC A61N 5/107; A61N 5/1067; A61N 5/1068; G06F 19/3437
USPC 600/1; 703/11

(57) ABSTRACT

Methods and systems of operating a support structure and beam shaping mechanism in a manner that compensates for motion patterns exhibited by a patient, promotes comfort of the patient, and optimizes accuracy of delivery of radiotherapy to a targeted location within the patient. The support structure can be a treatment table or couch and the beam shaping mechanism can be a multi-leaf collimator (MLC), and/or an MLC-bank/-carriage. The control system can utilize algorithms for predicting tumor motion and loading condition on the table/couch during radiation therapy.

### TECHNICAL FIELD

The present invention generally relates to radiotherapy, specifically to methods and systems of operating a patient support structure and/or a radiotherapy delivery system, which may include a beam shaping mechanism, in a manner that compensates for motion patterns exhibited by a patient, promotes comfort of the patient, and optimizes accuracy of delivery of radiotherapy to a targeted location within the patient.

### SUMMARY OF THE INVENTION

The present invention is directed to a novel technique for four dimensional (4D) tumor tracking using a commercially available treatment couch that is commonly used in clinics. Implementation strategies are discussed and experimental results including evaluation of tumor tracking accuracies in a clinical setting are presented.

Patient support systems such as couches and tables are capable of positioning patients accurately; however, current devices and methods either do not address or do not adequately compensate for tumor movement in the thoracic region caused by respiratory and cardiac motions. Implementation of a real-time tracking control technique is presented together with experimental results in tumor motion compensation in four dimensions (superior-inferior, lateral, anterior-posterior, and time). A novel control system for the treatment couch was developed and implemented. The primary design specifications for the implementation of the novel technique were: a) the treatment couch should maintain all previous/normal features for patient setup and positioning, b) the new control system could be used as a parallel system when tumor tracking was clinically desired, and c) tracking could be performed in a single direction and/or concurrently in all three directions of the couch motion (longitudinal, lateral and vertical). The implementation of such robotic technique to a regular patient support system for tumor tracking has not been reported so far. To evaluate the performance of such a robotic couch, we investigated the mechanical characteristics of the system including system positioning resolution, repeatability, accuracy, and tracking performance. Furthermore, by measuring radiation dose delivered from a linear accelerator (Linac) in conjunction with robotic couch tracking, the dosimetric properties of using the proposed system were tested. To investigate the accuracy of real-time tracking in the clinical setting, existing clinically used treatment couch/table was replaced with experimental couch/table of the present invention while the linear accelerator was used to deliver the treatment plans with and without tracking. The results of radiation dose distribution from these two sets of experiments were compared and are presented here.

Under test, mechanical accuracies were 0.12, 0.14, and 0.18 mm in all three directions. The repeatability of the desired motion in the range of 50 mm was within ±0.2 mm. The differences of central axis dose between the three-dimensional conformal radiation therapy (3D-CRT) stationary plan and two tracking plans with different motion trajectories were 0.21% and 1.19%. The absolute dose differences of both 3D tracking plans comparing to the stationary plan were 1.09% and 1.20%. Comparing the stationary intensity modulated radiation therapy (IMRT) plan with the tracking plan, it was observed that the central axis dose difference was −0.87% and the absolute difference of both plans was 0.55%.

The experimental results show that the treatment tables of the present invention can be effectively used for real-time tumor tracking with a high level of accuracy. It was determined that 4D tumor tracking was feasible using the system of the present invention comprising the robotic or tracking couch, appropriate tracking methodologies and appropriate implementations in control systems.

Some 226,000 new cases of lung cancer are expected in 2012, accounting for 14% of all cancer diagnoses. Lung cancer causes more deaths than any other cancers in both men and women. More than 160,000 deaths, accounting for about 28% of all cancer deaths, are expected to occur in 2012 (American Cancer Society Cancer Facts & Figure 2012). (ACC Website, American Cancer Society Cancer Facts and Figure 2012: http://www.cancer.org/Research/CancerFactsFigures/index, accessed in March 2012.)

Cancer in the lung and other organs in the thoracic and abdominal regions can move up to 2-3 cm or more during breathing cycle and cardiac motion. (H. Shirato, K. Suzuki, G. C. Sharp, K. Fujita, R. Onimaru, M. Fujino, N. Kato, Y. Osaka, R. Kinoshita, H. Taguchi, S. Onodera, K. Miyasaka, “Speed and amplitude of lung tumor motion precisely detected in four-dimensional setup and in real-time tumor-tracking radiotherapy”, Int. J. Radiat. Oncol. Biol. Phys. 64, 1229-1236 (2006); C. Ozhasoglu, M. J. Murphy, “Issues in respiratory motion compensation during external-beam radiotherapy”, Int. J. Radiat. Oncol. Biol. Phys. 52, 1389-1399 (2002); and P. J. Keall, G. S. Mageras, J. M. Baiter, R. S. Emery, K. M. Forster, S. B. Jiang, J. M. Kapatoes, D. A. Low, M. J. Murphy, B. R. Murray, C. R. Ramsey, M. B. Van Herk, S. S. Vedam, J. W. Wong, E. Yorke, “The management of respiratory motion in radiation oncology report of AAPM task group 76”, Med. Phys. 33, 3874-3900 (2006).) Nowadays, patients treated for lung cancers, especially for early-stage lung cancers, are surviving longer. Therefore, intrafraction (that is, during the time a daily fraction of radiation dose is being delivered by the Linac) motion management and related treatment margins are becoming increasingly important in the context of sparing healthy tissues and adjacent critical structures. This requires concurrent irradiation of the whole tumor volume while at the same time avoiding unnecessary irradiation to adjacent noncancerous tissues that would move into the radiation beam in the absence of compensating for tumor movement.

Recently, the scientific community has devoted much investigation into various aspects of tumor motion management and the development of tools to deliver radiation dose to moving targets. The following studies on tumor tracking have been published in the past decade:
T. K. Podder, I. Buzurovic, Y. Hu, J. M. Galvin, Y. Yu, “Partial transmission high-speed continuous tracking multi-leaf collimator for 4D adaptive radiation therapy”, Proc. of IEEE Int. Conf. on Bioinformatics and Bioeng., 1108-1112 (2007).
T. Depuydt, D. Verellen, O. Haas, T. Gevaert, N. Linthout, M. Duchateau, K. Tournel, T. Reynders, K. Leysen, M. Hoogeman, G. Storme, M. D. Ridder, “Geometric accuracy of a novel gimbals based radiation therapy tumor tracking system”, Radiotherapy and Oncol. 98, 365-372 (2011).
M. Falk, P. M. of Rosenschold, P. Keall, H. Cattell, B. C. Cho, P. Poulsen, S. Povzner, A. Sawant, J. Zimmerman, S. Korreman S, “Real-time dynamic MLC tracking for inversely optimized arc radiotherapy”, Radiotherapy and Oncol. 94, 218-223 (2010).
A. Krauss, S, Nill, M. Tacke, U. Oelfke, “Electromagnetic real-time tumor position monitoring and dynamic multileaf collimator tracking using a Siemens 160 MLC: Geometric and dosimetric accuracy of an integrated system”, Int. J. Radiat. Oncol. Biol. Phys. 79, 579-587 (2011).
P. R. Poulsen, B. Cho, A. Sawant, D. Ruan, P. J. Keall, “Detailed analysis of latencies in image-based dynamic MLC racking”, Med. Phys. 37, 4998-5005 (2010).
P. R. Poulsen, B. Cho, A. Sawant, D. Ruan, P. J. Keall, “Dynamic MLC tracking of moving targets with a single kV imager for 3D conformal and IMRT treatments”, Acta Oncol. 49, 1092-1100 (2010).
J. Zimmerman, S. Korreman, G. Persson, H. Cattell, M. Svatos, A. Sawant, R. Venkat, D. Carlson, P. Keall, “DMLC motion tracking of moving targets for intensity modulated arc therapy treatment—A feasibility study”, Acta Oncol. 48, 245-250 (2009).
T. Lin, L. I. Cervĩo, X. Tang, N. Vasconcelos, S. B. Jiang, “Fluoroscopic tumor tracking for image-guided lung cancer radiotherapy”, Phys. Med. Biol. 54, 981-992 (2009).
M. Riboldi, G. C. Sharp, G. Baroni, G. T. Y. Chen, “Four-dimensional targeting error analysis in image-guided radiotherapy”, Phys. Med. Biol. 54, 5995-6008 (2009).
N. Riaz, P. Shanker, R. Wiersma, O. Gudmundsson, W. Mao, B. Widrow, L. Xing, “Predicting respiratory tumor motion with multi-dimensional adaptive filters and support vector regression”, Phys. Med. Biol. 54, 5735-5748 (2009).
K. Huang, I. Buzurovic, Y. Yu, T. K. Podder, “A Comparative Study of a Novel AE-nLMS Filter and Two Traditional Filters in Predicting Respiration Induced Motion of the Tumor”, Proc. of IEEE Int. Conf. on Bioinformatics and Bioeng., 281-282 (2010).
J. Rottmann, M. Aristophanous, A. Chen, L. Court, R. Berbeco, “A multi-region algorithm for markerless beam's-eye view lung tumor tracking”, Phys. Med. Biol. 55, 5585-5598 (2010).
B. Cho, P. R. Poulsen, P. J. Keall, “Real-time tumor tracking using sequential kV imaging combined with respiratory monitoring: A general framework applicable to commonly used IGRT systems”, Phys. Med. Biol. 55, 3299-3316 (2010).
J. H. Lewis, R. Li, W. T. Watkins, J. D. Lawson, W. P. Segars, L. I. Cervĩo, W. Y. Song, S. B. Jiang, “Markerless lung tumor tracking and trajectory reconstruction using rotational cone-beam projections: A feasibility study”, Phys. Med. Biol. 55, 2505-2522 (2010).
W. D. D'Souza, T. J. McAvoy, “An analysis of the treatment couch and control system dynamics for respiration-induced motion compensation”, Med. Phys. 33, 4701-4709 (2006).
T. Podder, I. Buzurovic, Y. Yu, “Coordinated dynamics-based control of robotic couch and MLC-bank for feedforward radiation therapy”, Int. J. Comp.—Assis. Rad. Surg. 2, 49-52 (2007).
D. Putra, P. Skworcow, O. C. L. Haas, K. J. Burnham, J. A. Mills, “Output-feedback tracking for tumour motion compensation in adaptive radiotherapy”, Proc. IEEE of American Control Conf., 3414-3419 (2007).
I. Buzurovic, K. Huang, Y. Yu, T. K. Podder, “Tumor Motion Prediction and Tracking in Adaptive Radiotherapy”, Proc. of IEEE Int. Conf. on Bioinformatics and Bioeng. 273-278 (2010).
I. Buzurovic, K. Huang, Y. Yu, T. K. Podder, “A robotic approach to 4D real-time tumor tracking for radiotherapy”, Phys. Med. Biol. 56, 1299-1318 (2011).
T. K Podder, I. Buzurovic, J. M. Galvin, Y. Yu, “Dynamics-based decentralized control of robotic couch and multi-leaf collimators for tracking tumor motion” Proc. of IEEE Int. Conf. on Robotics and Automat., 2496-2502 (2008).
I. Buzurovic, Y. Yu, T. K. Podder, “Active Tracking and Dynamic Dose Delivery for Robotic Couch in Radiation Therapy”, Proc. of IEEE Int. Conf. on Eng. in Medicine and Biol., 2156-2159 (2011).
W. D. D'Souza, K. T. Malinowski, S. Van Liew, G. D'Souza, K. Asbury, T. J. McAvoy, M. M. Suntharalingam, W. F. Regine, “Investigation of motion sickness and inertial stability on a moving couch for intra-fraction motion compensation”, Acta Oncol. 48, 1198-1203 (2009).
R. A. Sweeney, W. Arnold, E. Steixner, M. Nevinny-Stickel, P. Lukas, “Compensating for tumor motion by a 6-degree-of-freedom treatment couch: Is patient tolerance an issue?”, Int. J. Radiat. Oncol. Biol. Phys. 74, 168-171 (2009).
J. Wilbert, K. Baier, A. Richter, C. Herrmann, L. Ma, M. Flentje, M. Guckenberger, “Influence of continuous table motion on patient breathing patterns”, Int. J. Radiat. Oncol. Biol. Phys. 77, 622-629 (2010).
A. Harsolia, G. D. Hugo, L. L. Kestin, I. S. Grills, D. Yan, “Dosimetric advantages of four-dimensional adaptive image-guided radiotherapy for lung tumors using online cone-beam computed tomography”, Int. J. Radiat. Oncol. Biol. Phys. 70, 582-589 (2008).
I. Buzurovic, M. Werner-Wasik, T. Biswas, J. Galvin, A. P. Dicker, Y. Yu, T. Podder, “Dosimetric Advantages of Active Tracking and Dynamic Delivery”, Med. Phys. 37, 3191 (2010).
I. Buzurovic, K. Huang, M. Werner-Wasik, T. Biswas, A. P. Dicker, J. Galvin, Y. Yu, T. Podder, “Dosimetric Evaluation of Tumor Tracking in 4D Radiotherapy”, Int. J. Radiat. Oncol. Biol. Phys. 78, 5689 (2010).

Several methods are currently available for monitoring and controlling or compensating respiratory motion during radiation therapy. These methods are: slow CT scanning, inhale and exhale breath-hold CT imaging, or 4D CT/respiration-correlated CT, gating using an external respiration signal, gating using internal fiducial markers. Breath-holding methods include deep-inspiration breath-hold, active-breathing control, self-held breath-hold without respiratory monitoring, and forced shallow breathing with the assistance of abdominal compression using an external (such as mechanical) device.

It is also possible to employ real-time tumor tracking to compensate for tumor movement. However, none of these methods is perfect; different methods have different types of drawbacks. For example, conventional imaging and planning cannot be done in real-time in a strict sense, 4D CT imaging requires adequate respiratory motion patterns; the respiratory gating technique suffers from severely truncated duty-cycle of radiation delivery; breath-hold method requires the patient to be trained (uncomfortable, particularly for patients with compromised pulmonary capacity), hypo-oxygenation due to breath-hold may reduce the effectiveness of the killing of cancerous cells; shallow-breathing with abdominal compression approach is uncomfortable for the patient and may also affect tumor oxygenation. (P. J. Keall, G. S. Mageras, J. M. Balter, R. S. Emery, K. M. Forster, S. B. Jiang, J. M. Kapatoes, D. A. Low, M. J. Murphy, B. R. Murray, C. R. Ramsey, M. B. Van Herk, S. S. Vedam, J. W. Wong, E. Yorke, “The management of respiratory motion in radiation oncology report of AAPM task group 76”, Med. Phys. 33, 3874-3900 (2006).) Although real-time tracking promises better results, it is more involved because of its rudimentary stage of development. Apart from the traditional methods for tumor motion compensation, such as breath-hold and gating, other scientific investigations involve real-time tumor motion compensation and dynamic delivery of radiation dose.

Real-time tumor tracking, sometimes called Active Tracking and Dynamic Delivery (ATDD) (T. K. Podder, I. Buzurovic, Y. Hu, Galvin J. M., Y. Yu, “Partial transmission high-speed continuous tracking multi-leaf collimator for 4D adaptive radiation therapy”, Proc. of IEEE Int. Conf. on Bioinformatics and Bioeng., 1108-1112 (2007); T. Podder, I. Buzurovic, Y. Yu, “Coordinated dynamics-based control of robotic couch and MLC-bank for feedforward radiation therapy”, Int. J. Comp.—Assis. Rad. Surg. 2, 49-52 (2007); I. Buzurovic, K. Huang, Y. Yu, T. K. Podder, “Tumor Motion Prediction and Tracking in Adaptive Radiotherapy”, Proc. of IEEE Int. Conf. on Bioinformatics and Bioeng. 273-278 (2010); I. Buzurovic, K. Huang, Y. Yu, T. K. Podder, “A robotic approach to 4D real-time tumor tracking for radiotherapy”, Phys. Med. Biol. 56, 1299-1318 (2011); T. K Podder, I. Buzurovic, J. M. Galvin, Y. Yu, “Dynamics-based decentralized control of robotic couch and multi-leaf collimators for tracking tumor motion” Proc. of IEEE Int. Conf. on Robotics and Automat., 2496-2502 (2008); I. Buzurovic, Y. Yu, T. K. Podder, “Active Tracking and Dynamic Dose Delivery for Robotic Couch in Radiation Therapy”, Proc. of IEEE Int. Conf. on Eng. in Medicine and Biol., 2156-2159 (2011)), can be accomplished in three different ways: (a) adjusting the multileaf collimator (MLC) and/or MLC-carriage, (b) adjusting the couch, and (c) adjusting the MLC (and/or MLC-carriage) and the couch simultaneously. Measurements of the accuracy of real-time dynamic multileaf collimator (DMLC) tracking attracted significant attention. (P. R. Poulsen, B. Cho, A. Sawant, D. Ruan, P. J. Keall, “Detailed analysis of latencies in image-based dynamic MLC racking”, Med. Phys. 37, 4998-5005 (2010); P. R. Poulsen, B. Cho, A. Sawant, D. Ruan, P. J. Keall, “Dynamic MLC tracking of moving targets with a single kV imager for 3D conformal and IMRT treatments”, Acta Oncol. 49, 1092-1100 (2010); J. Zimmerman, S. Korreman, G. Persson, H. Cattell, M. Svatos, A. Sawant, R. Venkat, D. Carlson, P. Keall, “DMLC motion tracking of moving targets for intensity modulated arc therapy treatment—A feasibility study”, Acta Oncol. 48, 245-250 (2009).) Preliminary work on tumor motion compensation using a robotic couch was reported by several research groups. (W. D. D'Souza, T. J. McAvoy, “An analysis of the treatment couch and control system dynamics for respiration-induced motion compensation”, Med. Phys. 33, 4701-4709 (2006); T. Podder, I. Buzurovic, Y. Yu, “Coordinated dynamics-based control of robotic couch and MLC-bank for feedforward radiation therapy”, Int. J. Comp.—Assis. Rad. Surg. 2, 49-52 (2007); D. Putra, P. Skworcow, O. C. L. Haas, K. J. Burnham, J. A. Mills, “Output-feedback tracking for tumour motion compensation in adaptive radiotherapy”, Proc. IEEE of American Control Conf., 3414-3419 (2007); I. Buzurovic, K. Huang, Y. Yu, T. K. Podder, “Tumor Motion Prediction and Tracking in Adaptive Radiotherapy”, Proc. of IEEE Int. Conf. on Bioinformatics and Bioeng. 273-278 (2010).) In this approach, the robotic treatment couch/table moves during delivery of the radiation beam and compensating for breathing-induced tumor motion. D'Souza et al. performed an analysis of the couch dynamics and control systems in order to provide an estimate of the design specifications that would be required for effective motion compensation of respiration induced lung and abdominal tumors exhibiting motion displacements of up to 3 cm using the treatment couch. (W. D. D'Souza, T. J. McAvoy, “An analysis of the treatment couch and control system dynamics for respiration-induced motion compensation”, Med. Phys. 33, 4701-4709 (2006).) Furthermore, the tumor motion trajectory was decomposed and allocated to the subsystems (MLC-bank/-carriage and robotic couch) based on their natural frequency domains using a wavelet technique. (T. Podder, I. Buzurovic, Y. Yu, “Coordinated dynamics-based control of robotic couch and MLC-bank for feedforward radiation therapy”, Int. J. Comp.—Assis. Rad. Surg. 2, 49-52 (2007).) Putra et al. considered a compensation strategy for tumor motion caused by respiration and patient movements during radiotherapy treatments using a controlled patient support system (PSS) and an output-feedback model with a predictive control scheme. (D. Putra, P. Skworcow, O. C. L. Haas, K. J. Burnham, J. A. Mills, “Output-feedback tracking for tumour motion compensation in adaptive radiotherapy”, Proc. IEEE of American Control Conf., 3414-3419 (2007).) Detailed dynamic-based control scheme with a prediction module for commercially available treatment couches was presented. (I. Buzurovic, K. Huang, Y. Yu, T. K. Podder, “Tumor Motion Prediction and Tracking in Adaptive Radiotherapy”, Proc. of IEEE Int. Conf. on Bioinformatics and Bioeng. 273-278 (2010); I. Buzurovic, K. Huang, Y. Yu, T. K. Podder, “A robotic approach to 4D real-time tumor tracking for radiotherapy”, Phys. Med. Biol. 56, 1299-1318 (2011).) In these studies, a prediction module was developed to predict tumor motion and to compensate errors due to the delay in the system response.

The simultaneous usage of MLC and couch for tumor motion compensation was presented in several publications. (T. K. Podder, I. Buzurovic, Y. Hu, Galvin J. M., Y. Yu, “Partial transmission high-speed continuous tracking multi-leaf collimator for 4D adaptive radiation therapy”, Proc. of IEEE Int. Conf. on Bioinformatics and Bioeng., 1108-1112 (2007); T. Podder, I. Buzurovic, Y. Yu, “Coordinated dynamics-based control of robotic couch and MLC-bank for feedforward radiation therapy”, Int. J. Comp.—Assis. Rad. Surg. 2, 49-52 (2007); T. K. Podder, I. Buzurovic, J. M. Galvin, Y. Yu, “Dynamics-based decentralized control of robotic couch and multi-leaf collimators for tracking tumor motion” Proc. of IEEE Int. Conf. on Robotics and Automat., 2496-2502 (2008).) During real-time tracking several parameters of the control system, such as patient mass and breathing pattern, are initially uncertain and may vary during the long course of treatment. To solve these problems, feed-forward adaptive control was adopted to minimize irradiation to the healthy tissue and spare critical organs. (I. Buzurovic, Y. Yu, T. K. Podder, “Active Tracking and Dynamic Dose Delivery for Robotic Couch in Radiation Therapy”, Proc. of IEEE In Conf. on Eng. in Medicine and Biol., 2156-2159 (2011).)

Implementation of the couch motion for tumor motion compensation may pose additional problems or discomfort to patients under treatment. Several studies have addressed and investigated whether patients could tolerate the motion of the treatment couch that would compensate for the breathing-induced tumor motion. (W. D. D'Souza, K. T. Malinowski, S. Van Liew, G. D'Souza, K. Asbury, T. J. McAvoy, M. M. Suntharalingam, W. F. Regine, “Investigation of motion sickness and inertial stability on a moving couch for intra-fraction motion compensation”, Acta Oncol. 48, 1198-1203 (2009); R. A. Sweeney, W. Arnold, E. Steixner, M. Nevinny-Stickel, P. Lukas, “Compensating for tumor motion by a 6-degree-of-freedom treatment couch: Is patient tolerance an issue?”, Int. J. Radiat. Oncol. Biol. Phys. 74, 168-171 (2009); J. Wilbert, K. Baier, A. Richter, C. Herrmann, L. Ma, M. Flentje, M. Guckenberger, “Influence of continuous table motion on patient breathing patterns”, Int. J. Radiat. Oncol. Biol. Phys. 77, 622-629 (2010).) Among 4,800 responses, the results show that the patients do not suffer from motion sickness or external surface instability on a moving couch. (W. D. D'Souza, K. T. Malinowski, S. Van Liew, G. D'Souza, K. Asbury, T. J. McAvoy, M. M. Suntharalingam, W. F. Regine, “Investigation of motion sickness and inertial stability on a moving couch for intra-fraction motion compensation”, Acta Oncol. 48, 1198-1203 (2009).) Sweeney et al. concluded that the patients tolerated the compensatory couch motion, and motion sickness should not pose a problem in the investigation of different tumor tracking methods. (R. A. Sweeney, W. Arnold, E. Steixner, M. Nevinny-Stickel, P. Lukas, “Compensating for tumor motion by a 6-degree-of-freedom treatment couch: Is patient tolerance an issue?”, Int. J. Radiat. Oncol. Biol. Phys. 74, 168-171 (2009).) The influence of continuous table motions on patient breathing patterns for the compensation of moving targets by a robotic treatment couch was investigated and was found that the continuous table motion was well tolerated by all test persons. (J. Wilbert, K. Baier, A. Richter, C. Herrmann, L. Ma, M. Flentje, M. Guckenberger, “Influence of continuous table motion on patient breathing patterns”, Int. J. Radiat. Oncol. Biol. Phys. 77, 622-629 (2010).) The small changes observed in breathing patterns support the application of motion compensation by a robotic treatment couch. Several researchers reported dosimetric justification and potential advantages of tumor tracking. (A. Harsolia, G. D. Hugo, L. L. Kestin, I. S. Grills, D. Yan, “Dosimetric advantages of four-dimensional adaptive image-guided radiotherapy for lung tumors using online cone-beam computed tomography”, Int. J. Radiat. Oncol. Biol. Phys. 70, 582-589 (2008); I. Buzurovic, M. Werner-Wasik, T. Biswas, J. Galvin, A. P. Dicker, Y. Yu, T. Podder, “Dosimetric Advantages of Active Tracking and Dynamic Delivery”, Med. Phys. 37, 3191 (2010); I. Buzurovic, K. Huang, M. Werner-Wasik, T. Biswas, A. P. Dicker, J. Galvin, Y. Yu, T. Podder, “Dosimetric Evaluation of Tumor Tracking in 4D Radiotherapy”, Int. J. Radiat. Oncol. Biol. Phys. 78, 5689 (2010).)

Based on these published research and clinical investigations, the importance of developing tracking techniques is well established. Implementation of real-time tracking techniques can minimize irradiation to healthy tissues and improves sparing of critical organs. Consequently, quality of patient treatment can be improved and potential reduction in secondary occurrence of cancer is possible.

In the present specification, an adaptation of a commercial treatment couch for the simultaneous tracking in all three directions (patient's superior-inferior, medial-lateral, and anterior-posterior) has been proposed. In the following part, the novel control methodology necessary for real-time tracking of moving tumor was presented. A brief description of the system integration for tracking tasks has been provided. To evaluate the system performances several tests have been performed, such as: couch performance tests, mechanical tests, and dosimetry tests of tumor tracking using the external radiation beam.

### BRIEF DESCRIPTION OF THE DRAWINGS

The accompanying drawings, which are incorporated into this specification, illustrate one or more exemplary embodiments of the inventions disclosed herein and, together with the detailed description, serve to explain the principles and exemplary implementations of these inventions. One of skill in the art will understand that the drawings are illustrative only, and that what is depicted therein may be adapted based on the text of the specification and the spirit and scope of the teachings herein.

In the drawings, where like reference numerals refer to like reference in the specification:

FIG. 1 depicts closed-loop control of a robotic couch for compensating respiratory motion of a tumor;

FIG. 2 depicts a preliminary schematic of a decentralized coordinated dynamics-based closed-loop controller for a couch and an MLC (MLC-bank/-carriage);

FIG. 3 is a schematic of Adaptive Filter Training;

FIG. 4 is a schematic of the Factor Determination Process for AE Filters;

FIG. 5 is a chart of Normal and Irregular Respiration Signals;

FIGS. 6(a)-(b) are charts of velocities predicted by the adaptive nLMS filter in the acceleration-enhanced method for (FIG. 6(a)) normal respiration at 125 ms, and for (FIG. 6(b)) irregular respiration at 250 ms;

FIGS. 7(a)-(c) are charts of positions predicted by the adaptive ANN, nLMS, AE-nLMS, and AE-ANN filters in normal respiration, and the close-ups of the dotted boxes in the left panels at the times of (FIG. 7(a)) 125 ms, (FIG. 7(b)) 187.5 ms, and (FIG. 7(c)) 250 ms;

FIGS. 8(a)-(b) are charts of positions predicted by the adaptive ANN, nLMS, AE-nLMS, and AE-ANN filters from the real position in the irregular respiration, as well as the close-ups of the dotted boxes at the times of (FIG. 8(a)) 156 ms, and (FIG. 8(b)) 250 ms;

FIG. 9 is a chart of DVH for phase 10 without tumor motion compensation or prediction (one of the representative patient case);

FIG. 10 is a chart of DVH with tumor motion compensation (one of the representative patient case);

FIGS. 11(a)-(c) are histograms of the residual motion of the tumor with and without tracking (prediction) on (FIG. 11(a)) X direction, (FIG. 11(b)) Y direction, and (FIG. 11(c)) Z direction;

FIG. 12 includes charts of tumor centroid displacement due to cardiac and/or respiratory motion (patient data) Xₘₒₜ and output of the prediction module X_{d} in X, Y and Z directions—real time data;

FIG. 13 is a chart of tumor tracking errors; FIG. 13(a) depicts εₓ, ε_{y}, ε_{z} for control system in X, Y and Z directions for ELEKTA Precise Table™; FIG. 13(b) depicts the error amplitudes for steady states;

FIG. 14 includes charts of overall system error in X, Y and Z directions for ELEKTA Precise Table™;

FIG. 15 includes charts of Velocities in X, Y and Z directions for ELEKTA Precise Table™;

FIG. 16 includes charts of actuation of the ELEKTA Precise Table™ during tumor tracking;

FIG. 17 is a chart of PTV and CTV coverage was not compromised during the tumor tracking procedure (representative case);

FIG. 18 is a schematic view of ELEKTA Precise Table™; FIG. 18(a)-(b) are internal isometric views and FIG. 18(c) is a system model; FIG. 18(d) depicts functional elements of tumor tracking control system.

FIG. 19(a) is a chart of the motion of the decomposed tumor centroid, FIG. 19(b) is a chart of the motion of the table and FIG. 19(c) is a chart of the motion of the tumor relative motions in absolute coordinate system in the tracking model;

FIG. 20(a) depicts control system integration parts, and FIG. 20(b) depicts ELEKTA Precise Table™ robotic treatment couch—experimental setup with reference coordinate system; FIG. 20(c) depicts installation of the encoder to vertical lift motor; FIG. 20(d) depicts installation of the encoder for longitudinal couch motion (X direction).

FIG. 21(a) depicts an experimental setup with a Sun Nuclear programmable 4D phantom on the top of the table, and FIG. 21(b) depicts the metal plate with the hole fixed on the top of the 4D phantom;

FIGS. 22(a)-(b) depict an experimental setup of the tumor motion compensation system;

FIG. 23 depicts table motion in X and Y direction for the tumor tracking test;

FIG. 24 depicts comparison of the stationary plan with the tracking one, where FIG. 24(a) depicts inplane profile, FIG. 24(b) depicts crossplane profile, FIG. 24(c) depicts passing criteria is critical in the high gradient region and FIG. 24(d) depicts 3D dose profile for both plans;

FIG. 25 depicts comparison of the IMRT plan with the tracking one, where FIG. 25(a) depicts inplane profile, FIG. 25(b) depicts crossplane profile, and FIG. 25(c) depicts diagonal profile;

FIG. 26(a) depicts imaging lung tumors while the supporting couch is stationary; FIG. 26(b) depicts imaging the patient's posterior left lung tumor while the supporting couch is programmed to undergo counter-motion to this tumor's physiological motion; FIG. 26(c) depicts imaging the patient's anterior right lung tumor while the supporting couch is programmed to undergo counter-motion to this tumor's physiological motion; In FIG. 26(d), by segmenting and combining the non motion-blurred parts of FIGS. 26(a)-(c), a fully motion-compensated image set can be obtained;

FIG. 27 depicts an apparatus according to one embodiment of the present invention;

FIG. 28 depicts a tracking strategy, i.e., level of tracking of the tumor;

FIG. 29(a) depicts tumor tracking error for motorized platform when PID controller was used with different subject load; FIG. 29(b) depicts tumor tracking error for motorized platform when adaptive controller was uses with different subject loads;

FIG. 30 depicts sessions with 1 channel of skin conductance;

FIG. 31 depicts multi-modality sessions with BVP (amplitude) and Temp;

FIG. 32 depicts line graphs of the raw BVP or EKG signal and of the abdominal and thoracic respiration;

FIG. 33 depicts trend graphs of the total and percent power for the three standard HRV frequency bands, VLF, LF and HF;

FIG. 34 depicts a schematic diagram of the proposed methodology;

FIG. 35 depicts a physiological sensor suite and data acquisition equipment, where FIG. 35(a) depicts an EMG Sensor, FIG. 35(b) depicts an EKG Sensor, FIG. 35(c) depicts a BVP Sensor, FIG. 35(d) depicts a Temp. Sensor, FIG. 35(e) depicts a Skin Conductance Sensor, FIG. 35(f) depicts a Respiration Sensor, and FIG. 35(g) depicts a Flexcomp Infiniti (data acquisition module);

FIG. 36 depicts motion capturing systems (Aurora EM sensors); FIG. 36(a) depicts Aurora EM Sensor package, and FIG. 36(b) depicts Aurora EM sensor (0.9 mm×6 mm);

FIG. 37 depicts a CyberKnife robotic system for radiation treatment;

FIG. 38 depicts normal and irregular respiration signals (representative cases);

FIG. 39 depicts a range of the tumor motion within 2 cm for normal respiration in each direction, and where the respiration cycle was 3.5-7.3 s;

FIG. 40 depicts a tracking error limit of 3 mm; and

FIG. 41 depicts dependency of residual errors to the lung doses.

### DETAILED DESCRIPTION

It should be understood that this invention is not limited to the particular methodology, protocols, etc., described herein and as such may vary. The terminology used herein is for the purpose of describing particular embodiments only, and is not intended to limit the scope of the present invention, which is defined solely by the claims.

As used herein and in the claims, the singular forms include the plural reference and vice versa unless the context clearly indicates otherwise. Other than in the operating examples, or where otherwise indicated, all numbers expressing quantities used herein should be understood as modified in all instances by the term “about.”

All publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

Some Selected Definitions

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments of the aspects described herein, and are not intended to limit the claimed invention, because the scope of the invention is limited only by the claims. Further, unless otherwise required by context, singular terms shall include pluralities and plural terms shall include the singular.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Other than in the operating examples, or where otherwise indicated, all numbers expressing quantities used herein should be understood as modified in all instances by the term “about.” The term “about” when used in connection with percentages may mean±1%.

The singular terms “a,” “an,” and “the” include plural referents unless context clearly indicates otherwise. Similarly, the word “or” is intended to include “and” unless the context clearly indicates otherwise. Thus for example, references to “the method” includes one or more methods, and/or steps of the type described herein and/or which will become apparent to those persons skilled in the art upon reading this disclosure and so forth.

Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments of the aspects described herein, the subject is a mammal, e.g., a primate, e.g., a human. The terms, “patient” and “subject” are used interchangeably herein.

In some embodiments, the subject is a mammal. The mammal can be a human, non-human primate, mouse, rat, dog, cat, horse, or cow, but are not limited to these examples. Mammals other than humans can be advantageously used as subjects that represent animal models of disorders.

A subject can be one who has been previously diagnosed with or identified as suffering from or having a disease or disorder caused by any microbes or pathogens described herein. By way of example only, a subject can be diagnosed with sepsis, inflammatory diseases, or infections.

To the extent not already indicated, it will be understood by those of ordinary skill in the art that any one of the various embodiments herein described and illustrated may be further modified to incorporate features shown in any of the other embodiments disclosed herein.

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### Part 1

In one embodiment, the present invention is directed to clinical implementation of real-time tumor motion tracking and 4D adaptive radiotherapy using a multi-leaf collimator (MLC), and/or an MLC-carriage, and/or a treatment table or couch. The present invention includes disclosure relating to use of the MLC, MLC-carriage and/or couch; tumor motion decomposition and allocation to subsystems (MLC, MLC-carriage, couch); tumor motion assessment, patient's condition assessment for tracking tolerance; correlation and prediction of internal tumor motion and external surrogate motion; level of tracking determination (whether soft, moderate or extreme tracking), which is a key feature of the present invention that makes the present technology sufficiently patient-friendly, user-friendly and clinically robust for realistic implementation; PTV margin determination; multiple or adaptive planning; real-time tumor tracking and dynamic delivery of radiation; re-evaluation of tumor response and patient's condition; and randomized trials. It is noted that at CT-simulation stage and/or initial radiation therapy delivery (or subsequent periodic verification) stage, the planned couch tracking motion can be carried out, if the tracking couch of the present invention has been installed in said CT-simulation and/or radiation delivery suites. Upon such tracking motion, the moving tumor shall appear under imaging to be either stationary, moving marginally, or moving with reduced excursion, depending on the tracking strategy chosen (aggressive, moderate, or soft). This is an added confirmation of the tracking strategy being deployed for the patient.

The present invention overcomes a known problem whereby irregular motion of a tumor may marginalize efficacy. A suitable prediction methodology may be used to overcome problems associated with irregular motion of the tumor.

This invention disclosure teaches a method, implementation technique and workflow for realistic clinical utilization of tumor tracking strategies involving the selection of aggressive, moderate or soft couch tracking motions as prescribed by the clinician and/or preferred by the patient.

Having different levels of tracking available to choose between, and an associated method of selecting which of these tracking levels is most suitable for each patient or treatment.

### Part 2

### Specification

Undesired motions of the tumors in the thoracic and abdominal regions can be tracked and compensated deploying multi-leaf collimator (MLC), MLC-carriage (MLC-bank) and/or patient positioning couch (table) for precise delivery of radiation dose to the target sparing adjacent healthy tissues and critical structures. However, clinical implementations of these technologies require strategies that would be executed methodically for safe and reliable usage of the said technologies in the clinic. The strategies are depicted as follows:

a. Clinical CT-Sim Stage
i. Acquire real-time trajectory of tumor using sensory systems (electromagnetic (EM), ultrasound (U/S), infrared, CT/4D-CT, 4D-Conebeam CT (4D-CBCT), etc.). May use internal fiducial and/or external surrogates (fiducial or marks or LED, etc.).
ii. Assess tumor motion amplitude, velocity, nature and adjacent tissues; patient's health condition, breathing pattern, etc. Determine the tumor motion and correlate to external surrogate markers.
iii. Determine whether the tumor tracking is required. If required, assess whether the patient would be able to tolerate motion induced by tracking (if any, e.g., tracking using couch). Determine level of tracking: (1) very accurate tracking, (2) not so accurate, i.e., tracking the gross motion of the tumor, neglecting sharp changes or high frequency components.
iv. Determine whether decomposition of 3D tumor motion is required; if so, determine suitable allocation of tumor motion to the linac subsystems (MLC, MLC-carriage and couch).
v. Determine margins for the planning target volume (PTV) considering patient's condition and the level of tracking (soft, moderate or exact/extreme).

b. Dosimetric Planning Stage
i. Generate at least two plans: (1) plan that could be used without tumor tracking, (2) plan that would be used with tracking, i.e., tighter margin to clinical target volume (CTV) for generating PTV.

c. Radiation Treatment Delivery
i. Access the patient condition, acquire the tumor motion. Compare the current tumor motion with that acquired during CT-sim. Verify or re-establish the tumor motion to the motion of the external surrogate marker. Have clinical judgment for tracking.
ii. Based on the clinical assessment deliver treatment with or without tracking. Determine the level of tracking whether soft, moderate or exact/extreme tracking is applicable/appropriate.

d. Patient Follow-Up Stage
i. Follow-up the patient at least once a week to access the tumor response and patient's condition (especially that associated to tumor tracking, i.e. tumor coverage, adequacy/shrinkage of CTV-PTV margin, dosimetric re-planning or adaptive planning, patient's tolerance, etc). Use clinically appropriate assessment modality such as CT, PET, MR, U/S imaging if necessary.
ii. Long term follow-up is desirable. Multi institute randomize trials of tracking vs. no-tracking treatment study may be pursued.

In one embodiment, the steps may progress in the order provided above, i.e., step a.i, then step a.ii, a.iii, a.iv, a.v, b.i, c.i, c.ii, d.i and d.ii. However, any suitable order of steps may be utilized.

The present invention includes tracking tumors in thoracic and abdominal regions for radiation therapy using: (a) MLC and/or MLC-bank (i.e., MLC-carriage), (b) Couch (Table), (c) combination of MLC/MLC-bank and Couch.

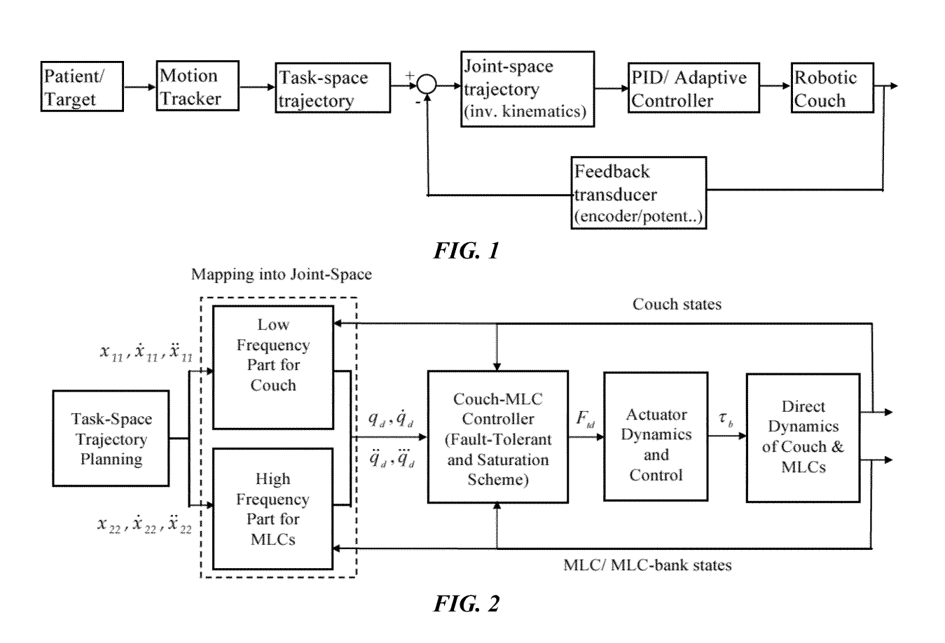

The concept is depicted in FIGS. 1-2. The real-time tumor trajectories are to be decomposed and allocated to appropriate subsystems (MLC, MLC-bank, and couch) based on the tumor motion characteristics and condition of the patient. The said decomposition of the tumor trajectories can be in simple orthogonal directions (patient's superior-inferior, medial-lateral and anterior-posterior directions when the patient is laid on the treatment table) to the radiation beam or complex frequency-wise (high- and low-frequency). Additionally, two novel algorithms (acceleration-enhanced (AE) ANN and nLMS) for predicting tumor motion have been developed for using in tumor tracking during radiation therapy.

FIG. 1 depicts closed-loop control of a robotic couch for compensating respiratory motion of a tumor.

FIG. 2 depicts a preliminary schematic of a decentralized coordinated dynamics-based closed-loop controller for a couch and an MLC (MLC-bank/carriage).

### Part 3

### Novel Acceleration-Enhanced Method for Prediction of Motion in Normal and Irregular Respiration for Tumor Motion Compensation

The prediction of the respiration motion that induces tumor motion is one of the most important steps in active tracking of tumor and dynamic delivery of radiation dose to tumor. We have developed a novel acceleration-enhanced (AE) method with predicted acceleration and ratio between the real and predicted acceleration taken into account. The AE method can be applied to traditional adaptive filters. We have compared the performances of normalized least mean squares (nLMS), artificial neural network (ANN), and their AE counterparts for predicting the respiration motion during normal and irregular respiration. The results revealed that the AE filters outperformed their traditional counterparts. The AE-ANN filter had the best performance in the prediction of normal respiration motion, whereas the AE-nLMS filter excelled in the prediction of irregular respiration motion.

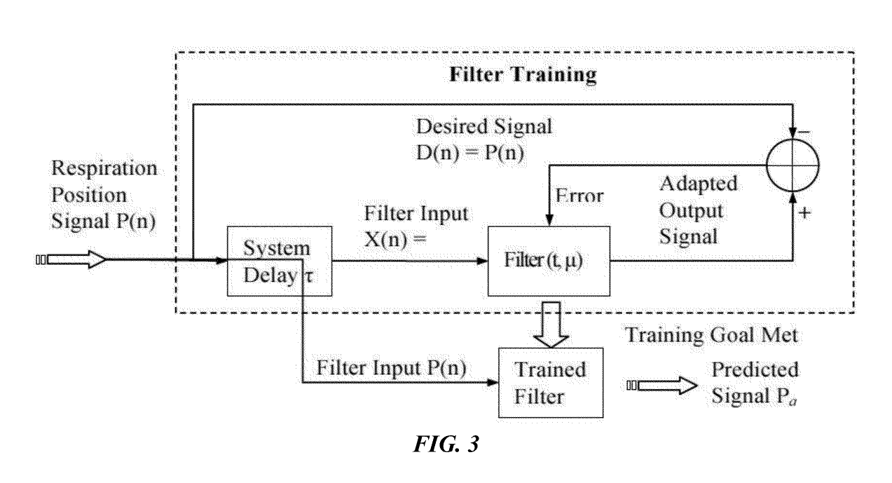

FIG. 3 is a schematic of Adaptive Filter Training.

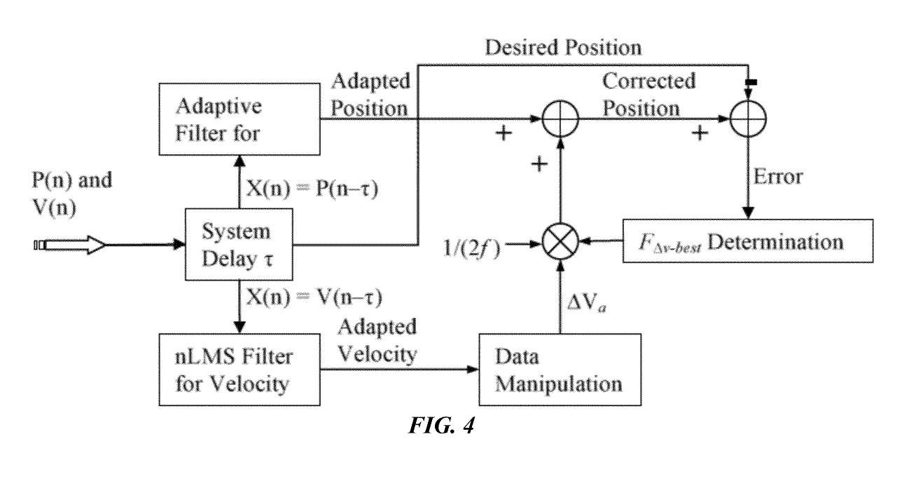

FIG. 4 is a schematic of the Factor Determination Process for AE Filters.

The adaptive ANN, nLMS, AE-ANN, and AE-nLMS filters were tested with normal respiration signal with system latency of 125 ms, 187.5 ms, and 250 ms, as well as irregular respiration signal with latency of 156.3 ms and 250 ms.

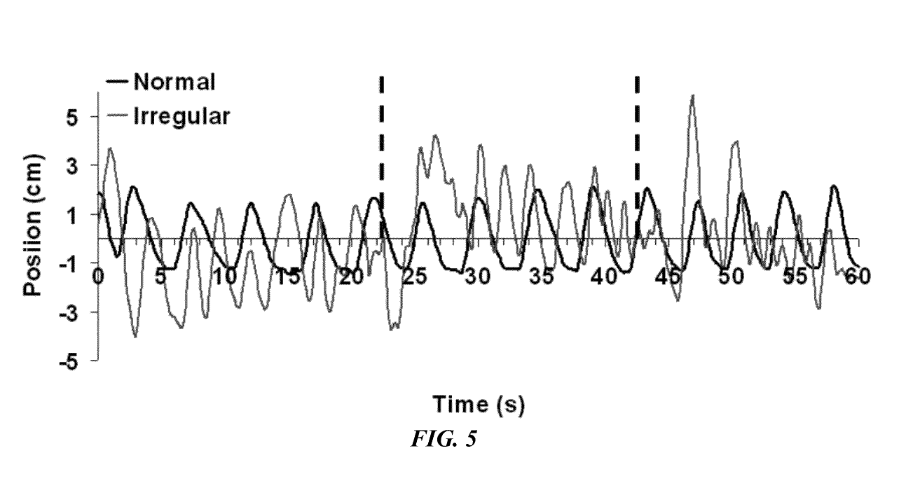

FIG. 5 is a chart of Normal and Irregular Respiration Signals. In FIG. 5, Time (s) is provided along the x-axis and Position (cm) is provided along the y-axis.

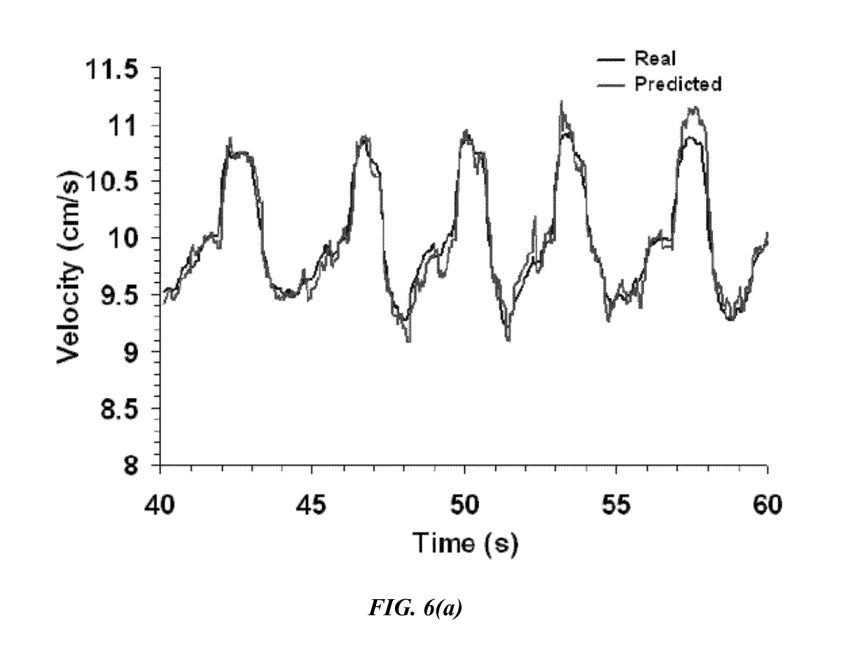

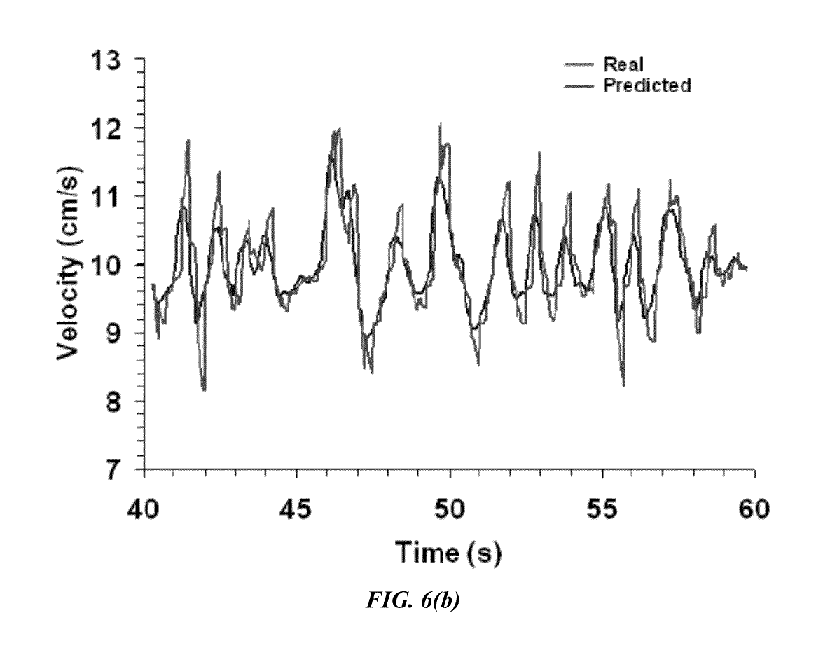

FIGS. 6(a)-(b) are charts of velocities predicted by the adaptive nLMS filter in the acceleration-enhanced method for (FIG. 6(a)) normal respiration at 125 ms, and for (FIG. 6(b)) irregular respiration at 250 ms. In FIG. 6, Time (s) is provided along the x-axis and Velocity (cm/s) is provided along the y-axis.

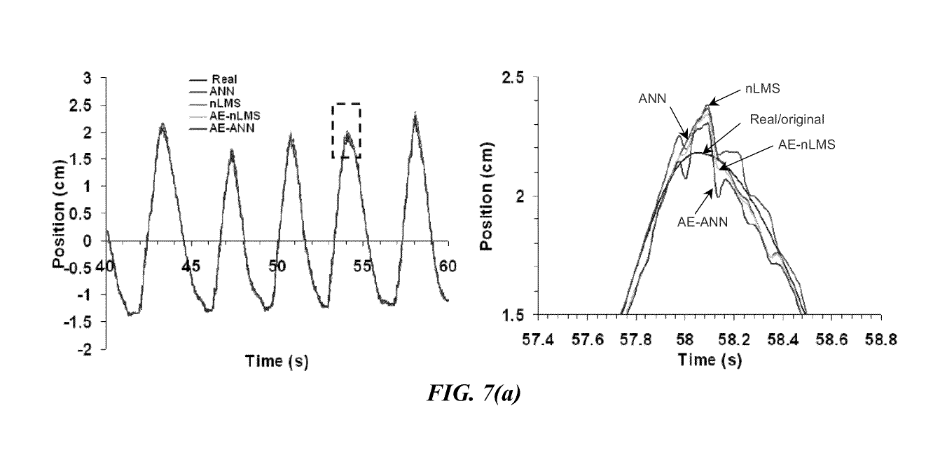

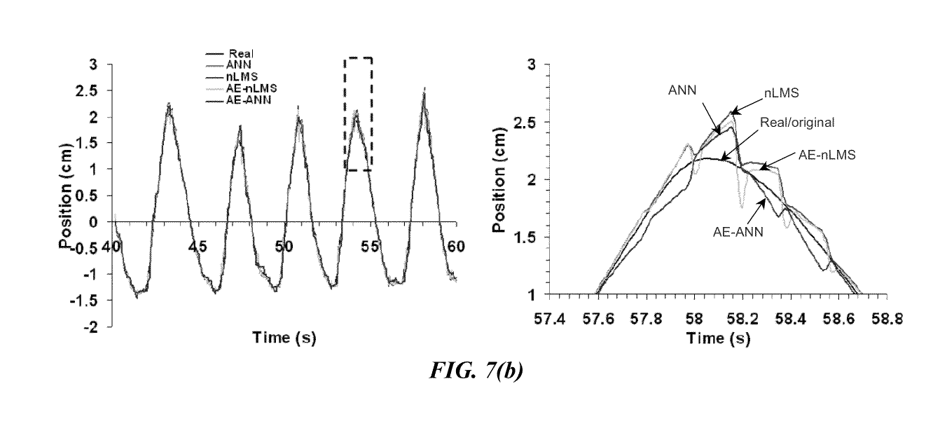

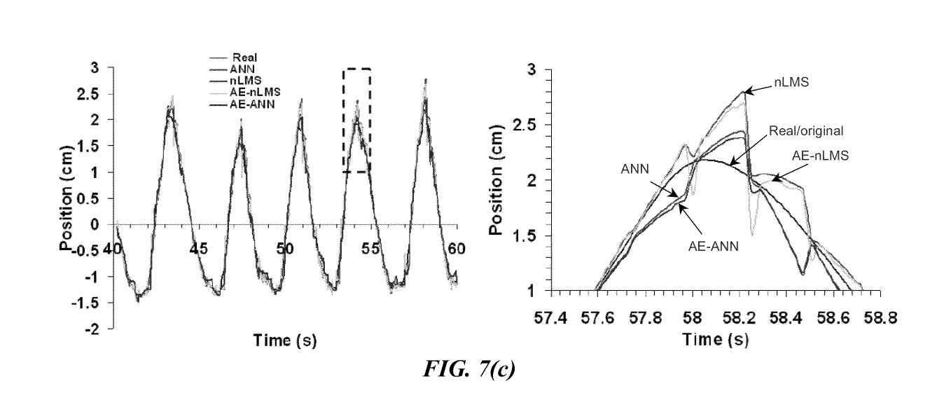

FIGS. 7(a)-(c) are charts of positions predicted by the adaptive ANN, nLMS, AE-nLMS, and AE-ANN filters in normal respiration, and the close-ups of the dotted boxes in the left panels at the times of (FIG. 7(a)) 125 ms, (FIG. 7(b)) 187.5 ms, and (FIG. 7(c)) 250 ms. In FIG. 7, Time (s) is provided along the x-axis and Position (cm) is provided along the y-axis.

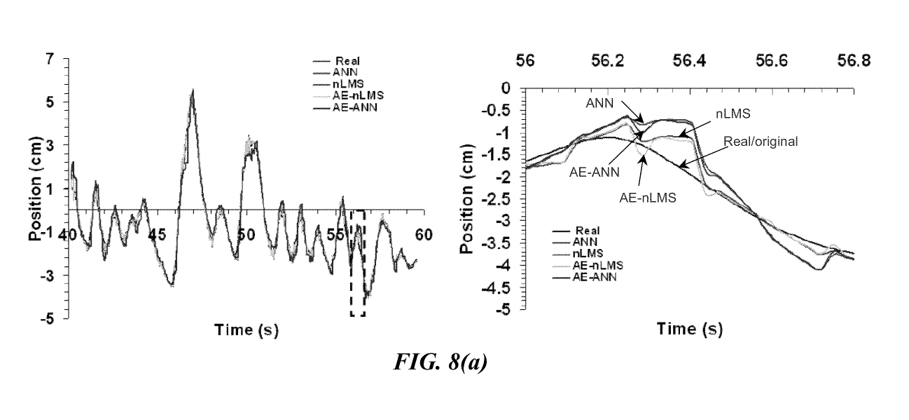

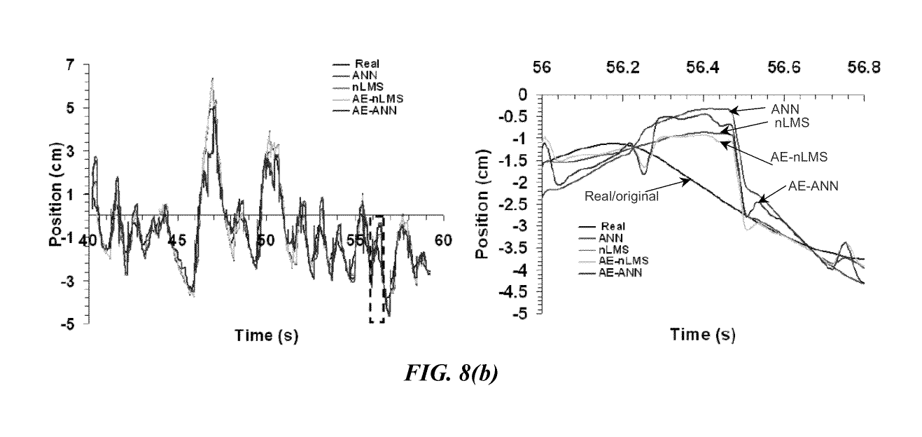

FIGS. 8(a)-(b) are charts of positions predicted by the adaptive ANN, nLMS, AE-nLMS, and AE-ANN filters from the real position in the irregular respiration, as well as the close-ups of the dotted boxes at the times of (FIG. 8(a)) 156 ms, and (FIG. 8(b)) 250 ms. In FIG. 8, Time (s) is provided along the x-axis and Position (cm) is provided along the y-axis.

In this study, it is indicated that in normal respiration, the adaptive ANN and AE-ANN filters provide better accuracy in prediction than the adaptive nLMS and AE-nLMS filters. Whereas in the case of irregular respiration, the predictions given by the adaptive nLMS and AE-nLMS filters are more accurate than those given by the adaptive ANN and AE-ANN filters.

Prediction with both position and velocity in filter improves the accuracy of the prediction by having the acceleration taken into account. The acceleration-enhanced method is able to improve the performance of the ANN and nLMS filters. The adaptive AE-ANN filter gives the best accuracy in the prediction for normal respiration, whereas the adaptive AE-nLMS filter gives the minimum error in the prediction for irregular respiration. This method can also be implemented to other filters.

### Part 4

### Dosimetric Effects Due to Prediction Errors or Residual Motion (as if the Tumor Moved Permanently by “x” Amount for the Whole Duration of Treatment)

The change of dose induced by the residual in prediction has been studied. The subsequence of the residual in prediction is that the iso-center of the beams is shifted from where it should be by an amount which is equal to the residual. The changes on dose delivered to Planning Target Volume (PTV), Clinical target volume (CTV), Lung, Spinal Cord, and Carina of five patients are measured by shifting the iso-center of the beams away from its original place and these results are included in Table 1 has the data from this study. The shifts of iso-center along all X, Y, and Z directions (these three directions are the same as patient's medial-lateral superior-inferior, and anterior-posterior directions) by 2 mm, 3.5 mm, and 5 mm are measured, with both the average and maximum changes on delivered doses calculated in Gray and in percentage.

TABLE 1
Dosimetric data from the patients' plan (Initial => original clinical
plan; 2 mm, 3.5 mm, and 5 mm mean that the tumor, i.e., the plan iso-center has been moved that
distances to simulate the residual motion/error).

| | PTV dose | | | | | | GTV dose | | | | | |
|---|---|---|---|---|---|---|---|---|---|---|---|---|
| | | | | | Max | Mean | | | | | Max | Mean |
| Patient 1 | D99 | D95 | D50 | D5 | dose | dose | D99 | D95 | D50 | D5 | dose | dose |
| Phase | (Gy) | (Gy) | (Gy) | (Gy) | (Gy) | (Gy) | (Gy) | (Gy) | (Gy) | (Gy) | (Gy) | (Gy) |
| Initial | 49.04 | 50.3 | 53.08 | 54.87 | 55.56 | 52.91 | 52.01 | 52.77 | 54.08 | 55.16 | 55.56 | 54.04 |
| 2 mm | 49.18 | 50.69 | 53.14 | 55.17 | 55.87 | 53.18 | 52.16 | 52.86 | 54.28 | 55.41 | 55.87 | 54.23 |
| 3.5 mm | 48.66 | 50.56 | 53.53 | 55.57 | 56.26 | 53.35 | 52.02 | 52.79 | 54.49 | 55.82 | 56.19 | 54.43 |
| 5 mm | 47.52 | 49.96 | 53.54 | 55.82 | 56.7 | 53.3 | 51.58 | 52.53 | 54.54 | 56.05 | 56.51 | 54.46 |
| | Lung Vx [%] | | | | | Spinal Cord | | | Carina | | | |
| | | | | | Mean | Max | Mean | | Max | Mean | | |
| | | | | | dose | dose | dose | D5 | dose | dose | D5 | |
| Phase | V5 | V13 | V20 | V30 | (Gy) | (Gy) | (Gy) | (Gy) | (Gy) | (Gy) | (Gy) | |
| Initial | 17.08 | 10.28 | 8.56 | 6.29 | 5.07 | 6.37 | 2.5 | 6.12 | 1.69 | 1.39 | 1.58 | |
| 2 mm | 18.33 | 11 | 9.23 | 6.88 | 5.43 | 6.38 | 2.54 | 6.15 | 1.83 | 1.45 | 1.68 | |
| 3.5 mm | 19.05 | 11.58 | 9.73 | 7.32 | 5.71 | 6.39 | 2.57 | 6.16 | 1.99 | 1.23 | 1.77 | |
| 5 mm | 19.93 | 12.12 | 10.26 | 7.74 | 5.98 | 6.42 | 2.6 | 6.18 | 2.14 | 1.56 | 1.89 | |
| | PTV dose | | | | | | GTV dose | | | | | |
| | | | | | Max | Mean | | | | | Max | Mean |
| Patient 2 | D99 | D95 | D50 | D5 | dose | dose | D99 | D95 | D50 | D5 | dose | dose |
| Phase | (Gy) | (Gy) | (Gy) | (Gy) | (Gy) | (Gy) | (Gy) | (Gy) | (Gy) | (Gy) | (Gy) | (Gy) |
| Initial | 48.56 | 49.82 | 51.94 | 52.99 | 53.76 | 51.74 | 51.47 | 51.68 | 52.38 | 52.92 | 53.47 | 52.34 |
| 2 mm | 47.39 | 49.6 | 51.6 | 52.82 | 53.69 | 51.5 | 51.32 | 51.54 | 52.15 | 52.68 | 53.1 | 52.13 |
| 3.5 mm | 45.75 | 48.86 | 51.36 | 52.58 | 53.42 | 51.11 | 50.92 | 51.18 | 51.82 | 52.48 | 53.02 | 51.82 |
| 5 mm | 43.74 | 47.66 | 51.36 | 52.61 | 53.42 | 50.93 | 50.57 | 51.15 | 51.72 | 52.42 | 52.96 | 51.74 |
| | Lung Vx [%] | | | | | Spinal Cord | | | | | | |
| | | | | | Mean | Max | Mean | | | | | |
| | | | | | dose | dose | dose | D5 | | | | |
| Phase | V5 | V13 | V20 | V30 | (Gy) | (Gy) | (Gy) | (Gy) | | | | |
| Initial | 20.9 | 17.24 | 12.72 | 8.32 | 6.18 | 31.93 | 3.63 | 23.57 | | | | |
| 2 mm | 20.88 | 17.35 | 13.05 | 8.76 | 6.35 | 32.16 | 4.07 | 27.14 | | | | |
| 3.5 mm | 20.81 | 17.34 | 13.26 | 8.97 | 6.44 | 31.82 | 4.34 | 28.8 | | | | |
| 5 mm | 20.74 | 17.39 | 13.56 | 9.22 | 6.57 | 31.87 | 4.62 | 29.81 | | | | |
| | PTV dose | | | | | | GTV dose | | | | | |
| | | | | | Max | Mean | | | | | Max | Mean |
| Patient 3 | D99 | D95 | D50 | D5 | dose | dose | D99 | D95 | D50 | D5 | dose | dose |
| Phase | (Gy) | (Gy) | (Gy) | (Gy) | (Gy) | (Gy) | (Gy) | (Gy) | (Gy) | (Gy) | (Gy) | (Gy) |
| Initial | 48.72 | 49.88 | 52.56 | 53.47 | 53.76 | 52.21 | 52.7 | 52.88 | 53.28 | 53.63 | 53.75 | 53.28 |
| 2 mm | 46.61 | 48.74 | 52.41 | 53.54 | 54 | 52.03 | 52.51 | 52.84 | 53.33 | 53.65 | 53.78 | 53.3 |
| 3.5 mm | 42.58 | 46.56 | 52.46 | 53.45 | 54.13 | 51.51 | 51.69 | 52.36 | 53.19 | 53.56 | 53.74 | 53.11 |
| 5 mm | 37.28 | 43.3 | 52.36 | 53.44 | 54.12 | 50.88 | 50.59 | 51.7 | 53.15 | 53.52 | 53.63 | 52.97 |
| | Lung Vx [%] | | | | | Spinal Cord | | | Carina | | | |
| | | | | | Mean | Max | Mean | | Max | Mean | | |
| | | | | | dose | dose | dose | D5 | dose | dose | D5 | |
| Phase | V5 | V13 | V20 | V30 | (Gy) | (Gy) | (Gy) | (Gy) | (Gy) | (Gy) | (Gy) | |
| Initial | 30 | 13.01 | 7.74 | 3.68 | 6.06 | 14.28 | 2.37 | 13.32 | 20.96 | 9.74 | 16.33 | |
| 2 mm | 29.98 | 12.83 | 7.34 | 3.45 | 5.98 | 15.54 | 2.44 | 13.58 | 21.38 | 9.29 | 17.32 | |
| 3.5 mm | 30.01 | 12.63 | 6.97 | 3.23 | 5.91 | 14.69 | 2.48 | 13.74 | 21.34 | 8.97 | 17.39 | |
| 5 mm | 29.73 | 12.41 | 6.62 | 3.04 | 5.84 | 14.9 | 2.51 | 13.9 | 21.16 | 8.69 | 16.73 | |
| | PTV dose | | | | | | GTV dose | | | | | |
| | | | | | Max | Mean | | | | | Max | Mean |
| Patient 4 | D99 | D95 | D50 | D5 | dose | dose | D99 | D95 | D50 | D5 | dose | dose |
| Phase | (Gy) | (Gy) | (Gy) | (Gy) | (Gy) | (Gy) | (Gy) | (Gy) | (Gy) | (Gy) | (Gy) | (Gy) |
| Initial | 49.59 | 50.03 | 51.17 | 53.32 | 54.94 | 51.34 | 50.45 | 50.68 | 51.74 | 53.67 | 54.94 | 51.89 |
| 2 mm | 49.77 | 50.45 | 52.51 | 54.57 | 56.24 | 52.5 | 50.73 | 51.24 | 53.05 | 54.89 | 56.24 | 53.05 |
| 3.5 mm | 49.14 | 50.08 | 53.12 | 55.25 | 56.84 | 52.94 | 50.36 | 51.07 | 53.68 | 55.56 | 56.84 | 53.52 |
| 5 mm | 47.64 | 48.93 | 52.96 | 55.35 | 56.9 | 52.68 | 49.12 | 50.14 | 53.3 | 55.62 | 56.9 | 53.3 |
| | Lung Vx [%] | | | | | Spinal Cord | | | | | | |
| | | | | | Mean | Max | Mean | | | | | |
| | | | | | dose | dose | dose | D5 | | | | |
| Phase | V5 | V13 | V20 | V30 | (Gy) | (Gy) | (Gy) | (Gy) | | | | |
| Initial | 24.01 | 13.52 | 8.53 | 4.89 | 5.23 | 2.58 | 0.28 | 1.15 | | | | |
| 2 mm | 24.09 | 13.35 | 8.53 | 4.79 | 5.24 | 4.46 | 0.34 | 1.58 | | | | |
| 3.5 mm | 23.94 | 13.22 | 8.56 | 4.67 | 5.22 | 5.92 | 0.44 | 2.35 | | | | |
| 5 mm | 23.74 | 13 | 8.46 | 4.48 | 5.15 | 6.4 | 0.57 | 3.5 | | | | |
| | PTV dose | | | | | | GTV dose | | | | | |
| | | | | | Max | Mean | | | | | Max | Mean |
| Patient 5 | D99 | D95 | D50 | D5 | dose | dose | D99 | D95 | D50 | D5 | dose | dose |
| Phase | (Gy) | (Gy) | (Gy) | (Gy) | (Gy) | (Gy) | (Gy) | (Gy) | (Gy) | (Gy) | (Gy) | (Gy) |
| Initial | 48.65 | 50.24 | 52.57 | 53.29 | 53.69 | 52.28 | 52.13 | 52.29 | 52.77 | 53.32 | 53.59 | 52.78 |
| 2 mm | 47.8 | 49.84 | 52.64 | 53.4 | 53.83 | 52.26 | 52.12 | 52.37 | 52.82 | 53.33 | 53.66 | 52.83 |
| 3.5 mm | 45.97 | 49.18 | 52.55 | 53.38 | 53.93 | 52.06 | 51.89 | 52.24 | 52.73 | 53.26 | 53.61 | 52.74 |
| 5 mm | 43.44 | 48.12 | 52.58 | 53.42 | 54.06 | 51.91 | 51.54 | 52.14 | 52.74 | 53.26 | 53.75 | 52.73 |
| | Lung Vx [%] | | | | | Spinal Cord | | | Carina | | | |
| | | | | | Mean | Max | Mean | | Max | Mean | | |
| | | | | | dose | dose | dose | D5 | dose | dose | D5 | |
| Phase | V5 | V13 | V20 | V30 | (Gy) | (Gy) | (Gy) | (Gy) | (Gy) | (Gy) | (Gy) | |
| Initial | 34.53 | 15.67 | 13.57 | 11.37 | 7.66 | 6.82 | 0.82 | 2.56 | 17.25 | 9.12 | 11.6 | |
| 2 mm | 34.93 | 15.71 | 13.68 | 11.37 | 7.69 | 8.39 | 0.97 | 3.5 | 18.36 | 9.52 | 11.9 | |
| 3.5 mm | 35.25 | 15.77 | 13.73 | 11.34 | 7.7 | 9.37 | 1.01 | 3.56 | 19.5 | 9.77 | 12.13 | |
| 5 mm | 35.62 | 15.77 | 13.68 | 11.31 | 7.71 | 10.61 | 1.07 | 3.63 | 21.37 | 10.02 | 12.36 | |

TABLE 2
Effects of residual motions (or errors) on dose distribution.

| | D99 | | | | D95 | | | | D50 | | | |
|---|---|---|---|---|---|---|---|---|---|---|---|---|
| | | with | | | | with | | | | with | | |
| | planned | prediction | difference | | planned | prediction | difference | | planned | prediction | Difference | |
| Pt. | (Gy) | | (Gy) | (%) | (Gy) | | (Gy) | (%) | (Gy) | | (Gy) | (%) |
| | PTV | | | | | | | | | | | |
| 1 | 49.04 | 48.92 | −0.12 | −0.25 | 50.3 | 50.24 | −0.06 | −0.11 | 53.08 | 53.06 | −0.02 | −0.03 |
| 2 | 49.57 | 49.52 | −0.05 | −0.11 | 50.9 | 50.84 | −0.06 | −0.12 | 52.37 | 52.36 | −0.01 | −0.01 |
| 3 | 48.5 | 45.55 | −2.95 | −6.07 | 50.67 | 49.52 | −1.15 | −2.28 | 54.31 | 54.27 | −0.05 | −0.08 |
| 4 | 49.41 | 49.25 | −0.16 | −0.32 | 50.74 | 50.65 | −0.1 | −0.19 | 52.77 | 52.72 | −0.05 | −0.1 |
| 5 | 61.95 | 61.89 | −0.06 | −0.1 | 62.49 | 62.51 | 0.02 | 0.03 | 64.83 | 64.78 | −0.05 | −0.08 |
| average | | | −0.67 | −1.37 | | | −0.27 | −0.53 | | | −0.03 | −0.06 |
| | CTV | | | | | | | | | | | |
| 1 | 52.01 | 51.98 | −0.03 | −0.05 | 52.77 | 52.74 | −0.03 | −0.06 | 54.08 | 54.06 | −0.02 | −0.05 |
| 2 | 51.6 | 51.62 | 0.02 | 0.04 | 51.81 | 51.8 | −0.01 | −0.01 | 52.37 | 52.39 | 0.02 | 0.03 |
| 3 | 53.54 | 53.05 | −0.49 | −0.91 | 54.1 | 53.92 | −0.18 | −0.33 | 54.66 | 54.62 | −0.04 | −0.08 |
| 4 | 52.47 | 52.39 | −0.08 | −0.14 | 52.57 | 52.5 | −0.07 | −0.14 | 53.14 | 53.08 | −0.07 | −0.12 |
| 5 | 63.62 | 63.63 | 0.01 | 0.02 | 63.89 | 63.89 | 0 | 0 | 65.1 | 65.13 | 0.03 | 0.04 |

| | | | | | | |
|---|---|---|---|---|---|---|
| average | −0.11 | −0.21 | −0.06 | −0.11 | −0.02 | −0.03 |

| | Lung | | | Spinal Coord | | | Carina | | |
|---|---|---|---|---|---|---|---|---|---|
| | V20 | | | D5 | | | D5 | | |
| | | with | | | with | | | with | |
| | planned | prediction | diff. | planned | prediction | diff. | planned | prediction | diff. |
| Pt. | (%) | | (%) | (Gy) | | (Gy) | (Gy) | | (Gy) |
| 1 | 8.56 | 8.52 | −0.04 | 6.12 | 6.12 | 0 | 1.58 | 1.58 | 0 |
| 2 | 2.25 | 2.25 | 0 | 8.28 | 8.28 | 0 | | | |
| 3 | 15.4 | 15.26 | −0.15 | 4.33 | 4.33 | 0 | 7.34 | 7.59 | 0.25 |
| 4 | 8.79 | 8.76 | −0.03 | 16.61 | 16.61 | 0 | 19.83 | 19.99 | 0.16 |
| 5 | 11.18 | 11.19 | 0.01 | 4.81 | 4.81 | 0 | | | |

| | | | |
|---|---|---|---|
| average | −0.04 | 0 | 0.14 |

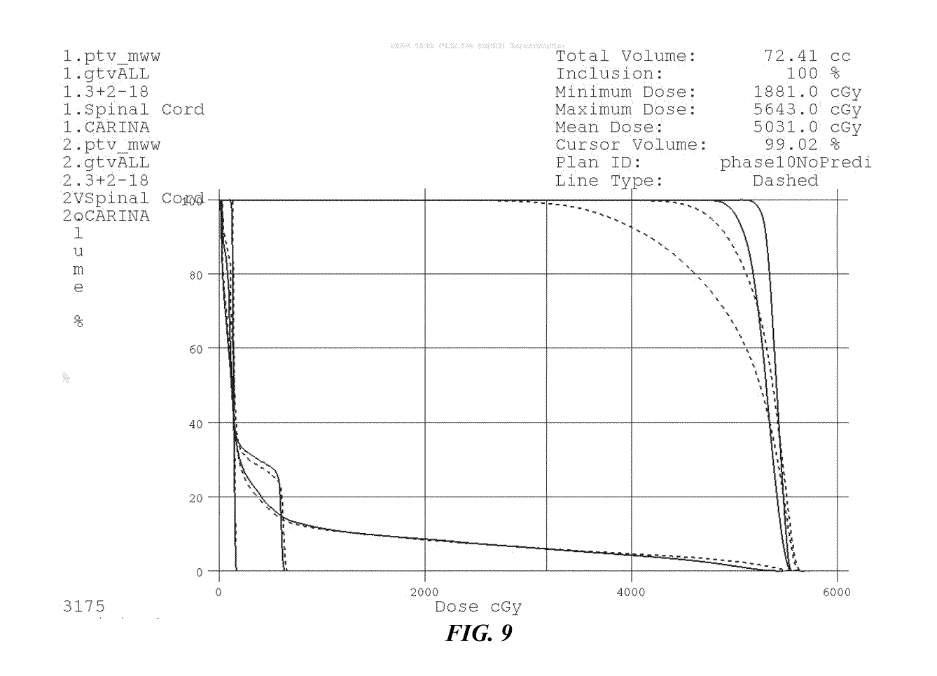

FIG. 9 is a chart of DVH for phase 10 without tumor motion compensation or prediction (one of the representative patient case). Movements of the tumor on X, Y, Z directions are −11.1 mm, 4.2 mm, and −13.9 mm, respectively. In FIG. 9, Dose (cGy) is provided along the x-axis and Volume (%) is provided along the y-axis.

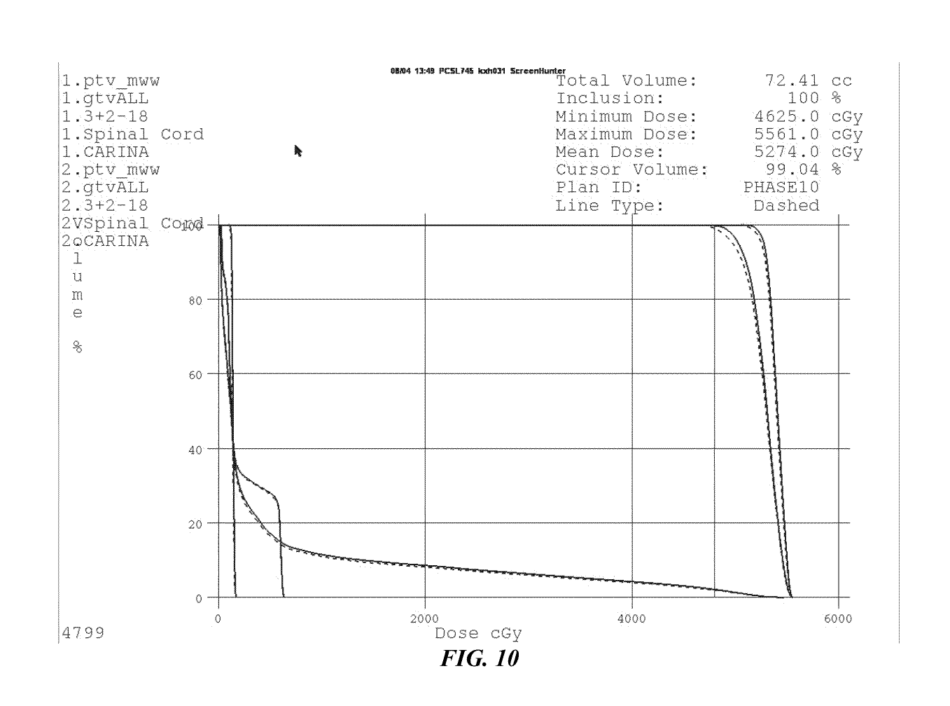

FIG. 10 is a chart of DVH with tumor motion compensation (one of the representative patient case). The differences the dose distributions are due to predict error (or residual motion). In FIG. 10, Dose (cGy) is provided along the x-axis and Volume (%) is provided along the y-axis.

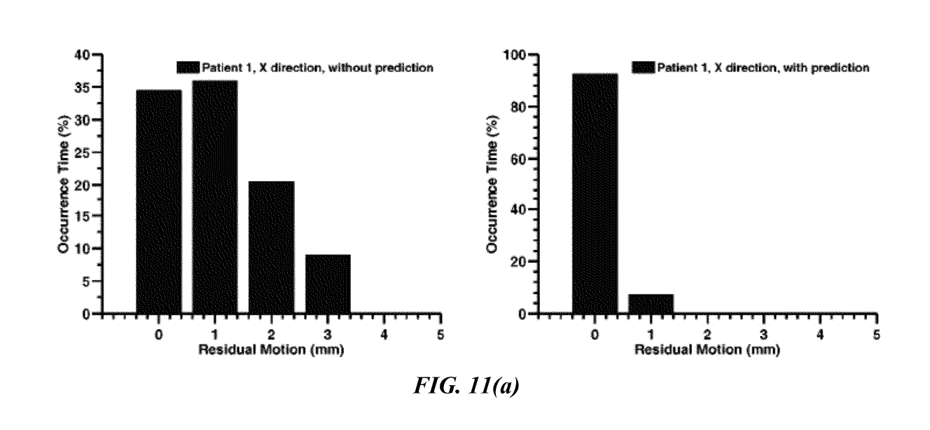

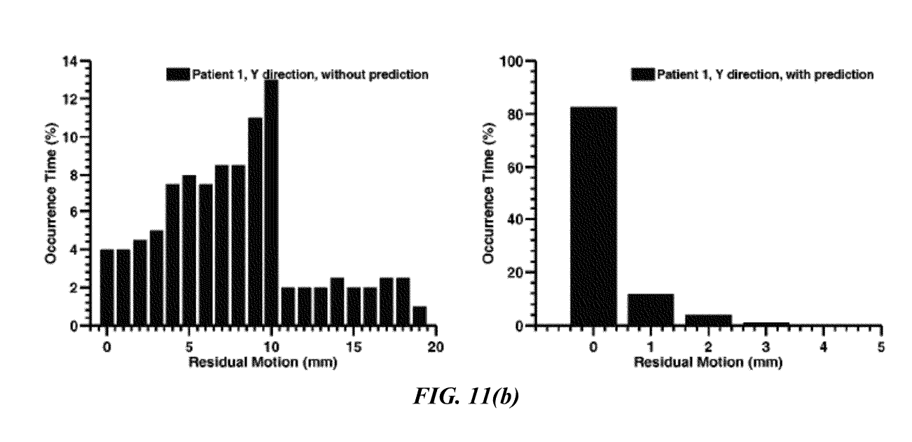

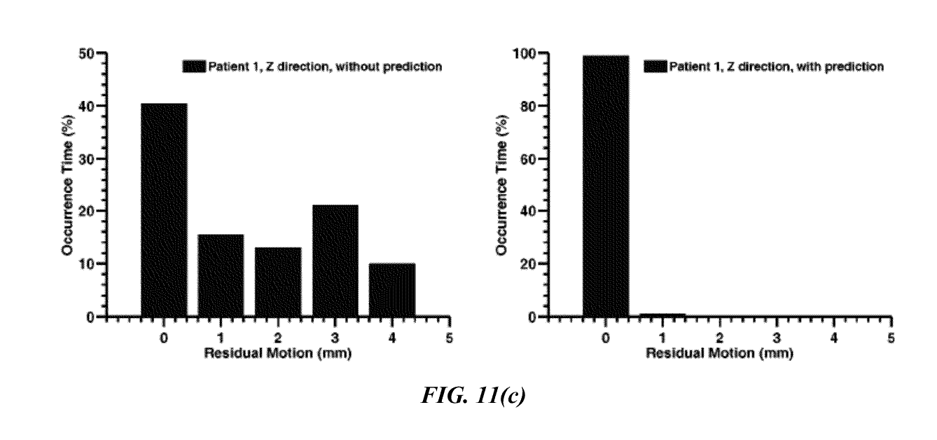

FIGS. 11(a)-(c) are histograms of the residual motion of the tumor with and without tracking (prediction) on (FIG. 11(a)) X direction, (FIG. 11(b)) Y direction, and (FIG. 11(c)) Z direction. In FIG. 11, Residual Motion (mm) is provided along the x-axis and Occurrence Time (%) is provided along the y-axis.

### Part 5

### Tumor Motion Compensation Using Patient Positioning Couch

In this study we have investigated the use of treatment couch for 4D tumor motion prediction and tracking while delivering the radiation beam.

For computer simulation, we have considered mass of the tabletop with payload, m=200 kg; the sampling frequency, v=5 Hz; total simulation time, t=20 sec. It was observed that a couch should move at 3-5 cm/s to compensate motion of the tumor in the range of 1.5-2.0 cm. The velocities in x, y and z directions depend on tumor size, location, and patient specific breathing pattern. Root mean square errors for prediction module in x, y and z directions were 0.267 mm, 0.450 mm and 0.039 mm, respectively (I Buzurovic, K Huang, Y Yu and T K Podder, “A robotic approach to 4D real-time tumor tracking for radiotherapy,” Phys. Med. Biol. 56 (2011) 1299-1318). The overall system error which includes both tracking and prediction error was less than 1 mm.

In this study, we have deployed a model predictive control for an Elekta treatment couch. From the simulation results it appeared that the proposed methods could yield enhanced tracking of the moving tumor. Implementation of the proposed technique can improve real-time tracking of the tumor-volumes for delivering more precise radiation dose at 100% duty cycle while minimizing radiation to the health tissues and sparing the critical organs.

Irregular Motion Simulation

Enhanced Prediction Module

Prediction module (PM) is capable to predict 3D tumor motion for both regular and irregular cycle. Change of the amplitude in each cycle of the normal respiration induced motion is much smaller than that of the irregular respiration induced motion. First 20 seconds is the training period for the PM. The time range from 20^{th} and 40^{th} second is the verification period. Consequently, PM is ready for the simulations after 40 s. The test (or simulation) period is from 40-60 s. Table 3 shows root mean square (RMS) errors of the PM for different time delay.

TABLE 3
RMS ERRORS OF THE PM FOR VARIOUS TIME DELAY

| | | Respiration Type | | | | |
|---|---|---|---|---|---|---|
| | | Normal Respiration | | | Irregular Respiration | |
| | | Prediction Time (ms) | | | | |
| | | 125 | 187.5 | 250 | 156 | 250 |
| PM | RMS error | 0.039 | 0.086 | 0.150 | 0.244 | 0.538 |
| | (cm) | | | | | |
| | % w.r.t. range | 1.09% | 2.43% | 4.25% | 2.80% | 6.17% |

For the specific case, FIG. 2, average errors for prediction module in X, Y and Z directions were, respectively, −0.0092 mm, 0.021 mm and 0.012 mm. RMS errors for prediction module in X, Y and Z direction were, respectively, 0.267 mm, 0.450 mm and 0.039 mm.

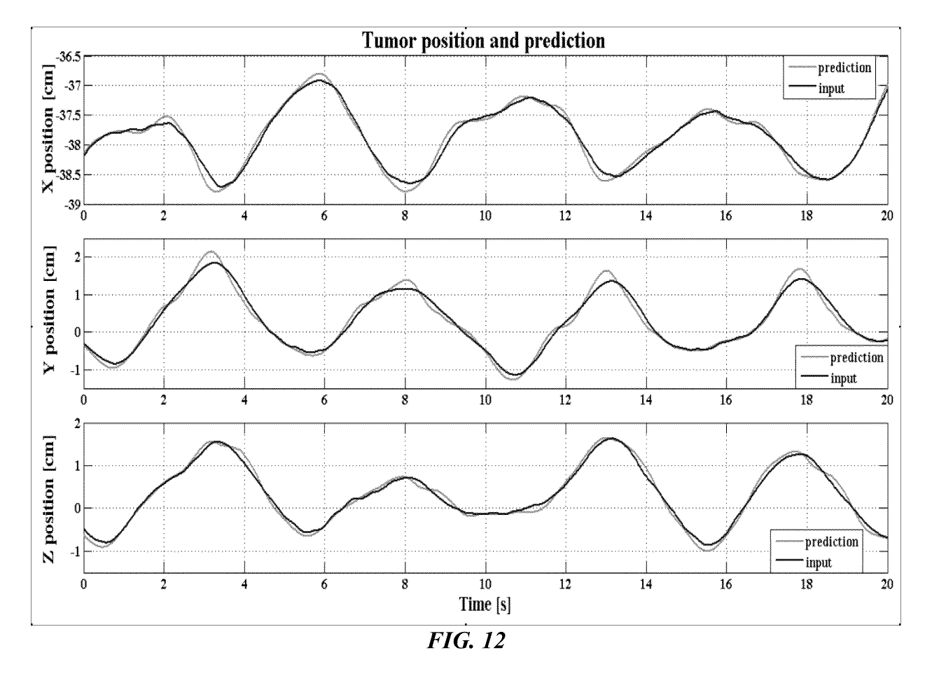

FIG. 12 includes charts of tumor centroid displacement due to cardiac and/or respiratory motion (patient data) Xₘₒₜ and output of the prediction module X_{d} in X, Y and Z directions—real time data. In FIG. 12, Time (s) is provided along the x-axis and Position (cm) is provided along the y-axis.

Simulation Results

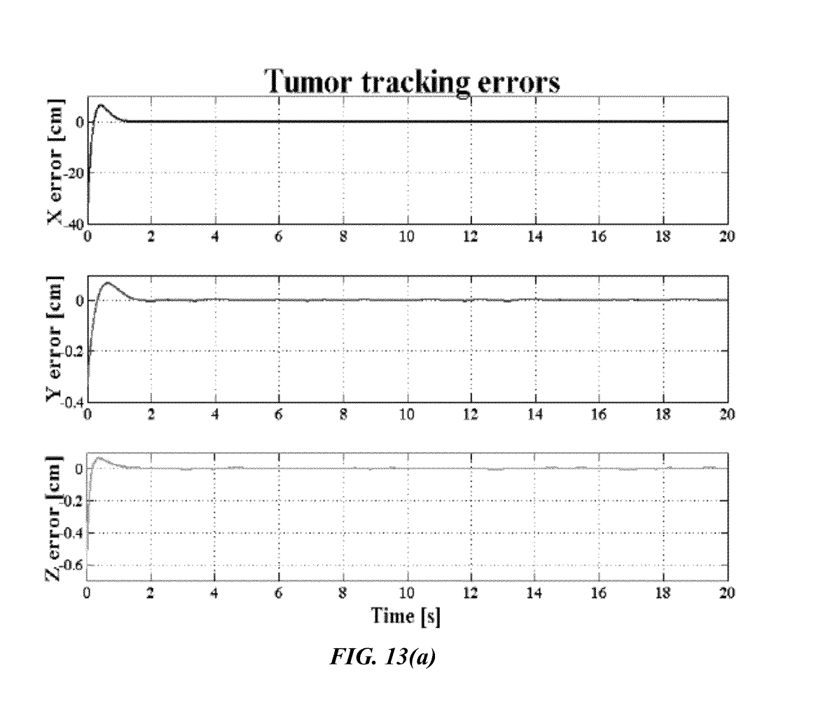

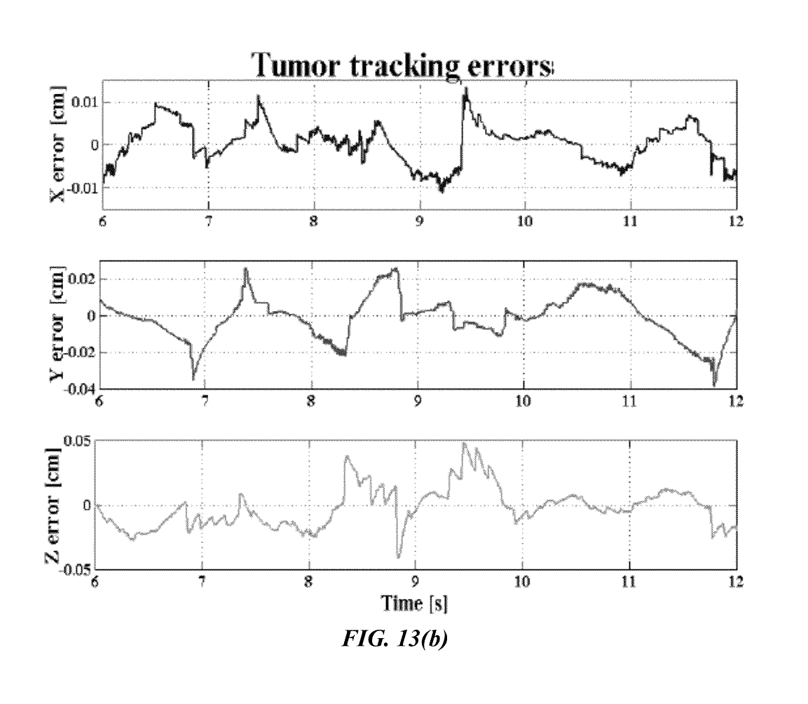

The computer simulation results for ELEKTA Precise Table™ are presented in FIGS. 13-16.

We observed ε_{X}=ε_{Y}=±0.2 mm maximum tracking error for the proposed control system, FIG. 13. In FIG. 13(a) it can be seen that maximum tracking error of the control system in the Z direction was ε_{Z}=±0.5 mm. The tracking error in Z direction appears to be higher due to the mechanical design of the ELEKTA Precise Table™. The system payload is mainly distributed to the Z module as in FIG. 13(a). That is the reason for longer transient process, about 2 s. Nonetheless, after 2 s the system starts the tumor motion compensation with high precision. However, patient weight might have higher influence to the system error in ELEKTA Precise Table™ case.

FIG. 13 is a chart of tumor tracking errors; FIG. 13(a) depicts ε_{X}, ε_{Y}, ε_{Z} for control system in X, Y and Z directions for ELEKTA Precise Table™. FIG. 13(b) depicts the error amplitudes for steady states. In FIG. 13, Time (s) is provided along the x-axis and Error (cm) is provided along the y-axis.

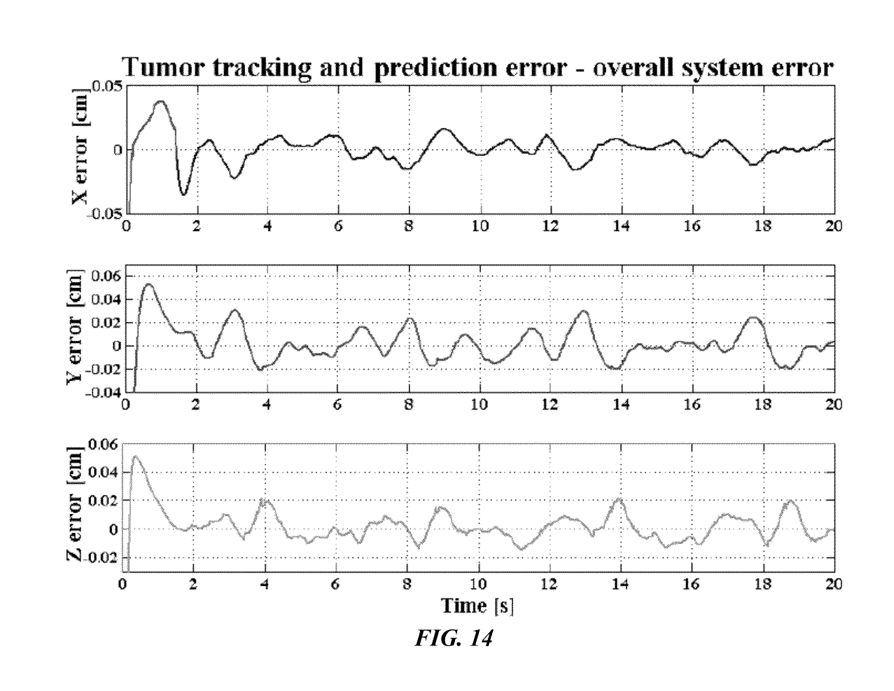

FIG. 14 includes charts of overall system error in X, Y and Z directions for ELEKTA Precise Table™. Overall error includes tracking error and prediction.

FIG. 14 represents overall system error for ELEKTA Precise Table™ which includes both tracking error and prediction error. One can observe that absolute maximum overall error was ε=±0.5 mm and ε=±1 mm for highly irregular respiration. In FIG. 14, Time (s) is provided along the x-axis and Error (cm) is provided along the y-axis.

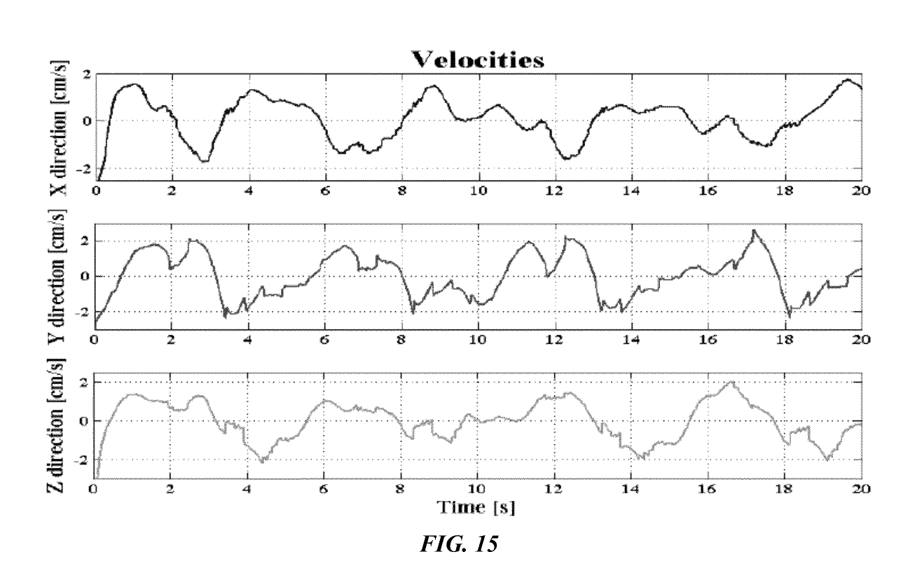

FIG. 15 includes charts of Velocities in X, Y and Z directions for ELEKTA Precise Table™. In FIG. 15, Time (s) is provided along the x-axis and Velocity (cm/s) is provided along the y-axis.

Velocities in three directions depend on patient data. Based on the FIG. 15 it can be concluded that couch should move 3-5 cm/s to compensate tumor motion in FIG. 12. The transient process of about 2 s exists. The rationale is in complicated mechanical design of this table.

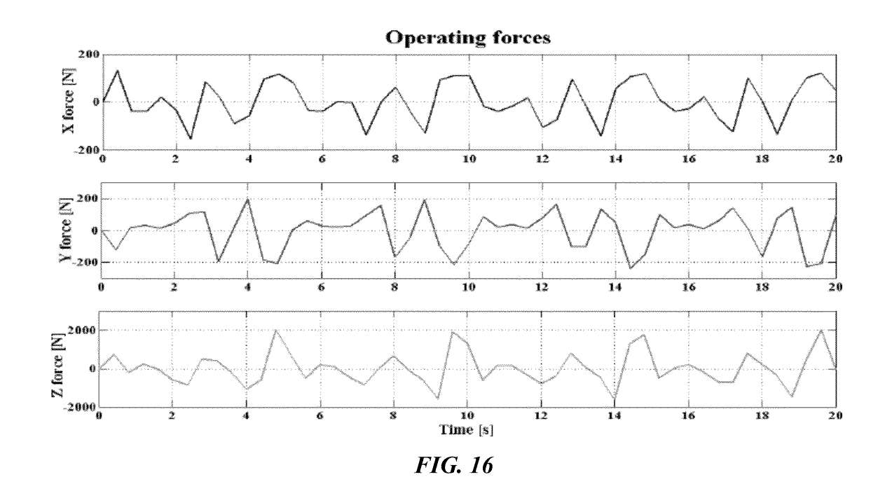

FIG. 16 includes charts of actuation of the ELEKTA Precise Table™ during tumor tracking. In FIG. 16, Time (s) is provided along the x-axis and Force (N) is provided along the y-axis.

It can be observed in FIG. 16 that large force (around 2000N) is necessary to be applied for the vertical motion of the tabletop. Less force has to be applied for both longitudinal and lateral motions, approximately around 200N. This happened due to the heavy payload in the simulation, m=200 kg. Also, heavy mechanical structure of the couch significantly influences the system dynamics. Notwithstanding, it appeared that the dynamics-based controller could track the tumor motion with a high level of accuracy even for the irregular motion.

Discussion

Simulation

It can be noticed that near t=0 FIG. 12 shows very good agreement between the prediction and input motion. Analysing the propagation of the tracking errors during the time for both systems, FIGS. 13-14, a transient time can be noticed. The transient time during the irregular motion is 1.5-2 s for the ELEKTA Precise Table™. It means that the robotic system needs less than 2 s to start tracking with the high level of precision and with a low tracking error (less than 0.5 mm for investigated case and less than 1 mm for highly irregular respiration). Note that PM needs about 40 s to establish and evaluate the motion prediction signal, which makes about 45 s preparation time before tumor has been tracked.

Similarly, observing the velocities for the system, FIG. 15, it can be noticed that the systems start with the tracking immediately. However, the transient time reflect to the velocities as well, so in the initial 1.5 s system is trying to self-regulate the position and velocities, i.e., to minimize tracking error ε using the feedback of the control system. The systems start with the initial velocities equal to zero and accelerate till tumor speed and position is reached.

The design of the parallel robotic platform (HexaPOD couch) supports a high load-carrying capacity. This differs from serial designs, such as robot arms, where the load is supported over a long moment arm, as it is a case for ELEKTA Precise Table™. However, the investigated system appears to have tumor tracking error within 1 mm.

Clinical Significance of Tumor Tracking

Clinical justification of the proposed tumor tracking techniques is presented in the following part.

The purpose of the supporting dosimetry studies was to investigate clinical benefits of tumor tracking and to evaluate changes of treatment volumes when proposed tumor tracking technique is applied. The study includes the evaluation of dosimetric advantages of tumor motion tracking and the irradiation of normal lung and spinal cord. The dosimetric evaluation of tumor tracking was carried out on randomly selected ten patients who were scanned using 4D-CT technique. The 4D-CT phase reconstruction was performed using GE Advantage Workstation software, version AW 4.3-07. The 3D-CRT plans were generated using CMS-XiO4.4. Tissue heterogeneity was corrected for all plans. For each patient eleven dosimetric plans were generated: ten plans for the target volumes contoured at ten breathing phases and one plan for the internal target volume (ITV) generated on average intensity projection (AvIP) studyset. The ITV was defined as a spatial sum of the GTV for each phase. The phase-wise plans were compared to the clinically used ITV-AvIP plans in order to assess dosimetric effects of tumor tracking. PTVs were generated by adding 10 mm margin around GTVs and ITV for both phase-wise plans and ITV-AvIP plans. To analyze data obtained from the dosimetric plans we compared dosimetric parameters including coverage of PTV (D99, D95, D50) volumes of normal lung receiving 5 Gy, 13 Gy, 20 Gy, 30 Gy dose (V5, V13, V20, V30) and D5 of spinal cord for AvIP-based plans with phase-wise tracked plans.

It was observed that during respiratory cycle a tumor volume was changed by up to 20 cm³ depending on tumor size, location, and patient specific breathing pattern. The 3D tumor displacement for all investigated patients were more than 10 mm. Using the proposed active tracking technique it was found that for average tumor motion of 1.5 cm the irradiated PTV was 20-30% less which indicate significant amount of healthy tissue to be spared. Average PTV coverage for all plans was 91.6% of the prescribed dose (PD) for D99, 96.7% for D95 and 104.3% for D50. The average maximum dose was 110% of PD and the mean dose was 103.6% of PD. It was observed that average lung V5, V13, V20, and V30 with tracking technique were about 17.4%, 19.3%, 18.3% and 22.7% lower than the Vxs without tracking, respectively. Calculating dose it was concluded that approximately 20% of healthy lung received 4-8 Gy less dose when the tumor tracking technique was used. Spinal cord was the most important critical organ for the studied lung cases. Dose to the spinal cord (D5) with tracking technique was 17.5% lower compared to that of without tracking. D5 of the spinal cord received approximately 0.5-11 Gy less dose when tumor tracking technique was used; wide variations were observed due to differences in prescribed dose, tumor location and size.

Dosimetric Effect Due to Tracking Errors

It was noticed that total compensation of the motion of thoracic tumors during irradiation may not be possible due to tracking errors. To analyze influence of the tracking error to patient dosimetry, the isocenter of the beams was shifted from the isocenter of the clinical plan by an amount equivalent to the residual motion. The phase-wise study shows that the average differences in the D99 of the PTV and CTV are about 1.37% and 0.21%, respectively. Even in the extreme case (when the respiration cycle was only 3 s, and the amplitudes of tumor motion in the X, Y, and Z directions were 4.1 cm, 5.2 cm, and 4.2 cm, respectively), the difference in the D99 of the CTV was 0.9%. This case, however, is very unlikely to occur. In all other cases, the differences were less than 0.2%. This study reveals that the discrepancy in the delivered doses caused by the tracking error is insignificant for most of the anatomical structures. For example, the average change in the V20 was 0.04%, while the average changes in the D5 of the spinal cord and carina were 0 Gy and 0.14 Gy, respectively.

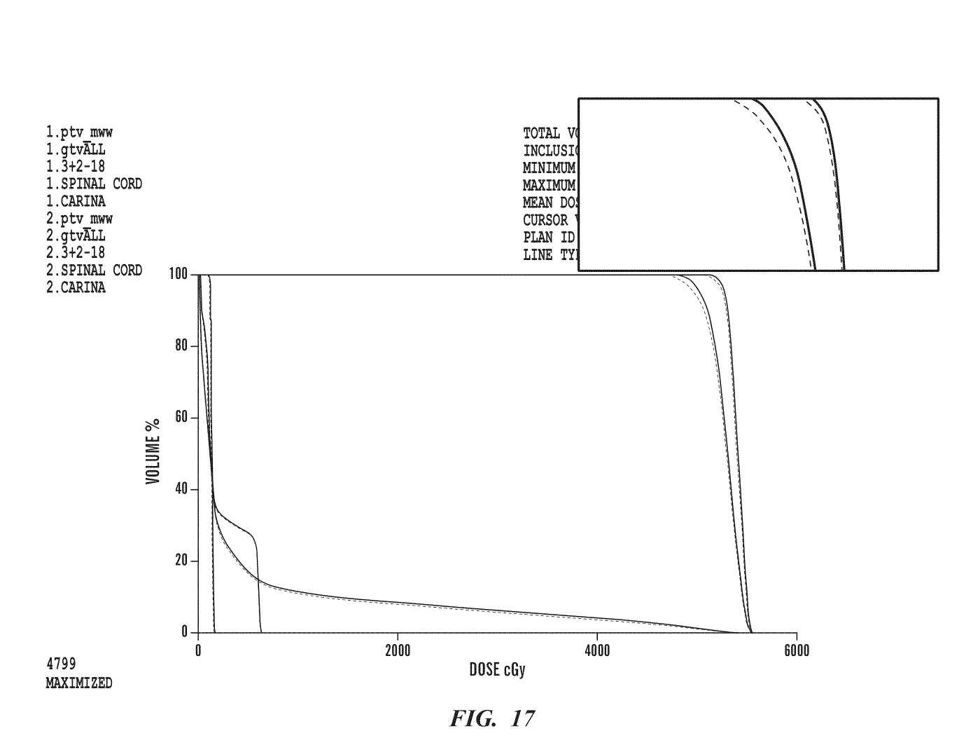

FIG. 17 is a chart of PTV and CTV coverage was not compromised during the tumor tracking procedure (representative case). In FIG. 17, Dose (cGy) is provided along the x-axis and Volume (%) is provided along the y-axis.

### Part 6

### Implementation of Real-Time Tumor Tracking Using a Commercial Couch Modified to Function as a Robotic Couch

Purpose: The purpose of this study was to present a novel method for real-time tumor tracking using a commercial couch modified to function as a robotic couch, and to evaluate tumor tracking accuracy.

Commercially available robotic couches are capable of positioning patients with high level of accuracy; however, currently there is no provision for compensating tumor motion using these systems. Elekta's existing commercial couch (Precise™ Table) was used without changing its design. To establish the real-time couch motion for tracking, a novel control system was developed and implemented. The tabletop could be moved in horizontal plane (laterally and longitudinally) using two Maxon-24V motors with gearbox combination. Vertical motion was obtained using robust 70V-Rockwell Automation motor. For vertical motor position sensing, we used Model 755A-Accu-Coder encoder. Two Baumer-ITD_{—}01_{—}4 mm shaft encoders were used for the lateral and longitudinal motions of the couch. Motors were connected to the Advance Motion Controls (AMC) amplifiers: for the vertical motion, motor AMC-20A20-INV amplifier was used, and two AMC-Z6A8 amplifiers were applied for the lateral and longitudinal couch motions. The Galil DMC-4133 controller was connected to standard PC computer using USB port. The system had two independent power supplies: Galil PSR-12-24-12A, 24 vdc power supply with diodes for controller and 24 vdc motors and amplifiers, and Galil-PS300W72 72 vdc power supply for vertical motion. Control algorithms were developed for position and velocity adjustment.

The system was tested for real-time tracking in the range of 50 mm in all 3 directions (superior-inferior, lateral, anterior-posterior). Accuracies were 0.15, 0.20, and 0.18 mm, respectively. Repeatability of the desired motion was within ±0.2 mm.

Experimental results of couch tracking show feasibility of real-time tumor tracking with high level of accuracy (within sub-millimeter range). This tracking technique potentially offers a simple and effective method to minimize healthy tissues irradiation.

### Part 7

### Effects of Tumor Tracking Errors to the Quality of Radiation Treatment

During radiation therapy, total compensation of thoracic tumor's motion may not be possible due to errors in tracking and prediction techniques. In this study, the dosimetric effects of the residual errors were investigated. Also, the error tolerance level, which would guarantee sufficient quality of the treatment plans, was determined.

The study was performed on 25 patients diagnosed with lung cancer. Eleven plans were generated for each patient, consisting of one clinically accepted initial plan and ten plans with induced tumor tracking errors, using CMS-XIO planning system. The initial plan was used for patient treatments. The other ten plans were generated by shifting the isocenter of the clinical plan to simulate tumor tracking errors from 1 mm up to 10 mm. Tissue heterogeneity was corrected in all cases. The range of the tumor motion was within 2 cm in each direction, and the respiration cycle was 3.5-7.3 s. Plans were compared considering dosimetric parameters including coverage of PTV (D99, D95, D50), volumes of normal lung receiving 5 Gy, 13 Gy, 20 Gy, 30 Gy dose (V5, V13, V20, V30) and D5 of the spinal cord. The initial plans were prescribed to D95 for patient treatments. For the purpose of this study, if the difference in D95 between the initial plan and the plans with induced error was more than 1%, it was considered unacceptable.

It was observed that D95 for 3 mm tracking errors was within a range of −1.09% to +1.98%. Tracking error limit of 3 mm still generated acceptable plans. For the same error limit, the study showed that the average differences in the D99 of the PTV and CTV were within a range of 1.37% and 0.21%, respectively. Even in the extreme case (the respiration cycle is only 3.5 s, and the amplitudes of tumor motion in the X, Y, and Z directions were close to 2 cm), the difference in the D99 of the CTV was 0.9%. In all other cases, the differences were less than 0.71%. This study also revealed that the deviation in the delivered dose caused by the tracking error of 2 mm was insignificant for most of the anatomical structures. For example, in case of spinal cord, the average change in the V20 was 0.04%, while the average changes in the D5 were within 0.34 Gy. Based on these results, it would be reasonable to conclude that even when the overall error during tracking was 3 mm, 89% of the plans were still acceptable. With 2 mm errors, all the plans for all patients (100%) were acceptable. The dosimetric effects of random tracking errors in a range up to 3 mm were negligible.

It can be concluded that during tracking it is not necessary to track respiratory peaks (which appear for short periods of time), and the tumor tracking trajectories can be smoothed. Therefore, the high frequencies of tumor motion can be excluded during real-time tumor tracking.

### Part 8

The commercially available robotic couches are capable of positioning the patient accurately; however, currently there is no provision for compensating the tumor. (I. Buzurovic, K. Huang, Y. Yu, T. K. Podder, “A robotic approach to 4D real-time tumor tracking for radiotherapy”, Phys. Med. Biol. 56, 1299-1318 (2011).)

In the present specification, the implementation procedure for the real-time tracking has been presented together with the experimental results in tumor motion compensation in all three physical dimensions plus time. For that purpose, an existing commercially available treatment couch (Elekta Precise Table™, ELEKTA Ltd., Crawley, UK) was used without changing its design. To establish the real-time couch motion for tracking, a novel control system for the treatment couch was developed and implemented. The basic guidelines for the implementation of novel technology are: a) the treatment couch should maintain all existing standard/regular features for patient setup and positioning, b) the new control system should be used as a parallel system when tumor tracking was demanded clinically, and c) tracking should be performed with single axis motion and/or simultaneously in all three directions of the couch/tumor motion (longitudinal, lateral and vertical). The X direction is defined as the longitudinal table or the superior-inferior patient, Y direction is the lateral table and lateral patient direction, and Z direction is the vertical table and anterior-posterior patient direction.

Dynamic Equations

The first step was to develop the dynamic equations of motion for EPT using energy based Euler-Lagrange formulation (I. Buzurovic, K. Huang, Y. Yu, T. K. Podder, “A robotic approach to 4D real-time tumor tracking for radiotherapy”, Phys. Med. Biol. 56, 1299-1318 (2011)). These equations were essential in developing a dynamics-based coordinated control system. The equations of motion were used to determine the appropriate ranges for proportional, integral, and derivative control gains and the filter parameters.

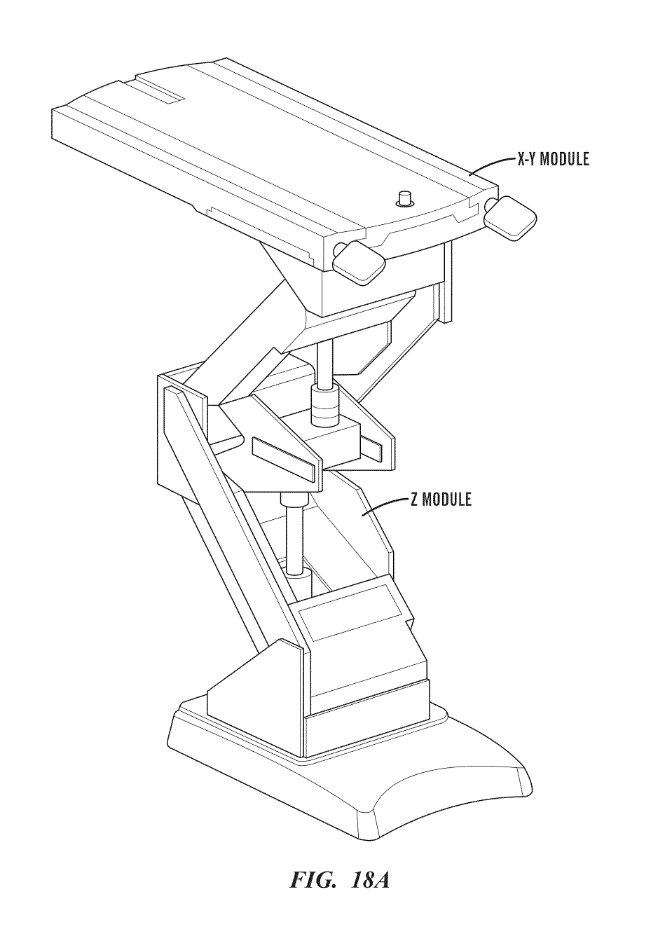

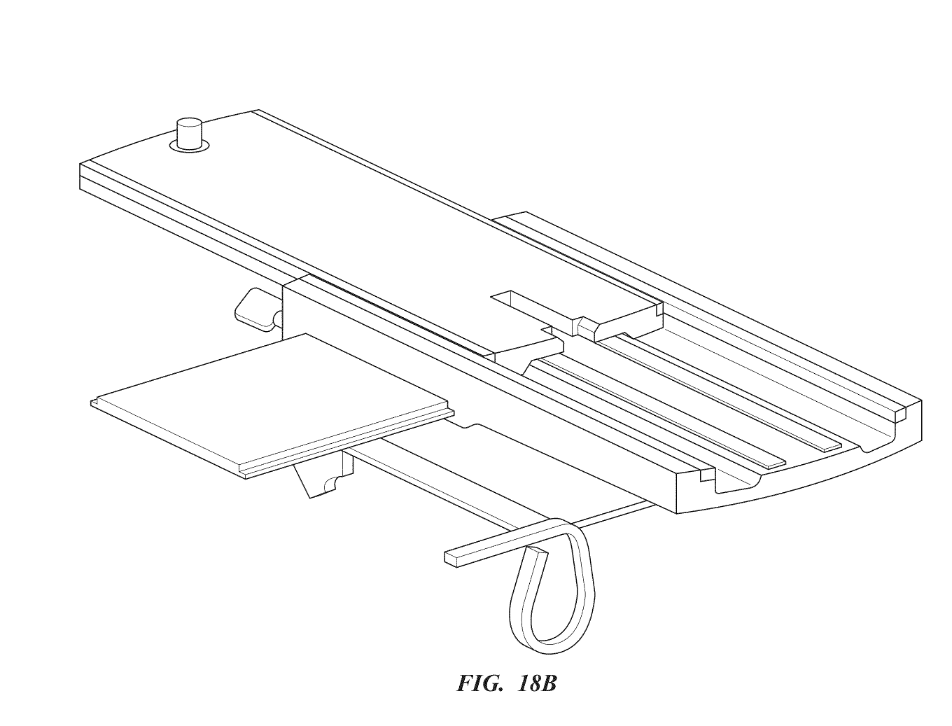

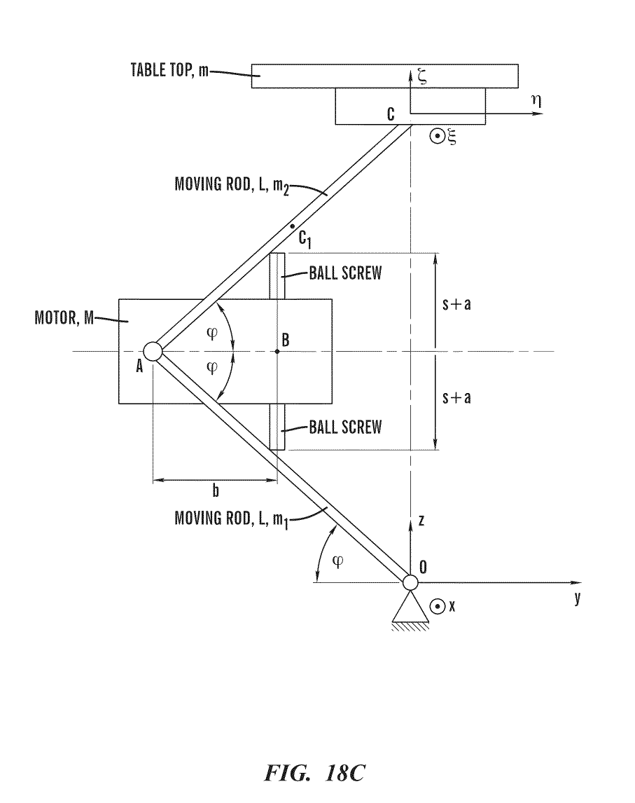

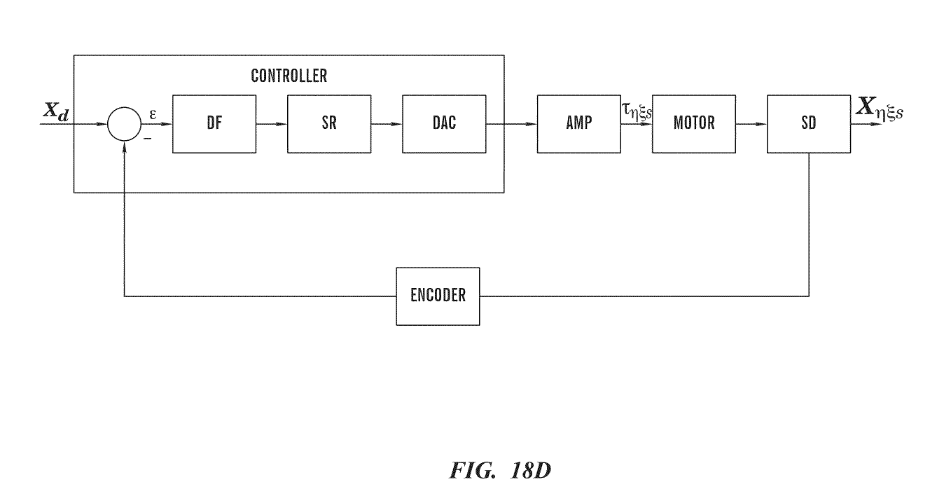

The EPT is an integral part of the system for radiation therapy (FIG. 18(a)).

FIG. 18 is a schematic view of ELEKTA Precise Table™. FIG. 18(a)-(b) are internal isometric views and FIG. 18(c) is a system model. Vertical movement in s direction is achieved by motor installed in the holder A. Tabletop movement in ξ and η directions are achieved by two motors sitting below the tabletop.

EPT consists of a 2 degree-of-freedom (DOF) tabletop and a 1 DOF vertical lift. The vector of generalized coordinates q for the EPT was chosen as follows: vertical motion of the table −s, relative motion of the tabletop −ξ and η. Consequently, q=(sξη)^{T}. The schematic view of the system is shown in FIG. 18. Referring to FIG. 18, a fixed coordinate system was assigned as (x y z) at the center O of the point where the table is connected to the floor. The moving coordinate system (ξηζ) at the center C is attached to the tabletop. The vertical direction motor (mass M) drives the ball screws, which are responsible for the vertical motion of the mechanism in z direction with respect to Oxyz coordinate system. The end of the upper moving rod (length L, mass m₂) is fixed to a tabletop holder (FIG. 18(c)). Both the lower and upper moving rods are of the same length.

The tabletop (mass m, including load) effectuates a plane motion in C_{ξηζ} coordinate system. The motion of the mechanism is analyzed with respect to a fixed coordinate system Oxyz. The tabletop moves ξ in and η directions with respect to the coordinate system C_{ξηζ}. Coordinate system C_{ξηζ} is fixed to a table holder. The table holder vertical motion induces changes of the generalized coordinate s. Lengths a and b are geometric characteristics of the mechanism. Angle φ is variable and its change implies changes of the generalized coordinate s.

In the following section, only a limited number of key-equations have been presented. The geometric relations and velocities used for the following derivation as well as more detailed derivations were presented in other publications. (I. Buzurovic, K. Huang, Y. Yu, T. K. Podder, “Tumor Motion Prediction and Tracking in Adaptive Radiotherapy”, Proc. of IEEE Int. Conf. on Bioinformatics and Bioeng. 273-278 (2010); I. Buzurovic, K. Huang, Y. Yu, T. K. Podder, “A robotic approach to 4D real-time tumor tracking for radiotherapy”, Phys. Med. Biol. 56, 1299-1318 (2011).)

The Lagrangian function of dynamic systems can be expressed as:

L=kinetic energy(T)−Potential energy(π) (1)

The general form of dynamic equations is

$$\frac{d}{d t} ( \frac{\partial L}{\partial \dot{q}} ) - \frac{\partial L}{\partial q} = τ \qquad ( 2 )$$

where qεRⁿ, and τ is the generalized force (or torque) applied to the system through the actuators. The final expression for the potential energy is:

$$Π = \frac{( {m}_{1} + 3 {m}_{2} + 2 M + 4 m ) gL ( s + a )}{2 \sqrt{{b}^{2} + {( s + a )}^{2 }}} . \qquad ( 3 )$$

The total kinetic energy of the system is:

T=T_{OA}+T_{AC}+Tₘₒₜₒᵣ+Tₜₜ, (4)

where the kinetic energies of the moving rods OA and AC, the motor at point A, and the tabletop were denoted as T_{OA}, T_{AC}, Tₘₒₜₒᵣ and Tₜₜ, respectively. The force which is responsible for the translational motion of the axis is ξ denoted by τ_{η}. The force which is responsible for the translational motion of the axis η is denoted by τ_{η}.

Combining equations (1-2) with (3) and (4), the general equations of motion for EPT were as follows:

$$\begin{matrix}m \ddot{ξ} = {τ}_{ξ} \\ m \ddot{η} = {τ}_{η} & ( 5 ) \\ ( \begin{matrix}\begin{matrix}\frac{{b}^{2} {L}^{2}}{3 {( {b}^{2} + {( a + s )}^{2} )}^{4}} \\ ( \begin{matrix}- 2 ( a + s ) ( \begin{matrix}{a}^{2} ( 3 M + {m}_{1} + {m}_{2} ) + \\ {b}^{2} ( \begin{matrix}18 m + 3 M + \\ {m}_{1} + 10 {m}_{2}\end{matrix} ) + \\ ( 3 M + {m}_{1} + {m}_{2} ) s ( 2 a + s )\end{matrix} ) {\dot{s}}^{2} + \\ \begin{matrix}( {a}^{2} + {b}^{2} + 2 as + {s}^{2} ) \\ ( \begin{matrix}\begin{matrix}{a}^{2} ( 3 M + {m}_{1} + {m}_{2} ) + \\ {b}^{2} ( 12 m + 3 M + {m}_{1} + 7 {m}_{2} ) +\end{matrix} \\ ( 3 M + {m}_{1} + {m}_{2} ) s ( 2 a + s )\end{matrix} ) \ddot{s}\end{matrix}\end{matrix} ) +\end{matrix} \\ \frac{{b}^{2} gL ( 4 m + 2 M + {m}_{1} + 3 {m}_{2} )}{2 {( {b}^{2} + {( a + s )}^{2} )}^{3 / 2}}\end{matrix} ) \frac{h}{4 π} = {τ}_{M} & \end{matrix}$$

The equations of motion (5) fully describe the dynamical behavior of the EPT. In the following part, it will be denoted as System Dynamics (SD).

Control Methodology

The purpose of control methodology is to allow both the modes of operations, i.e., use of the table for patient positioning and tumor tracking. During patient positioning the couch should maintain all standard functions as in regular use. Additionally, during the radiation treatment, the couch should perform real-time tracking of the tumor. By the term real-time tracking we refer to tracking in all three dimensions together with temporal variation, which is 4D tracking.

The block diagram and control methodology are presented in FIG. 18(d).

FIG. 18(d) depicts functional elements of tumor tracking control system; DF-digital filter. SR=ZOH signal reconstruction, DAC digital to analog converter. The ZOH, or zero-order-hold, represents the effect of the sampling process, where the motor command is updated once per sampling period. The DAC or D-to-A converter converts a 16-bit number to an analog voltage.

The controller DMC-41×3 (Galil 3 Axis Controller, Galil Motion Control, CA with 500 mA sourcing outputs) provides two communication channels: a high speed 100BaseT Ethernet connection and a USB programming port. The controllers allow for high-speed servo control up to 15 million encoder counts/sec and step motor control up to 3 million steps per second. The controller eliminates jerk by programmable acceleration and deceleration with profile smoothing. These characteristics allow adjusting the system for both the best patient comfort during tracking and accurate motion trajectory tracking.

The digital filter (DF) has three elements which are responsible for the treatment couch control. These elements are the proportional-integral-derivative (PID), low-pass and a notch filter. The dynamic-based controller was proposed and explained in details in another publication. (I. Buzurovic, K. Huang, Y. Yu, T. K. Podder, “A robotic approach to 4D real-time tumor tracking for radiotherapy”, Phys. Med. Biol. 56, 1299-1318 (2011).) To reduce any steady-state errors, an integral control part was also incorporated into equation (6). Thus, the final control equation becomes,

$$\ddot{ɛ} + {K}_{D} \dot{ɛ} + {K}_{P} ɛ + {K}_{I} {\int }_{0}^{t} ɛ d t = 0 \qquad ( 6 )$$

where K_{D}, K_{P} and K_{I} are the derivative, proportional and integral gains. Equation (6) ensures asymptotic decay of the transient errors as well as the reduction of the steady-state errors. The gains were calculated to cancel the resonance effect during tracking by placing the complex zeros on the top of resonance poles of the system in FIG. 18(d). For instance, low-pass and PID elements have transfer function (7)

W(s)=(P+zD+I/z)a/(z+a) (7)

In expression (11), z is the time parameter in the Laplace domain, and P=K_{P}, D=TK_{D}, I=K_{I}/T, a=1/T In(1/B), T is the sampling period, and B is the appropriate pole setting which guarantee the system stability during tracking.

In the following section two motion compensation techniques have been described. The first one is tumor tracking without knowing the tumor position in advance (tracking mode), and the second is the adaptive contouring mode, when the trajectory is known before the treatment starts. In another word, if the motion trajectory was obtained in a real-time during patient treatment using external/internal marker, the controller works in the tracking mode. The adaptive contouring mode is the one when the motion trajectory was defined prior the treatment (for instance, using 4D CT).

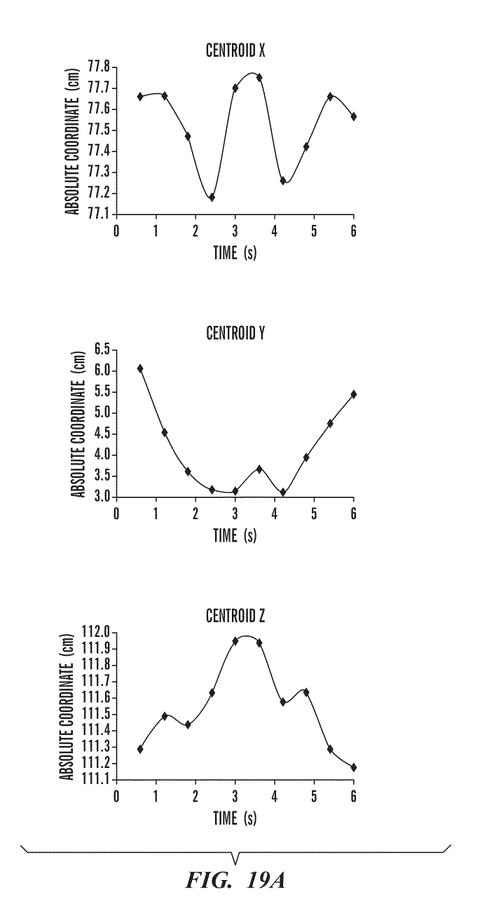

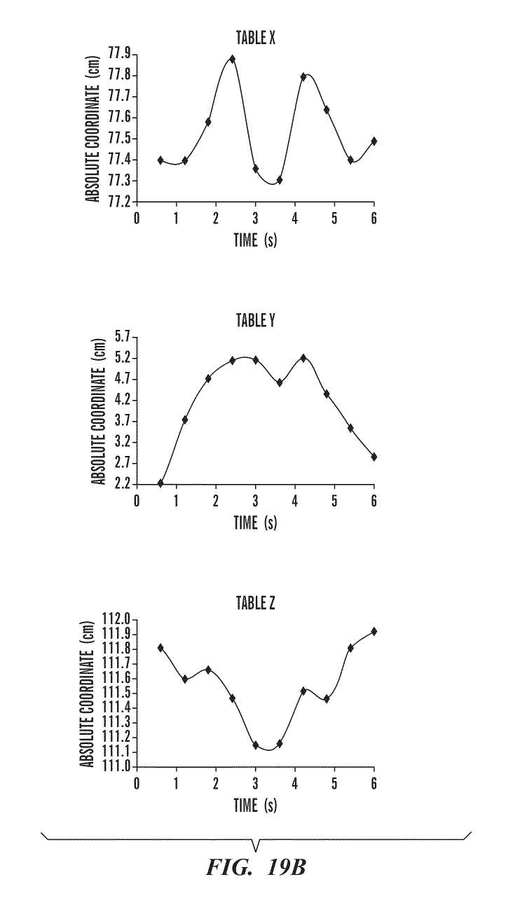

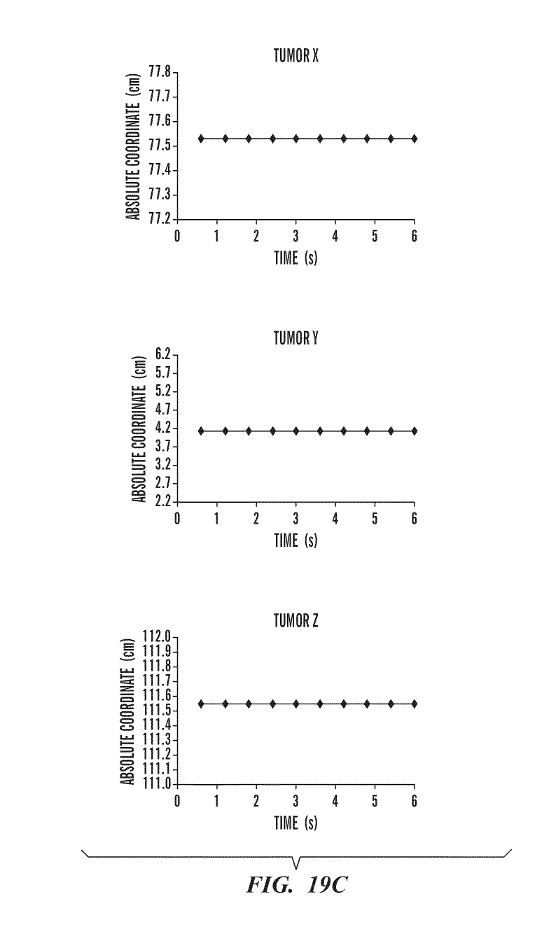

For the online tumor tracking, the controller should be placed in the tracking mode to support changing position of the target volumes (absolute position change) during the treatment. The controller then calculates a new trajectory based upon the new target and acceleration, deceleration, and speed parameters that have been set. The controller updates the position information at the rate of 1 ms. The controller generates a profiled point for every other sample, and linearly interpolates one sample between all profiled points. Based on the tumor velocity and position, the controller either sends the signals to continue in the direction to where it is heading, or changes the direction where it moves, or decelerates to a stop. The position tracking mode is suitable in the case when the internal markers give the real-time position during motion compensation and tracking. In that case, the proposed system is able to generate the robotic couch trajectory on the fly. The implemented tracking mode allows arbitrary motion profiles to be defined by position, velocity and time for the individual motion trajectories. By specifying the target position, velocity and time to achieve the parameters the user has control over the velocity profile of the system motion. Taking advantage of the built in buffering the user can create virtually any profile and consequently, the system is able to perform tracking for variety motion profiles. Furthermore, using one of the described tracking modes and the control strategy, it is possible to program any type of motion for successful tracking. The controller interpolates the motion profile between the subsequent positions using a third-order polynomial equation, which is an inbuilt interpolation method of the control card used. The decomposed motion of the tumor centroid, robotic table and relative tumor motion position were presented in FIG. 19.

FIG. 19(a) is a chart of the motion of the decomposed tumor centroid, FIG. 19(b) is a chart of the motion of the table and FIG. 19(c) is a chart of the motion of the tumor relative motions in absolute coordinate system in the tracking mode. Data show one representative case for breathing cycle of 6 s in X, Y and Z direction. The trajectories represent real patient tumor motion. In FIG. 19, Time (s) is provided along the x-axis and Absolute coordinate (cm) is provided along the y-axis.

The couch motion of each axis does not start unless the appropriate command is given from the control computer interface. The command ensures that all axes start the motion simultaneously. However, it is not necessary that all axes have the same time stamp, i.e., for demanding motion trajectories, the time delay in any direction can be implemented if needed. The tracking system was designed to control the errors using the encoders. The controller then performs in the following steps: the motion can be maintained or fully stopped, and with the proper interface with the linear accelerator, the radiation beam can be interrupted. The velocity profiles can be smoothed in order to reduce the couch vibrations.

System Integration

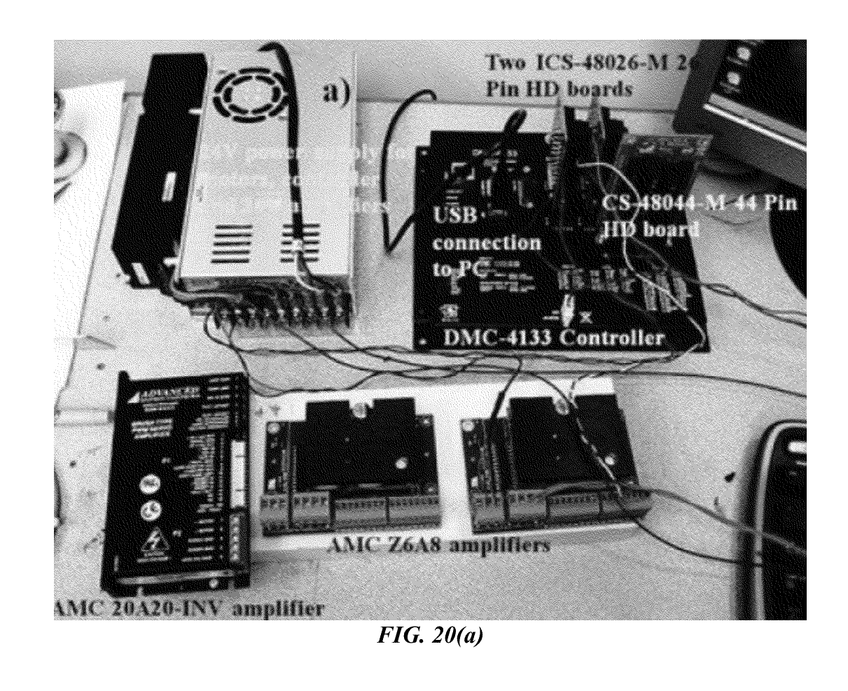

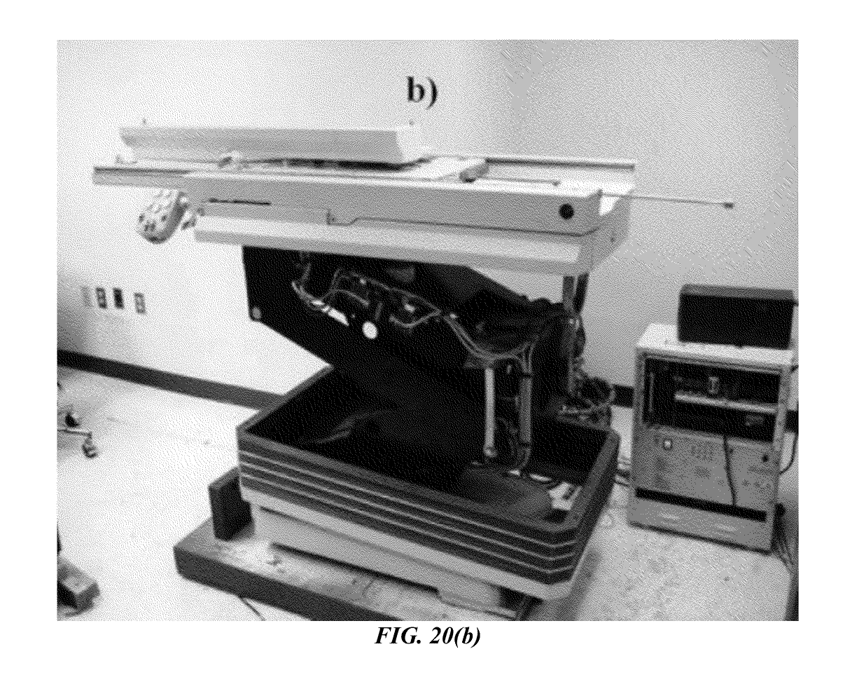

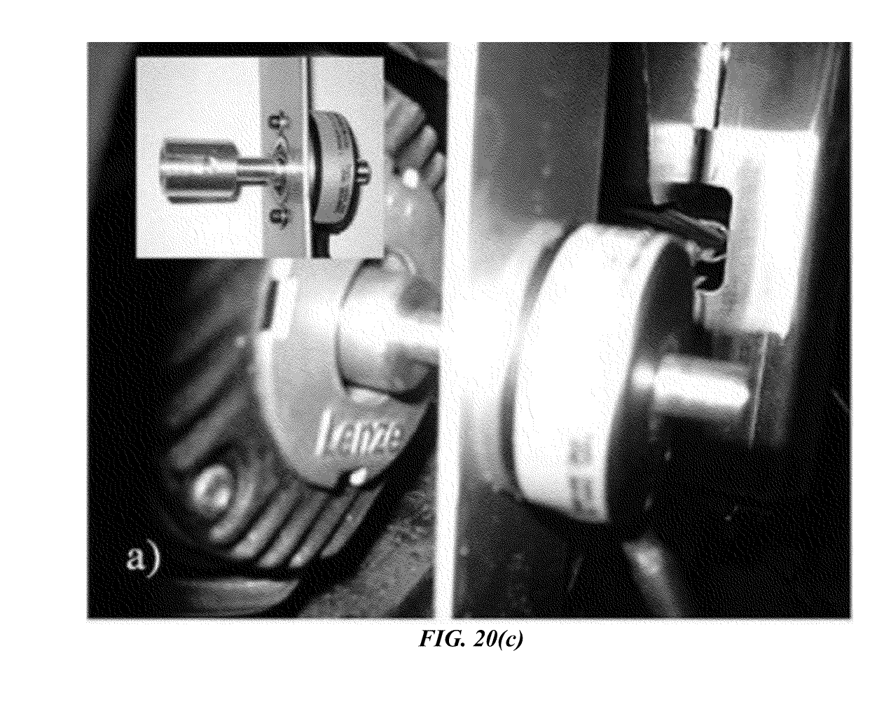

To apply the dynamic-based control of the system to the EPT, system dynamics equation (9) has been used. The tabletop can move in the horizontal plane (laterally and longitudinally) using two Maxon 24V motors with gearbox combination. The vertical motion is obtained using a robust 70V Rockwell Automation motor. To obtain the exact position of the table, the Baumer ITD 01 (4 mm shaft) encoders for X and Y motions were used, and the Model 755A Accu-Coder encoder for Z motion was used. The encoders were connected to the Advance Motion Controls amplifiers (AMC 20A20-INV amplifier for Z direction, and two AMC Z6A8 amplifiers for X and Y direction) to the Galil DMC-4133 controller for all 3DOF. The system has two independent power supplies: the Galil PSR-12-24 12A, 24 vdc power supply with diodes for controller, 24 V motors and amplifiers, and the Galil PS300W72 72 vdc power supply for vertical motion. The controller consisted of a new control algorithm developed for closed-loop control of the system using the position and velocity feedback. The equipment (FIG. 20(a)) has been mounted on the commercially available EPT (FIG. 20(b)).

FIG. 20(a) depicts control system integration parts, and FIG. 20(b) depicts ELEKTA Precise Table™ robotic treatment couch—experimental setup with reference coordinate system.

The connection of the horizontal plane encoders to the controller was obtained by a 26 pin HD D-Sub female connector, whereas for the vertical motion encoder a CS-48044 M 44 pin connector was used. The AMC 20A20-INV amplifier for the Rockwell Motor is designed to drive brush type DC motors at a high switching frequency. The drive is fully protected against over-voltage, under voltage, over-current, over-heating and short-circuits across motor, ground and power leads. The X and Y encoders were mounted using the flexible mounting system which is tolerant to axial misalignment or radial shaft run-out. The Z encoder was mounted on the vertical Rockwell motor using an in-house made connector, as shown in FIG. 20(c).

FIG. 20(c) depicts installation of the encoder to vertical lift motor. The insert of FIG. 20(c) shows the adapter and holder for encoder. FIG. 20(d) depicts installation of the encoder for longitudinal couch motion (X direction). The insert of FIG. 20(d) represents the encoder mounting to the existing motor.

Experimental Setup

Couch Performance Test

To evaluate the performances of the modified treatment couch we investigated the mechanical characteristics of the system such as system resolution, repeatability, accuracy, and tracking using the maximum couch speed (45 mm/s). For these tests, the encoders reading, high resolution camera and vernier caliper were used. The couch was moved in the predefined positions using different speeds up to the maximum speed for the motion in all three directions. (ELEKTA Ltd., Crawley, UK, “Precise Treatment Table—Corrective Maintenance Manual”, 2002.) The tests were performed using the nominal system resolutions of 1/3600 mm in X and Y directions and 1/1200 mm in Z direction.

Tumor Tracking Test—Mechanical

For simultaneous tracking in all three dimensions, the MotionSim XY/4D (Sun Nuclear Corporation, Melbourne, Fla.), a motion phantom was used. The maximum speed of the motion phantom was 50.8 mm/s in X and Y directions, and 12.7 mm/s in Z direction. The approach was to use a phantom to simulate tumor motion, and to use the couch to compensate it. The motion phantom is designed to have independent 2 DOF X and Y motions, and 1 DOF vertical motion. Additionally, we used the AlignRT-3D imaging solution for the patient setup and real time tracking (VisionRT, London, UK) to evaluate the 4D motion, and to independently check the motion in Z direction. The AlignRT was used via surface imaging of the motion phantom.

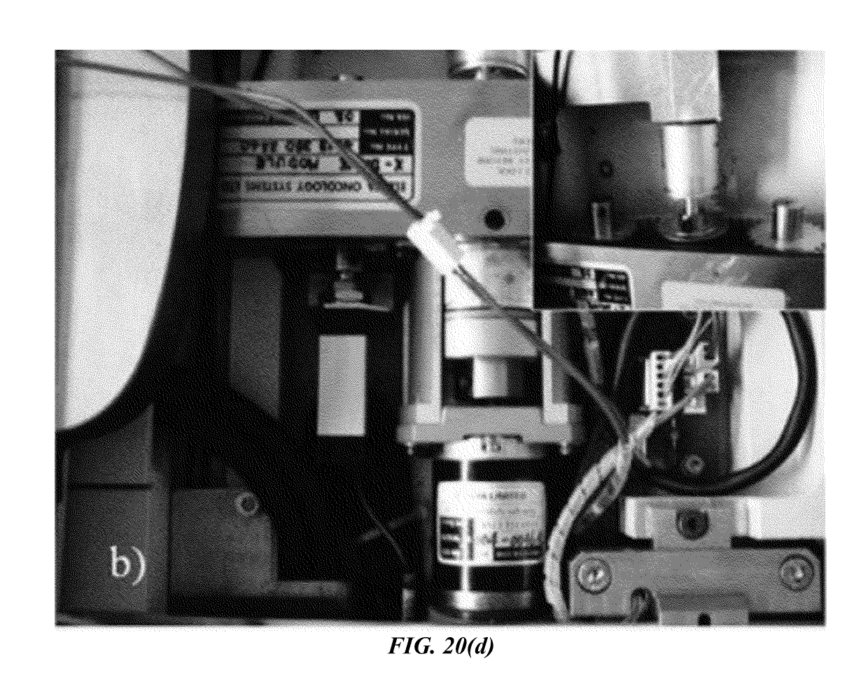

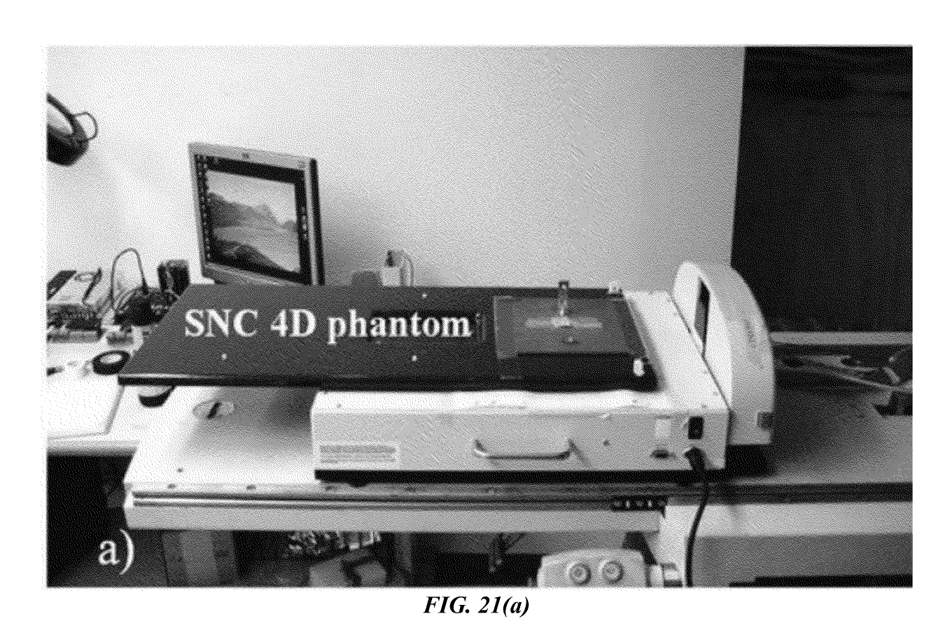

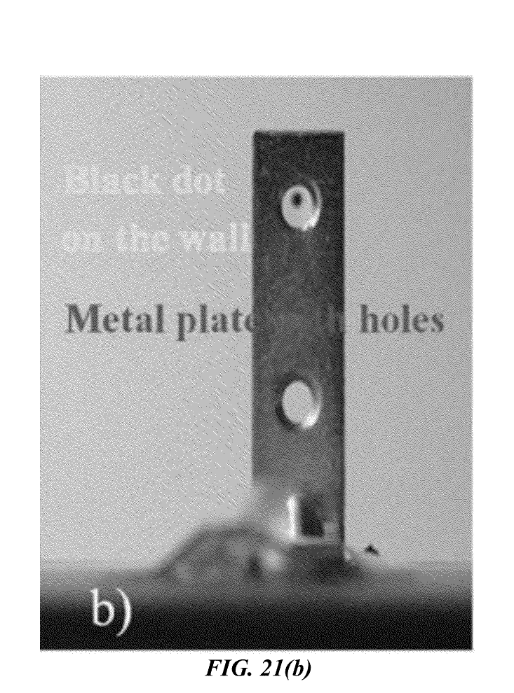

The 4D MotionSim phantom was placed on the top of the table, shown in FIG. 21(a), and was programmed to move simulating the tumor motion. The metal plate with 2 mm holes was installed on the top of the 4D phantom. The camera was fixed from the side to record the black dot (FIG. 21(b)) on the wall which appeared stationary and visible during the time when of both the table and phantom moved.

FIG. 21(a) depicts an experimental setup with a Sun Nuclear programmable 4D phantom on the top of the table, and FIG. 21(b) depicts the metal plate with the hole fixed on the top of the 4D phantom.

Consequently, to keep the absolute position of the dot stationary/stable, the table was move in the opposite direction; as if to create the scenario where the tumor appears stationary with respect to the radiation beam. The images were then analyzed for evaluating tracking performance. The experiments were performed taking the system latency of 100 ms into account.

Tumor Tracking Test—Dosimetry with External Radiation Beam

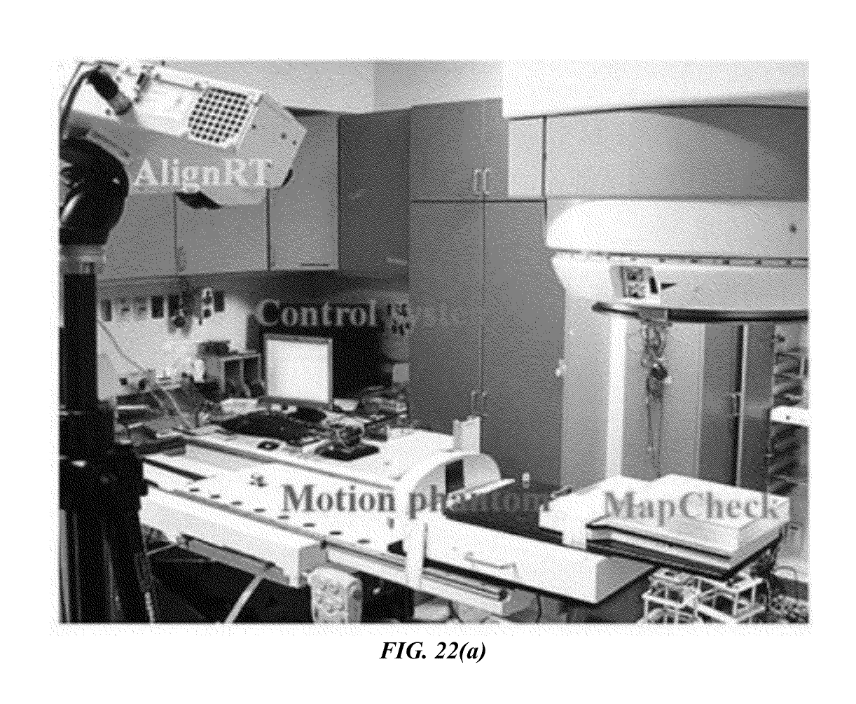

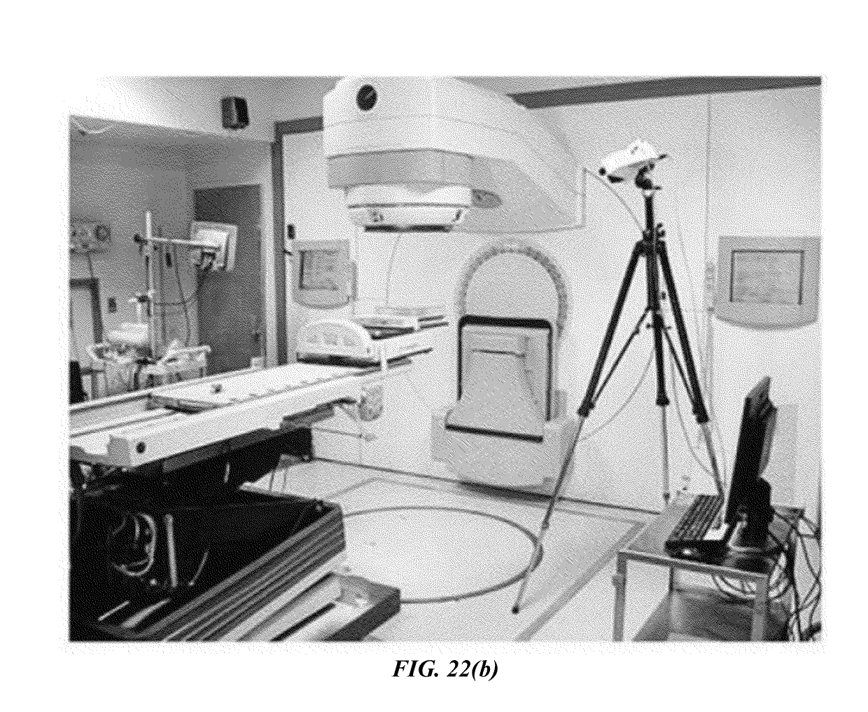

To investigate the feasibility of real-time tracking in the clinical setting, the existing treatment table was replaced with our experimental table (FIG. 22).

FIGS. 22(a)-(b) depict an experimental setup of the tumor motion compensation system.

The motion phantom was installed on the top of the table, and the MapCheck (Sun Nuclear Corporation, Melbourne, Fla.) was placed and secured on the top of the motion phantom. The motion of the table and phantoms was monitored using the AlignRT imaging system, as shown in FIG. 22(a)-(b). The couch was programmed to countermove relative to the motion phantom so the MapCheck appeared stationary with respect to the radiation beams. The first tumor motion trajectory was as shown in FIG. 19, with 6 s breathing cycles and a maximum breathing extent of 3 cm in Y direction. An additional tumor motion trajectory with a breathing cycle of 7.5 s and 2 cm maximum motion in X direction were also considered. The tumor motion trajectories were obtained from 4D CT scans of real patients. The two different lung plans (a 3D conformal and an IMRT plan) were delivered first in a traditional manner, i.e., without compensating for tumor motion and then with the tumor motion compensation.

Couch Performance Test

It was noticed that with heavy load (100 kg) on the couch there were motion dead-zones of 0.1 mm in X and Y and 0.2 mm in Z direction. However, this fact did not influence the overall system performances. The accuracies for the linear range of motion of 200 mm in X, Y and Z were 0.10 (SD=0.10), 0.10 (SD=0.10) and 0.12 (SD=0.13) mm, respectively. The repeatability test demonstrated the same level of accuracy for ten consecutive motions in the positive and negative direction. The system was able to change the velocity successfully from 1 mm/s to 45 mm/s and back from 45 mm/s to 1 mm/s without motion interruptions within 4 s with maximum load. Based on these tests, it can be concluded that the modified treatment couch can potentially perform the tracking task.

Tumor tracking test—mechanical

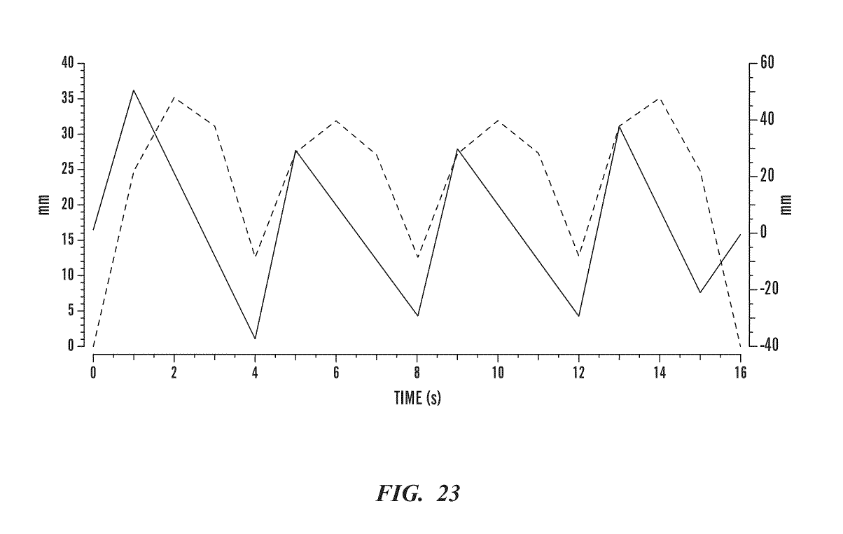

The system was tested for real-time tracking in the range of 50 mm in all 3 directions (superior-inferior, lateral, anterior-posterior). The accuracies were 0.12, 0.14, and 0.18 mm, respectively. The repeatability of the desired motion during trajectory tracking was within ±0.2 mm. The test motion profile of the table in X and Y directions are shown in FIG. 23.

FIG. 23 depicts table motion in X and Y direction for the tumor tracking test. In FIG. 23, Time (s) is provided along the x-axis and Position (mm) is provided along the y-axis.

It was observed that the relative motion of the metal plate (FIG. 21) was successfully canceled by the longitudinal and lateral motions of the couch, even in the transition moments when the direction, motion amplitude and velocity were changed. Using the AlignRT system, it was observed that the vertical lift tracked the predefined trajectory with a maximal error of ±0.3 mm.

Tumor Tracking Test—Dosimetry with External Radiation Beam

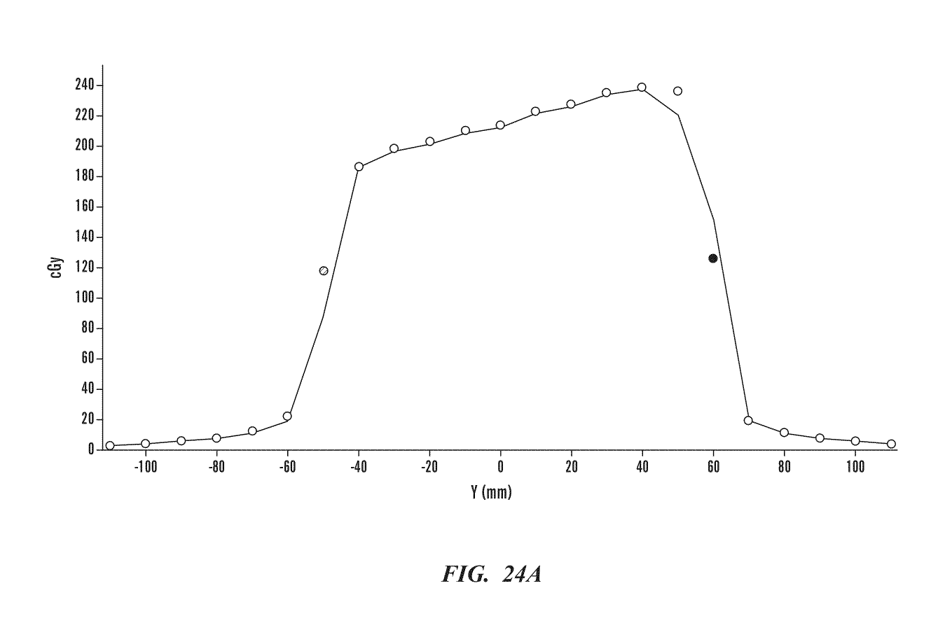

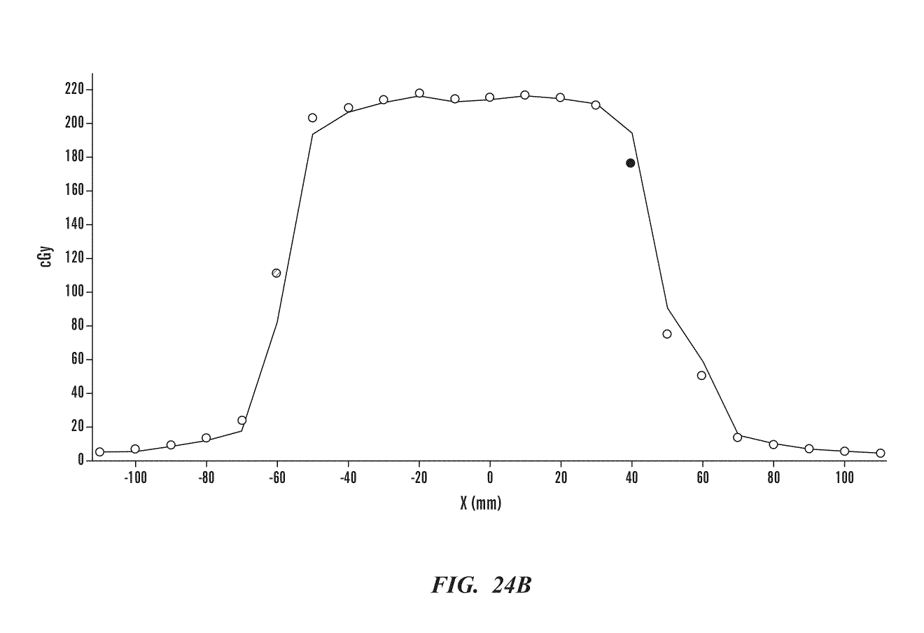

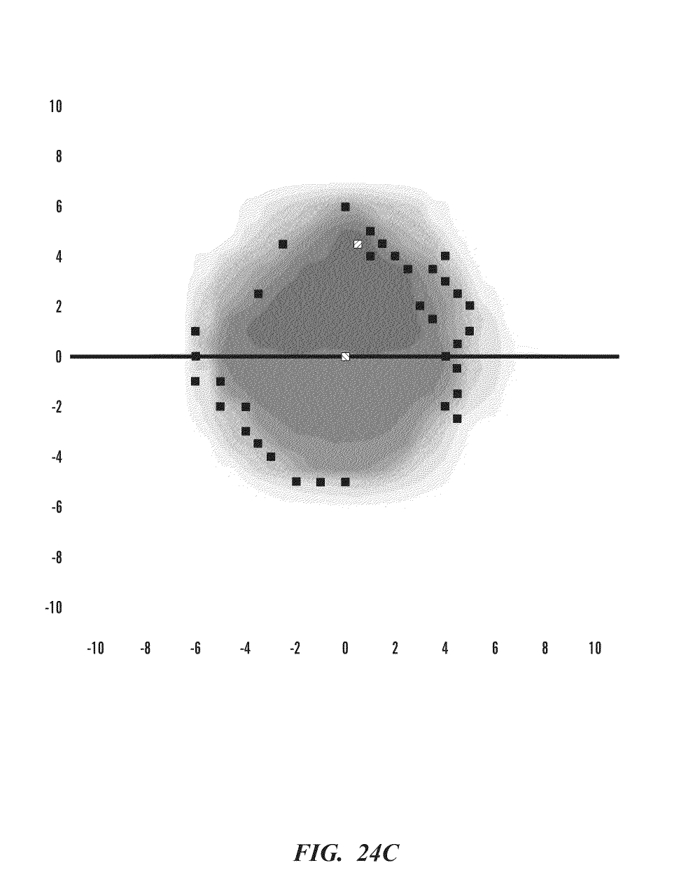

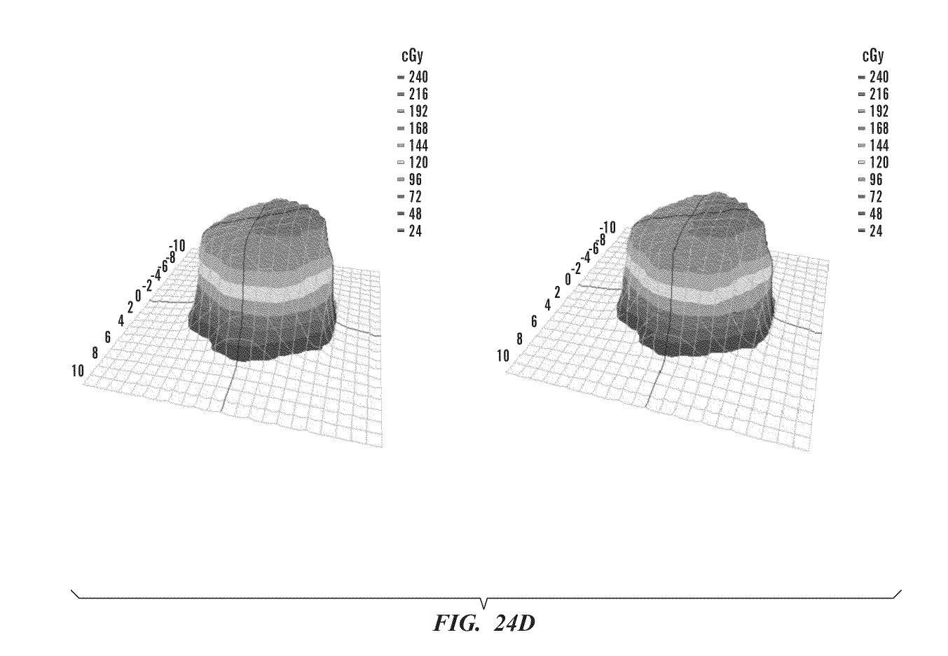

Using the setup described in above, a reference plan without the motion, i.e., both the tumor (a mass in the 4D phantom) and the couch were stationary, was initially delivered for a lung 3D conformal plan. Later, additional plans were delivered for two motion trajectories, shown in FIG. 19. The central axis (CAX) dose for the reference plan was 213.75 cGy, whereas the CAX doses for other two plans were 213.24 cGy and 210.94 cGy (0.21% and 1.19% difference). The doses in inplane and crossplane profiles were in the same range as in the CAX doses (FIG. 24(a)-(b)).

FIG. 24 depicts comparison of the stationary plan with the tracking one, where FIG. 24(a) depicts inplane profile, FIG. 24(b) depicts crossplane profile, FIG. 24(c) depicts passing criteria is critical in the high gradient region and FIG. 24(d) depicts 3D dose profile for both plans. In FIG. 24(d), the Dose (cGy) ranges from 24 to 240 cGy, where the region corresponding with 24 cGy is closest to the bottom of the Figure and where the region corresponding with 240 cGy is closest to the top of the Figure. Yellow dots denote the dose differences within 1%; blue and red dots denote dose differences higher and lower than 1% for the specific profile. In FIG. 24(a), Y-axis motion (mm) is provided along the x-axis and Dose (cGy) is provided along the y-axis. In FIG. 24(b), X-axis motion (mm) is provided along the x-axis and Dose (cGy) is provided along the y-axis. In FIG. 24(c), X-axis motion (mm) is provided along the x-axis and Y-axis motion (mm) is provided along the y-axis. In FIG. 24(d), X-axis motion (mm) is provided along the x-axis, Y-axis motion (mm) is provided along the y-axis and Dose (cGy) is provided along the z-axis.

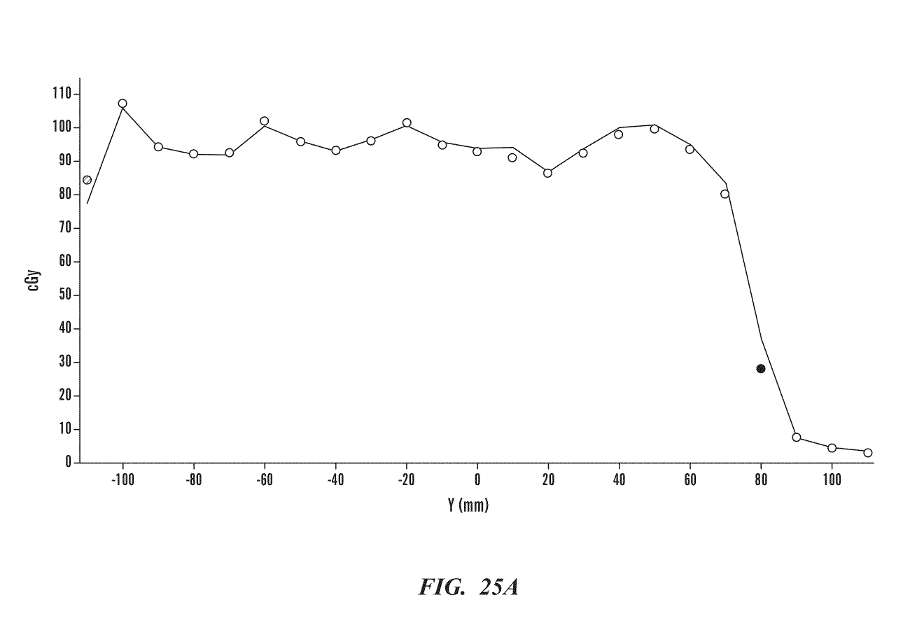

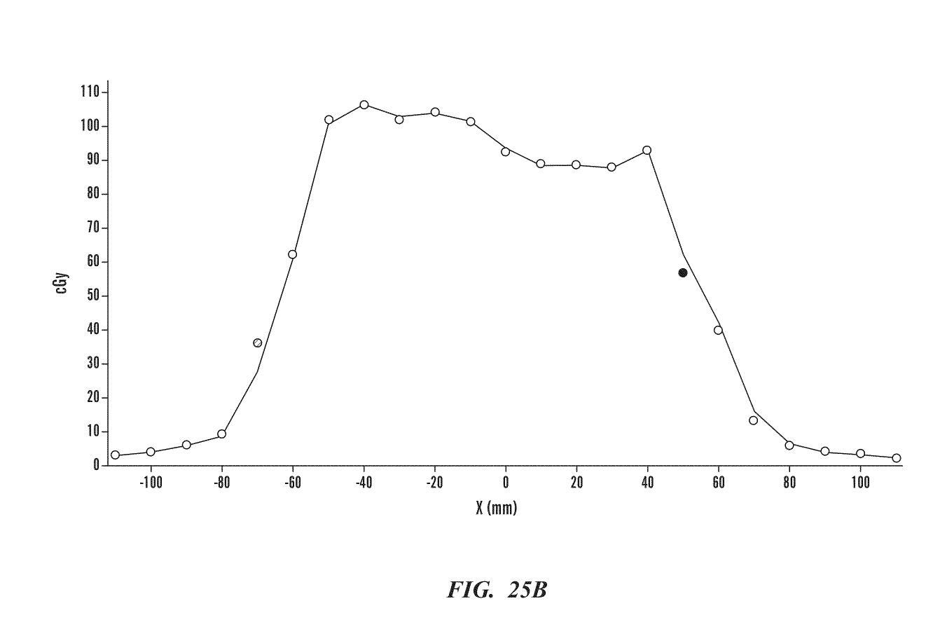

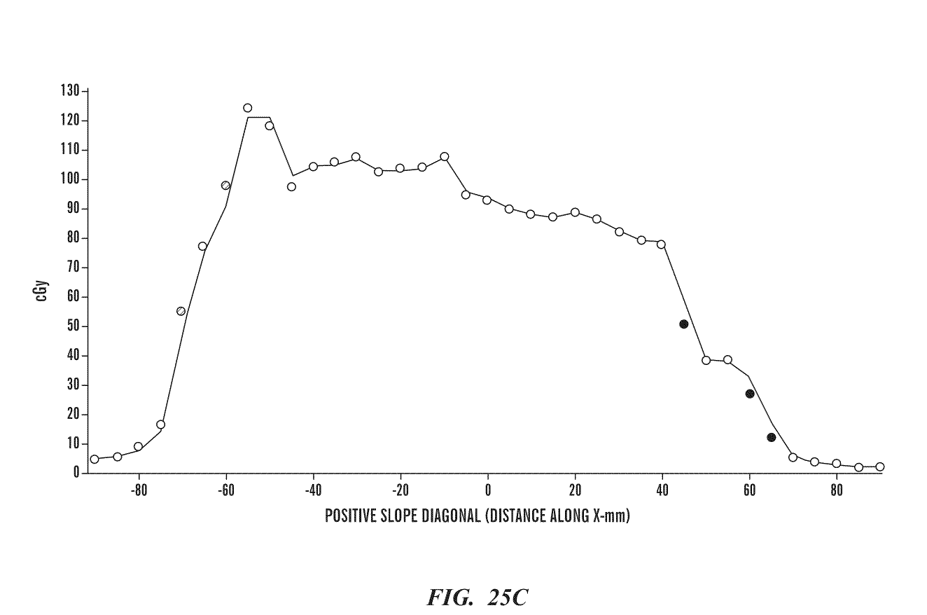

However, comparing all delivered plans with the computed plan from treatment planning system, using the 3 mm distance-to-agreement and a 3% dose difference, it was observed that all plans were within the 2% absolute difference. The passing rate of the reference plan comparing to the stationary one was 91.2% for stationary delivery, and the passing rate for the other two plans (tracking delivery) were 90.1% and 92.2%. It was observed that the absolute differences of both tracking plans comparing to the stationary plan was 1.2% and 1.09%. Comparing the stationary IMRT plan with the tracking plans, it was observed that the CAX doses were 92.34 cGy, and 93.48 cGy, respectively. The difference was −0.87%. The same effect of the difference in high gradient region was observed (FIG. 25).

FIG. 25 depicts comparison of the IMRT plan with the tracking one, where FIG. 25(a) depicts inplane profile, FIG. 25(b) depicts crossplane profile, and FIG. 25(c) depicts diagonal profile. Yellow dots denote the dose differences within 1%; blue and red dots denote dose differences higher and lower than 1% for the specific profile. In FIG. 25(a), Y-axis motion (mm) is provided along the x-axis and Dose (cGy) is provided along the y-axis. In FIG. 25(b), X-axis motion (mm) is provided along the x-axis and Dose (cGy) is provided along the y-axis. In FIG. 25(c), Positive Slope Diagonal (Distance along x-mm) is provided along the x-axis and Dose (cGy) is provided along the y-axis.

Analyzing the high gradient region, the maximum absolute recorded deviation from the reference plan was 1.9% for the 3D conformal plan, and 2.4% for the IMRT plan. It was observed that 32 of 445 diodes recorded the dose deviation outside the ±1% range for the IMRT plan, and only 4 diodes recorded the absolute deviation greater than 2%. However, this did not influence the passing rate of the plan compared to the passing rate of the planning system with the stationary plan. The passing rate for the former was 92.2% and that for the latter one was 91.7%. The absolute difference of both plans was 0.55%. The difference level for both the 3D conformal and IMRT plans was clinically acceptable. Summarized experimental results were presented in Table 4.

TABLE 4
Overview of the various experimental results. Ref denote the
reference plans, whereas T denote plans with tracking.

| Couch performance test | | | Mechanical tracking test | | |
|---|---|---|---|---|---|
| Accuracy [mm] | | | Accuracy [mm] | | |
| X | Y | Z | X | Y | Z |
| 0.10 | 0.10 | 0.12 | 0.12 | 0.14 | 0.18 |
| SD [mm] | | | Repeatability [mm] | | |
| X | Y | Z | X | Y | Z |
| 0.10 | 0.10 | 0.13 | ±0.2 | ±0.2 | ±0.2 |

Dosimetry tests

| 3D conformal plan; CAX dose [cGy] | | | IMRT plan; CAX dose [cGy] | |
|---|---|---|---|---|
| Ref3D | T1 | T2 | RefIMRT | TIMRT |
| 213.75 | 213.24 | 210.94 | 92.34 | 93.48 |
| — | +0.21% | +1.19% | — | −0.87% |
| Passing rate (10-3-3) [%] | | | Passing rate (10-3-3) [%] | |
| Ref3D | T1 | T2 | RefIMRT | TIMRT |
| 91.2 | 90.1 | 92.2 | 92.2 | 91.7 |

The supplemented studies (I. Buzurovic, M. Werner-Wasik, T. Biswas, J. Galvin, A. P. Dicker, Y. Yu, T. Podder, “Dosimetric Advantages of Active Tracking and Dynamic Delivery”, Med. Phys. 37, 3191 (2010); I. Buzurovic, K. Huang, M. Werner-Wasik, T. Biswas, A. P. Dicker, J. Galvin, Y. Yu, T. Podder, “Dosimetric Evaluation of Tumor Tracking in 4D Radiotherapy”, Int. J. Radiat. Oncol. Biol. Phys. 78, 5689 (2010)) included the evaluation of dosimetric advantages of tumor motion tracking and the irradiation of normal lung and spinal cord. Using the proposed active tracking technique it was found that the irradiated PTV was 20-30% less for average tumor motion of 1.5 cm, which suggested significant sparing of healthy tissue. While assessing the dose it was concluded that approximately 20% of the healthy lung received 4-8 Gy less when the tumor tracking technique was used.

The experimental results showed that the EPT without additional attachments or changes in its design and with the existing power and motors can perform real-time 4D tracking. The modification of the control systems can provide the tracking provisions. Since the existing motors and driving mechanisms were used, the proposed tracking methodology should not have any limitation in clinical implementation. The second set of experiments validated the system capabilities to follow desired trajectories, regardless of the slope and shape of the breathing trajectories. The third set of measurements verified that, with proper implementation, tracking methodology did not influence the plan quality and delivery. The critical issue for the clinical implementation might be the correlation between the tumor motion and table motion, i.e., choosing the right moment to start tracking. This problem can be solved using the position sensor which can sense the maximum extent of the inhale-exhale (inhalation and exhalation). Furthermore, it is possible to integrate the couch motion signal to linear accelerator beam control to turn off the beam, if the breathing trajectory is out of tracking limits.

In the following part, some of the previously reported data on tumor tracking accuracies were compared to the results of this study. The average root-mean-square differences between the measured data and modeled data for a robotic couch tracking were 0.02 and 0.11 cm for step changes of 1 and 3 cm, respectively (W. D. D'Souza, T. J. McAvoy, “An analysis of the treatment couch and control system dynamics for respiration-induced motion compensation”, Med. Phys. 33, 4701-4709 (2006)). The similar type of experiments revealed the EPT accuracy of 0.12 mm using the proposed approach. The reported systematic tracking errors were below 0.14 mm using a novel platform for the image guided stereotactic body radiotherapy. (T. Depuydt, D. Verellen, O. Haas, T. Gevaert, N. Linthout, M. Duchateau, K. Tournel, T. Reynders, K. Leysen, M. Hoogeman, G. Storme, M. D. Ridder, “Geometric accuracy of a novel gimbals based radiation therapy tumor tracking system”, Radiotherapy and Oncol. 98, 365-372 (2011).) The integration of the electromagnetic real-time tumor position monitoring into a MLC-based tracking system was investigated (A. Krauss, S, Nill, M. Tacke, U. Oelfke, “Electromagnetic real-time tumor position monitoring and dynamic multileaf collimator tracking using a Siemens 160 MLC: Geometric and dosimetric accuracy of an integrated system”, Int. J. Radiat. Oncol. Biol. Phys. 79, 579-587 (2011)), and the sub-millimeter tracking accuracy was observed for the two-dimensional target motion. The proposed EPT tracking errors were within the same range for the three-dimensional target motions. The investigation of the accuracy of the single kV-imager based DMLC tracking for static-gantry delivery revealed that the mean root-mean-square tracking error was 0.9 mm (perpendicular to MLC) and 1.1 mm (parallel to MLC) for the thoracic/abdominal tumor trajectories and 0.6 mm (perpendicular) and 0.5 mm (parallel) for the prostate trajectories (P. R. Poulsen, B. Cho, A. Sawant, D. Ruan, P. J. Keall, “Dynamic MLC tracking of moving targets with a single kV imager for 3D conformal and WIRT treatments”, Acta Oncol. 49, 1092-1100 (2010)). It can be noticed that the experimental results from this study were comparable to the already published results, no matter which specific tracking technique was used.

Based on the dosimetric studies (I. Buzurovic, Y. Yu, T. K. Podder, “Active Tracking and Dynamic Dose Delivery for Robotic Couch in Radiation Therapy”, Proc. of IEEE Int. Conf. on Eng. in Medicine and Biol., 2156-2159 (2011); I. Buzurovic, M. Werner-Wasik, T. Biswas, J. Galvin, A. P. Dicker, Y. Yu, T. Podder, “Dosimetric Advantages of Active Tracking and Dynamic Delivery”, Med. Phys. 37, 3191 (2010); I. Buzurovic, K. Huang, M. Werner-Wasik, T. Biswas, A. P. Dicker, J. Galvin, Y. Yu, T. Podder, “Dosimetric Evaluation of Tumor Tracking in 4D Radiotherapy”, Int. J. Radiat. Oncol. Biol. Phys. 78, 5689 (2010)) and presented tracking methodology, clinical implementation of real-time tracking can be employed for the patient treatment benefits.

In the present specification, we presented a novel method and experimental implementation of real-time tumor tracking. The experimental results of tumor tracking using the Elekta Precise Table were presented. The tumor tracking test was performed in all three dimensions, and the results confirmed the simulation results. The couch performance tests revealed the table motion accuracy of 0.10 mm, 0.10 mm, and 0.12 mm in X, Y and Z direction, respectively. The mechanical tracking tests resulted in the tracking accuracy within sub-millimeter levels (0.12 mm, 0.14 mm, and 0.18 mm for X, Y, and Z axes), with a motion repeatability of ±0.2 mm. The dosimetric tests with external radiation beam resulted in a maximum dose deviation of 1.19% at CAX, and 2.4% inside the high gradient dose region taking both the 3D conformal and IMRT plans into account.

The study revealed that real-time tumor tracking was feasible using the existing robotic tables with modifications in control systems. The table maintains its original functionality, and the additional equipment was added in an appropriate manner. The experimental results showed that the treatment couch could be successfully used for real-time tumor tracking. This tracking technique using treatment couch potentially offers a simple and effective method to minimize the irradiation on healthy tissues. The present invention can be adapted for both regular and irregular breathing patterns as well as different breathing periods. Moreover, the system evaluation can be performed using the beams delivered with variable gantry angles as in the volumetric modulated arc therapy.

### Part 9

### New Invention on Motion-Mitigated Imaging and Verification of Motion Compensation

At CT-simulation stage and/or initial radiation therapy delivery (or subsequent periodic verification) stage, the planned couch tracking motion can be carried out if the tracking couch of the present invention has been installed in said CT-simulation and/or radiation delivery suites. Upon such tracking motion, the moving tumor shall appear under imaging to be either stationary, moving marginally, or moving with reduced excursion, depending on the tracking strategy chosen (aggressive, moderate, or soft). This is an added confirmation of the tracking strategy being deployed for the patient.

The challenge of such imaging and verification is that parts of the anatomy of the living subject are stationary relative to the room coordinates while another part (the physiological motion of interest) is moving. By tracking the moving anatomy thus making it appear either stationary or moving with substantially reduced magnitude in the room coordinates using the programmable couch platform, which is a main aspect of the present invention, the anatomy which is previously stationary relative to the room coordinates then appears to be moving. Thus it is not possible to generate a complete stationary rendering of the whole anatomy of the living subject at any instant.

The enabling technology taught in the present invention involves applying medical imaging such as, for example, computed tomography, cone beam CT, tomosynthesis, ultrasonography, planar x-ray imaging or fluoroscopy, electromagnetic transponder signals, optical imaging, stereoscopic surface imaging, positron emission tomography, SPECT, a plurality of times, including at least one time with the tracking strategy switched on, and at least one time with the tracking strategy switched off. Image segmentation and co-registration using any standard methodology can then be applied to combine the stationary part of the anatomy from the image set without tracking, and the physiologically moving anatomy from the image set with tracking, to render a resulting motion-compensated image set. By examining this image set and comparing the extent of physiological motions such as in 4-dimensional CT or conebeam CT modalities, one can also verify if the chosen tracking strategy is effective in mitigating, reducing or eliminating apparent motions of a therapeutic target.

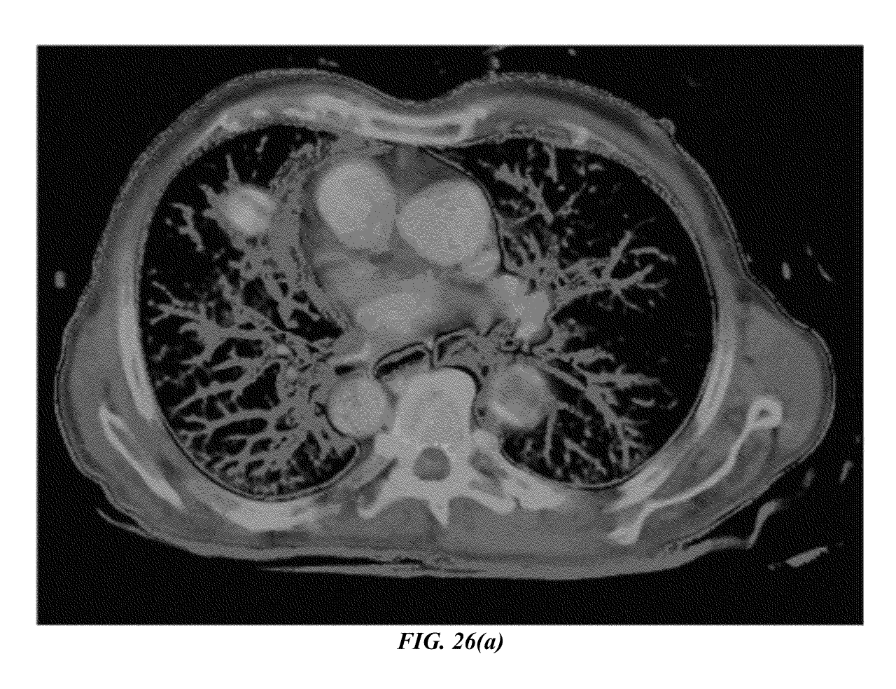

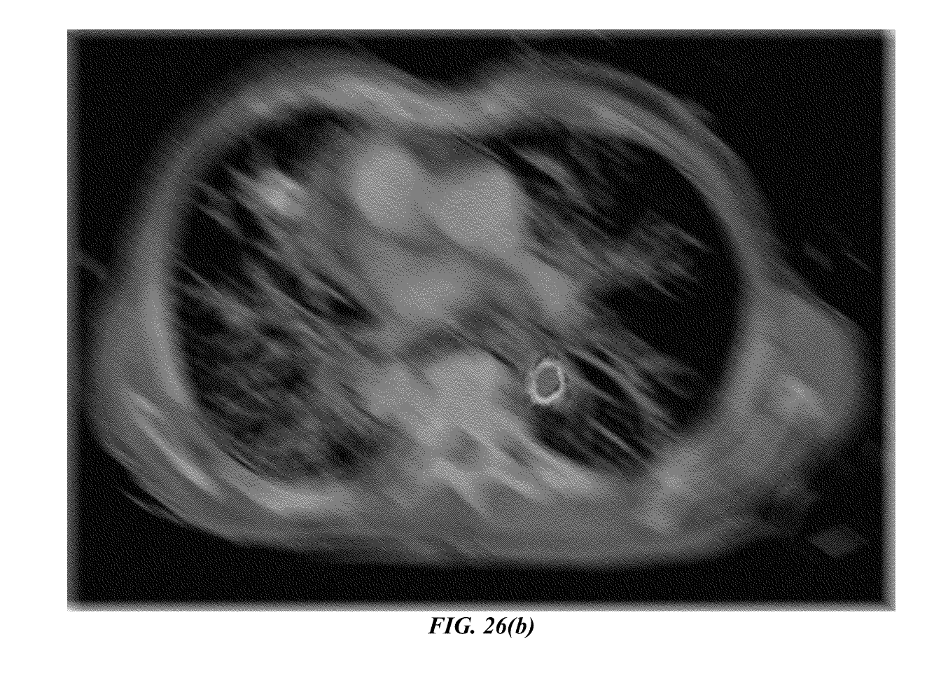

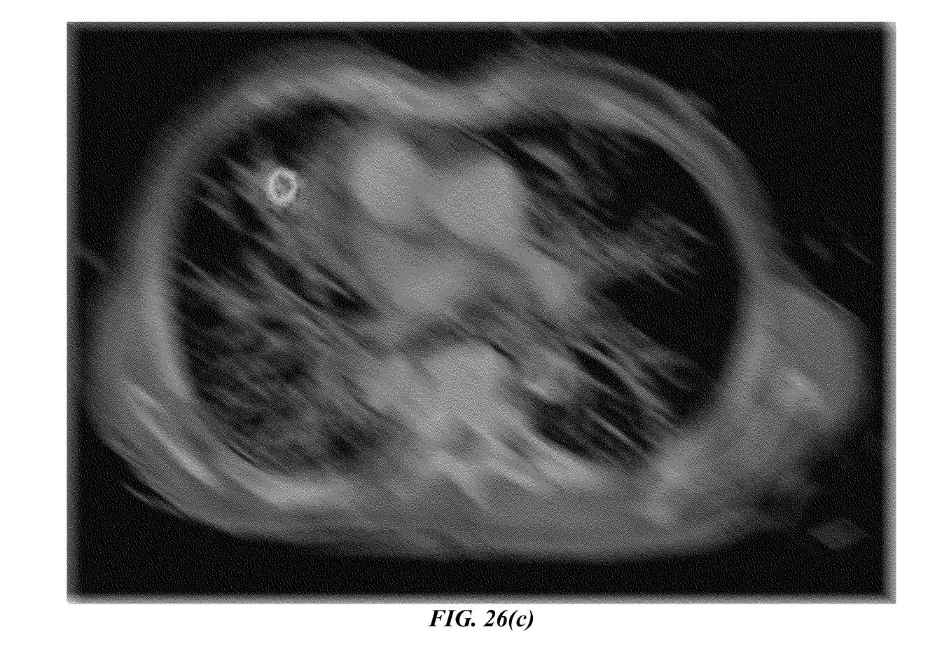

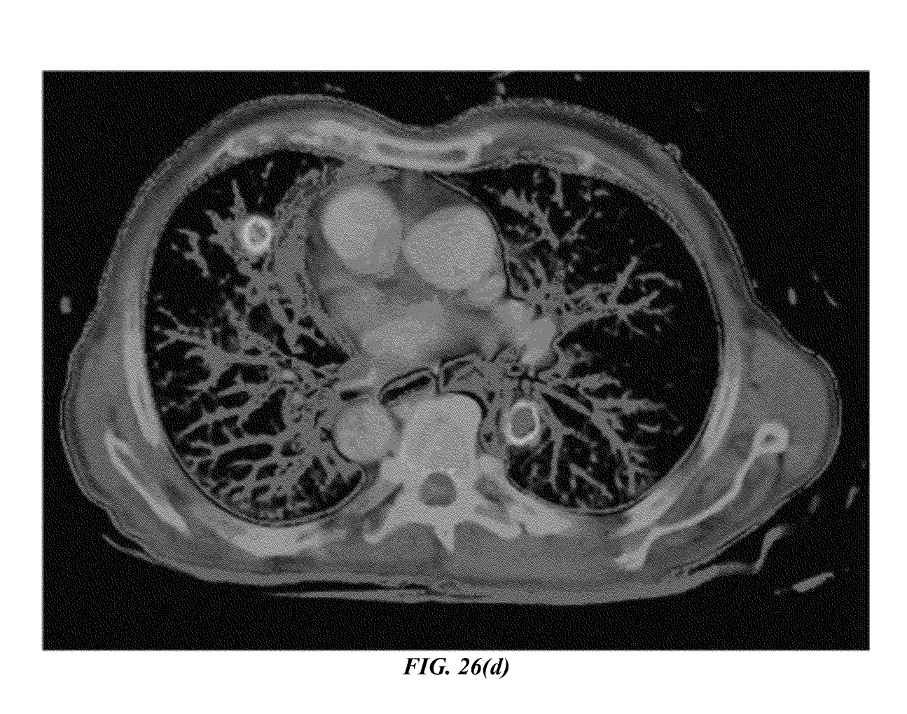

FIGS. 26(a)-(d) illustrate this novel methodology.

FIG. 26(a) depicts imaging lung tumors while the supporting couch is stationary. Two separate lung tumors in patient's posterior left (i.e., image right) and anterior right (i.e., image left) can be seen in 4-dimensional CT or conebeam CT to move due to respiratory motion. As a consequence the tumors appear to occupy a greater spatial extent of the lung volume in the room coordinate.

FIG. 26(b) depicts imaging the patient's posterior left lung tumor while the supporting couch is programmed to undergo counter-motion to this tumor's physiological motion. As a consequence, this tumor appears to be stationary in the room coordinate and more sharply defined, while the rest of the patient's anatomy displays motion blur.

FIG. 26(c) depicts imaging the patient's anterior right lung tumor while the supporting couch is programmed to undergo counter-motion to this tumor's physiological motion. As a consequence, this tumor appears to be stationary in the room coordinate and more sharply defined, while the rest of the patient's anatomy displays motion blur.

In FIG. 26(d), by segmenting and combining the non motion-blurred parts of FIGS. 26(a)-(c), a fully motion-compensated image set can be obtained. In this example, a total of 3 sets of imaging scans involving one stationary couch position and two different couch tracking motion patterns are needed to obtain this final image set.

### Part 10

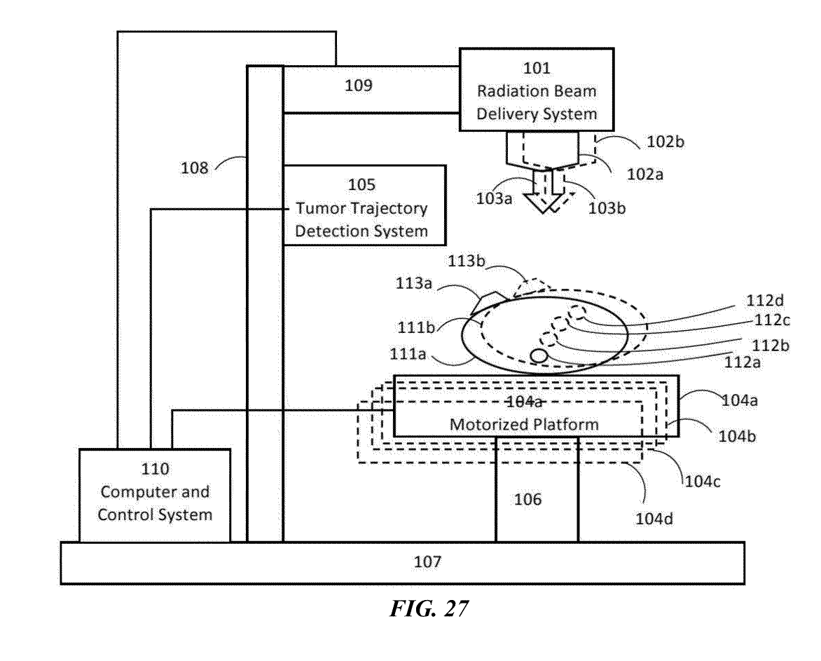

FIG. 27 depicts the following: 101—linear accelerator head that generates and assists in delivering radiation treatment beam; 102—beam shaping device that shapes the radiation bean based of dosimetric plan data and attenuate unwanted radiation; 103—shaped radiation beam; 104—motorized platform that supports the subject and moves to compensate tumor motion; 105—tumor trajectory detection system that find the tumor motion in real-time; 106—support mechanism for the motorized platform; 107—attachment to the ground or fixed structure for references; 108—supporting structure; 109—gantry of the linear accelerator; 110—computer system and the controller system; controller system is consisted of PID and adaptic controllers; also contains Ae_ANN and AE-nLMS algorithms; 111—cross-section of a subject which contains the tumor (112); 112—tumor that is to be tracked and treated with radiation; 112a original/non-moving location, 112d extreme location during excursion due to physiodynamic effects; 113—external fiducial/marker/

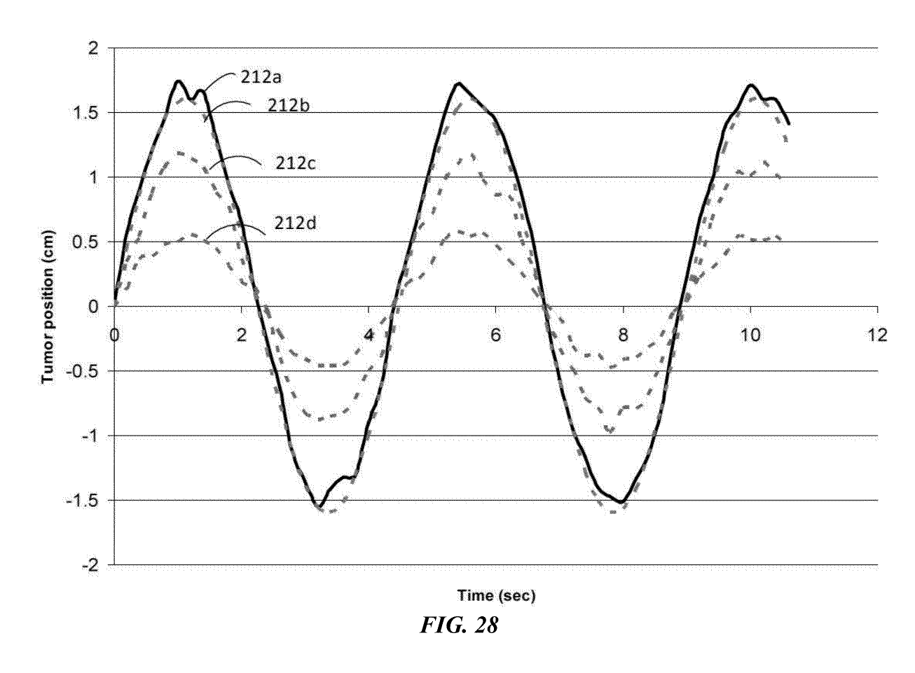

FIG. 28 depicts a tracking strategy, i.e., level of tracking of the tumor. In FIG. 28, Time (sec) is provided along the x-axis and Tumor position (cm) is provided along the y-axis. FIG. 28 depicts the following: 212a—original motion of the tumor; 121b—extreme level of tracking; 212c—moderate level of tracking; 212d—soft level of tracking.

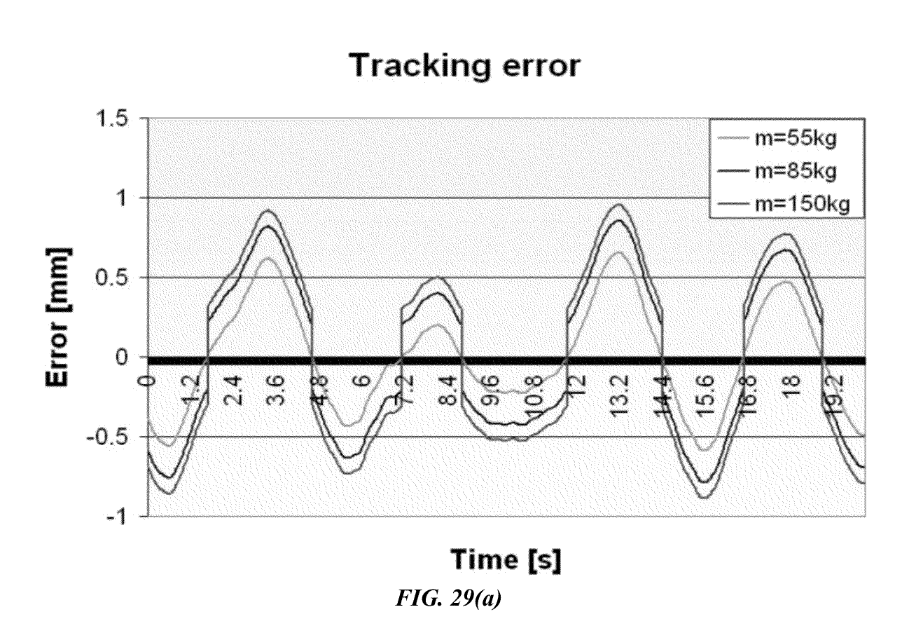

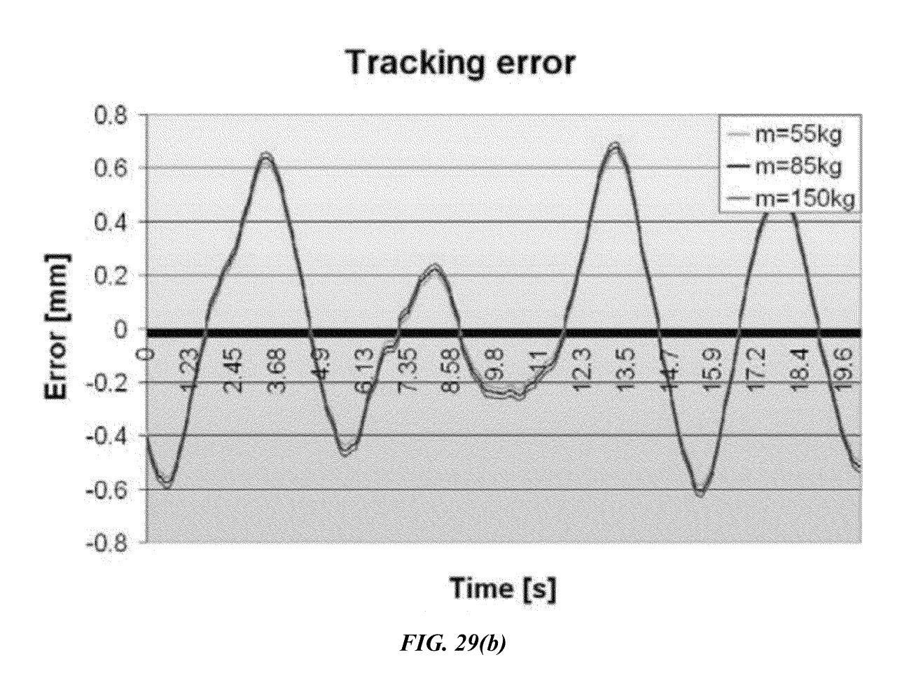

FIG. 29(a) depicts tumor tracking error for motorized platform when PID controller was used with different subject load. FIG. 29(b) depicts tumor tracking error for motorized platform when adaptive controller was uses with different subject loads. In FIG. 29, Time (s) is provided along the x-axis and Error (mm) is provided along the y-axis.

### Part 11

### Specification for Biofeedback, Training, Adaptive Tracking, 4D Simulation

Whereas patient undergoing treatment simulation and initial fractions of radiation therapy with couch tracking may be tense and overly reactive to moderate or more aggressive levels of tracking motion, the psychological/physiological aspects of such over-reaction can be effectively reduced by motion training, and more specifically, training with sensory feedback. For example, since the tracking motion is closely correlated to the patient's own respiratory motion, a pair of goggles can be used for the patient to visualize the motion curve as respiration/tracking takes place. This often has the positive effects that (a) the patient tends to self-control the amplitude of expiration so as to maintain a comfortable/tolerable level of tracking movements; (b) by feeling/visualizing as if one is controlling the couch movement via breathing, the patient tends to tolerate higher levels of tracking motion than without visual feedback. We term these types of motion trajectory feedback “biofeedback”, as it reinforces the notion that the couch motion is but a mechanical manifestation of one's own biological motion, and is therefore not something that needs further reacting to.

Other simple but effective sensory feedback techniques for regulating physiological motion and mitigating motion trajectory outliers include preprogrammed visual patterns or “light shows”, which can be delivered either through goggles or via light fixture panels in the simulator room and treatment room. The patient is prompted to synchronized breathing pattern with the sensory signal. Similarly, some patients may benefit from certain music chosen specifically for its rhythm pattern determined to be conducive to regulated tumor motion trajectory. Again, the optimal music may be determined at simulation and played during delivery.

Clearly, the purpose of biofeedback is to maximize the couch tracking magnitude without inducing counter-reaction from the patient. The optimal level of tracking under biofeedback can be determined through initial simulation training, in which the patient can self-experiment with different breathing levels and possibly feel empowered to optimize the tumor tracking strategy.

Of course, such training does not necessarily include biofeedback. In any case, the concept of 4D simulation as a method and as a device is heretofore introduced. As a method, this concept teaches using a programmable motion couch to determine the optimal level of tracking trajectory tolerable by the patient. This can be additionally aided by sensory-based biofeedback. The simulation can additionally involve 4D composite imaging, in which a plurality of images sets such as conebeam computed tomography (CBCT) are acquired, consisting of at least one set taken with couch in movement executing the chosen motion trajectory, and at least one set taken with couch stationary. Such image sets can be combined by extracting useful portions of the images, for example, where motion blurring is minimized. As a device, the concept teaches a new generation of radiotherapy simulators and/or simulation CT that should replace current simulators and CT's deployed in many radiation oncology departments. The new device is characterized by the addition of at least a programmable tracking couch, and more preferably of other sensory feedback aids described above. Whereas 3D simulation is a current standard step prior to radiation delivery, the new device permits true 4D simulation, which can be considered the new standard. It is to be noted that “4D simulation” is sometimes used as a term to describe current technique of acquiring CT or other images multiple times through a breathing cycle and tagging the image sets with the respiratory sensor such as the RPM or “belly bellow”. This latter technique does not involve moving the couch and is therefore not true 4D simulation under the presently described paradigm. Only a next generation 4D simulator facility has the full capability to offer 4D simulation for maximum patient safety, comfort and treatment quality, and reliability.

While several respiratory sensors are already in practical use and some image-based or electromagnetic motion sensors are available, the present invention also teaches the use of physiological sensors as additional “leading indicator” detectors of the onset of unplanned tumor motion. A number of physiological sensors exist in the market, such as skin conductance, ECG, EKG, respiration sensor, BVP as detailed below (Section D, “Data Collection”).

A preferred embodiment of the present invention includes using such physiological sensors as a device and method to detect and mitigate departures from planned motion trajectories.

A. Objective:

Cancer diagnosis and subsequent therapy can cause patients to experience considerable physiological, emotional/psychological stress, and anxiety. Such physiodynamic changes may cause in alteration of tumor motion due the change in respiratory and cardiac functions as well as undesired physical motion of the patient (especially for non-compliant patient) during radiation therapy, thereby compromising the accuracy of radiation treatment and patient safety. Physiodynamic signals (biofeedback) acquired during radiation therapy may be reliably correlated to breathing and cardiac motion patterns and internal tumor motions. Monitoring of physiologic events during therapy helps in capturing such changes, and assists in minimizing irradiation of normal lung tissues and critical organs. This improves the accuracy of radiation delivery as well as enhances patient safety.

B. Specific Aims:

The specific aims are as follows: (1) Find how the psychophysiological states affect the breathing and cardiac motion patterns during radiation therapy, (2) Assess the physiodynamic events that are detrimental to patient treatment and correlate these events to treatment uncertainty and patient safety, (3) Develop a mathematical model correlating lung tumor motion and the external fiducial motion and then to physiodynamic signals, (4) Determine a threshold for the physiodynamic events beyond which verification of the correlation between external fiducial and internal tumor motion may be required and accordingly updated the mathematical model, i.e., tumor trajectory, and (5) Develop prediction algorithms for harmful physiological events and develop a biofeedback-based closed-loop control system for improving treatment accuracy and also develop a viable action plan for patient's safety.

C. Research Strategy:

The main goal of this study is to measure the patient's physiological states for predicting and co-relating to patient's actions, physical movements (voluntary/involuntary), and to the motion of the tumor. To improve patient safety and enhance treatment quality, new technologies and techniques were developed and adopted. Since the survival of the patients has improved, it is important to minimize tumor margin to avoid unnecessary irradiation of normal lung tissue and adjacent critical structure for reducing long-term toxicities. Detection of physiodynamic events and implementing treatment corrections to account for them improves accuracy of dose delivery to the target volume while minimizing undesired/harmful dose to normal tissue or critical organs, as well as improving the patient safety. Moreover, a closed-loop automated technique of beam modulation can reduce the burden on the therapists. The present invention can be employed as part of a comprehensive strategy for fully dynamic robotic-assisted radiation therapy (RT), which incorporates patient physiological state prediction, and real-time active patient positioning.

Find how the psychophysiological states affect the breathing and cardiac motion patterns during radiation therapy.

Respiratory output is regulated by an automatic metabolic control system located in the brainstem and by a voluntary or behavioral control system in higher neural centers (von Euler, C., 1986. Brainstem mechanisms for generation and control of breathing pattern. In: Chemiack, N. S, Wid-dicombe, J. G., Eds., Handbook of Physiology, The Respiratory System, Control of Breathing, vol. 3, Part 1. American Physiology Society, Maryland, 1-67). A number of researchers have shown that sensory stimuli and mental activity alter breathing patterns (Mador. M., J. and Tobin, M. J., 1991. Effects of alterations in mental activity on the breathing pattern in healthy subjects. Amer. Rev. Respir. Dis. 144: 481-487; Boiten, F. A., 1993. Component analysis of task related respiratory patterns. Int. J. Psychophysiol. 15: 91-104). Emotions are linked to respiration (Masaoka, Y., and Homma, I., 1997. Anxiety and respiratory patterns: their relationship during mental stress and physical load. Int. J. Psychophysiol. 27: 153-159). Different emotional states show different breathing patterns (Boiten, F. A., Frijda, N. H., Wientjes, C. J. E., 1994. Emotions and respiratory patterns: review and critical analysis. Int. J. Psychophysiol. 17: 103-128). One of the conclusions of the meta-analysis of Cacioppo et al. (von Euler, id.) was that a better characterization of sympathetic and parasympathetic responses might provide some discriminative power to distinguish patterns of visceral activity associated with basic emotions. Moreover, the experience of several basic emotions has been consistently associated with changes in heart rate (Cacioppo, J. T., Berntson, G. G., Larsen, J. T., Poehlmann, K. M., Ito, T. A., 2000. The psychophysiology of emotion. In: Lewis, M., Haviland-Jones, J. M. (Eds.), The Handbook of Emotion. 2nd Edition. Guilford Press, New York, 173-191). Basic emotions are associated with distinct patterns of cardiorespiratory activity (Demaree, H. A., Robinson, J. L., Everhart, D. E. & Schmeichel, B. J., 2004. Resting RSA is associated with natural and self-regulated responses to emotional stimuli. Brain and Cognition, 56: 14-23; Demaree, H A., Schmeichel, B J., Robinson, J L., Piu, J., Everhart, D E, & Berntson, G. G., 2006. Up and down regulating facial disgust: Affective, vagal, sympathetic, and respiratory consequences. Negative emotional expressions: Behavioral, affective, and autonomonic consequences. Biological Psychology, 71: 90-99; Rainville, P., Bechara, A., Naqvi, N., and Damasio, A. R., 2006. Basic emotions are associated with distinct patterns of cardiorespiratory activity. Int. J. Psychophysiol. 61: 5-18). However, most of these studies (if not all) were performed under artificially created environments; none of them was conducted in radiation therapy environment. Patients in radiation therapy experience changes in emotional and psychological states, i.e., elevated anxiety, fear, and frustration, which potentially alter their breathing pattern and cardiac cycle. It is important to determine which psychological states influence the physiodynamics (breathing and cardiac pattern) as well as any other physical motions that are detrimental to radiation therapy. Signals captured by deploying the suites of biofeedback, i.e., physiodynamic sensors (EKG, BPV, skin conductance and temperature) would be analyzed for this purpose.

Milestone:

(1) Patient data collection, and (2) finding of psychophysiological states that affect the breathing and cardiac motion patterns.

Assess the physiodynamic events that are detrimental to patient treatment and correlate these events to treatment uncertainty and patient safety.

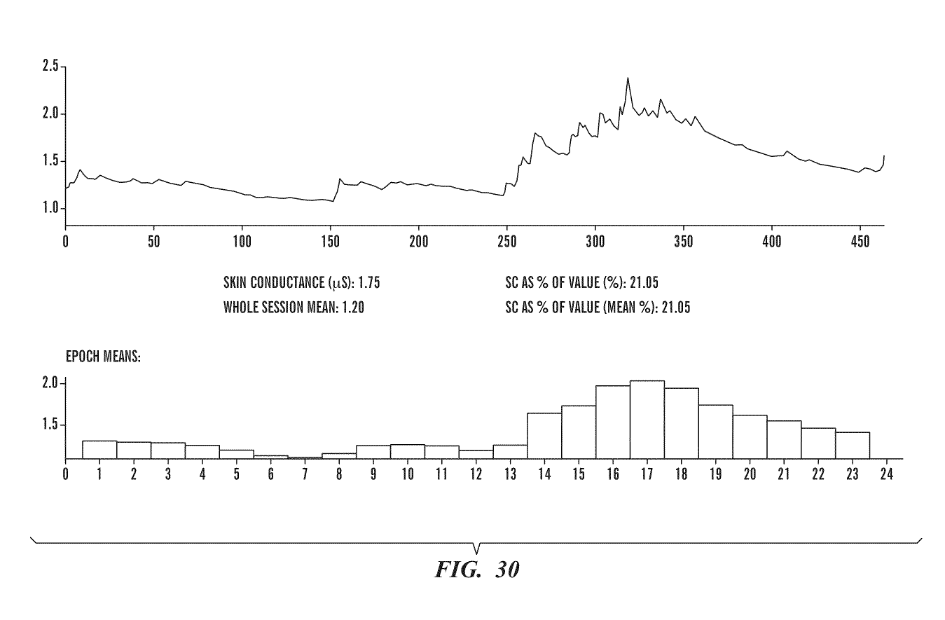

In this aim, the effects of the physiodynamic events on radiation dose delivered to tumor, normal tissues, critical structures, as well as patient safety are investigated. There exists evidence that the physiological activity associated with various affective states is differentiated and systematically organized. The physiological signals such as sympathetic power, parasympathetic power, mean inter-beat interval (IBI), mean BVP for cardiac activity; mean and slop of tonic activity level, mean, maximum and rate of phasic activity for electrodermal activity (FIGS. 30-33) are examined along with the parameters derived from each of the signals. These types of signals are selected because they can be measured non-invasively and are putting minimal burden to the patients. Additionally, electrodermal activity, various cardiovascular parameters, and jaw EMG are strong indicators of anxiety (Dawson, M. E., Schell, A. M., Filion, D. L., 1990. The electrodermal system. J. T. Cacioppo and L. G. Tassinary (eds.), Principles of psychophysiology: Physical, social, and inferential elements, Cambridge University Press, New York, 295-324; Lacey, J. L., Lacey, B. C., 1958. Verification and extension of the principle of autonomic response stereotypy,” American Journal of Psychology 71: 50-73; Smith, C. A., 1989. Dimensions of appraisal and physiological response in emotion. Journal of Personality and Social Psychology, 56: 339-353). In general, these indicators, comparing with baseline signals, can be correlated with anxiety such that higher physiological activity levels can be associated with greater anxiety (Smith, id.; Wright, R. A., Kirby, L. D., 2001. Effort determination of cardiovascular response: An integrative analysis with applications in social psychology. M. Zanna (ed.), Advances in experimental social psychology, Academic Press, 33: 255-307). First, these signals can be correlated with the physiodynamic events (as mentioned in Specific Aim 1 above), then the severity of the event can be assessed for impact of patient treatment deviation and patient safety.

Milestones:

(1) Detection of physiodynamic events from signals of physiological/biofeedback sensors, (2) establishment of the correlation between physiodynamic events and detrimental effects on patient treatment and safety.

Develop a mathematical model correlating lung tumor motion and the external fiducial motion and then to physiodynamic signals.

Since the lung tumor, (i.e., internal tumor) motion cannot be tracked continuously (note that electromagnetic beacon from Calypso cannot be used for lung cases), external fiducials are important surrogates for tumor motion. However, lung tumors encaged in thorax exhibit a variety of 3D motion trajectories which are different from the motion profiles of the fiducials placed externally on the body. Therefore, the success of the tumor tracking and thereby accuracy of radiation dose delivery to tumor significantly depend upon the accuracy of the correlation between the motion of the internal tumor and the motion of the external fiducials. We developed mathematical models for correlating tumor motion to external fiducial motion as well as to physiological signals. We introduced 6 dof electromagnetic (EM) sensor array (Aurora from NDI, Ontario, Canada) as external fiducial. These sensors can provide data at more than 100 Hz and submillimeter accuracy. Two sets of correlation models are developed. One set with tumor motion trajectory from 4D-CT data and another set using the tumor motion profile obtained from CyberKnife treatment. These models should (at least theoretically) match each other and they are used for radiation beam modulating in linac or guiding the robotic system. Our previous experience in this regard is helpful (Huang, K., Buzurovic, I., Yu, Y., Podder, T. K., 2010. A Comparative Study of a Novel AE-nLMS Filter and Two Traditional Filters in Predicting Respiration Induced Motion of the Tumor. IEEE Int. Conf. on Bioinformatics and Biomedical Engineering (BIBE), Philadelphia, Pa., 281-282; Buzurovic, I., Podder, T. K., Yu, Y., 2010. Prediction Control for Brachytherapy Robotic System. J. of Robotics, vol. 2010, Article ID 581840, 10 pages, doi:10.1155/2010/581840).

Our aim is to find how much of the tumor motion information is reflected in physiodynamic signals, and whether these signals can be used for the realtime updating of the mathematical models developed for tumor motion using surrogate external fiducials. Thereby, we can feasibly eliminate actions such as the frequent x-ray imaging in CyberKnife or tumor tracking with regular linac (with MLC and/or couch).

We develop a robust mathematical model by mappings between stress-relaxation as inferred from physiological features and the movement-deformation of the internal tumor. It resembles a classification problem where the attributes are the physiological features and the target function is the movement of the tumor. The Support Vector Machine (SVM), pioneered by Vapnik (Vapnik, V. N., 1998. Statistical Learning Theory. New York: Wiley-Interscience) is an excellent tool for classification problems (Burges, C. J. C., 1998. A tutorial on Support Vector Machines for pattern recognition. Data Mining and Knowledge Discovery 2: 121-167). Its appeal lies in its strong association with statistical learning theory as it approximates structural risk minimization principle. Good generalization performance can be achieved by maximizing the margin, where margin is defined as the sum of the distances of the hyperplane from the nearest data points of each of the two classes. It is observed that the support vector machine outperformed several other popular classification techniques when applied to a physiological pattern classification task involving human-machine interaction (Rani, P., Liu, C. C., Sarkar, N., Vanman, E., 2006. An empirical study of machine learning techniques for affect recognition in human-robot interaction. Pattern Analysis and Applications, 9: 58-69). As a result, we can adapt this method to build a model for tumor movement due to stress.

The Support Vector Machine is a linear machine working in a high dimensional feature space formed by an implicit embedding of low dimensional input data into a feature space through the use of a nonlinear mapping. This allows using linear algebra and geometry to separate the data that is normally separable only with nonlinear rules in the input space. The problem of finding a linear classifier for a given input data with known class labels can be described as finding a separating hyperplane in the feature space. Usually, to deal with the nonlinearly separable problems, a nonnegative slack variable generalizes the linear classifier with soft margin. To allow efficient computation of inner products directly in the feature space and circumvent the difficulty of specifying the non-linear mapping explicitly, all operations in learning and testing modes are done in SVM using so-called “kernel functions” satisfying Mercer conditions. Radial basis function (RBF) based kernel often delivers better performance and are applied to our task. The most distinctive fact about SVM is that the learning task is reduced to a dual quadratic programming problem by introducing the Lagrange multipliers. The corresponding Lagrange multipliers are non-zero only for the support vectors, those training points nearest to the hyperplane, which induces solution sparseness. The SVM approach is able to deal with overfitting by allowing for some misclassifications on the training set. This makes it particularly suitable for affect recognition because the physiological data could be noisy. Another important advantage of SVM is that the quadratic programming leads in all cases to the global minimum of the cost function. With the kernel representation, SVM provides an efficient technique that can tackle the difficult, high dimensional modeling problem.

We also use a Bayesian classification method that can be employed to predict the frustration level and also use adaptive neuro-fuzzy techniques for emotion detection, which can be important precursors of potential patient movements or tumor motions. With advancement of the technology, it is now feasible to inference the tumor position from surrogate breathing motion signal from external markers (Ahn, S., Yi B., Suh Y., et al., 2004. A feasibility study on the prediction of tumor location in the lung from skin motion, Br J Radiol 77: 588-596; Hoisak, J. D., Sixe K. I, Tirona R., et al., 2004. Correlation of lung tumor motion with external surrogate indicators of respiration. Int J Radiat Oncol Biol Phys 60:1298-1306; Tsunashima, Y., Sakae T, Shioyama Y., et al., 2004. Correlation between the respiratory waveform measured using a respiratory sensor and 3D tumor motion in gated radiotherapy. Int J Rad One Biol Phys 60: 951-958; Schweikard, A., Glosser, G., Bodduluri, M., Murphy, M. J., Adler, A. R., 2000. Robotic motion compensation for respiratory movement during radiosurgery. Comput Aided Surg 5:263-277). However, in this approach two main issues are presently addressed: (1) the 3D model strongly correlates the internal tumor motion to the external signal, and (2) time delay in computing the location of the marker and correspondingly the tumor location in task-space are minimized. The external breathing signal can be measured using infrared cameras and markers. Recent studies showed a significant improvement of the adaptive capabilities of respiratory motion prediction filtering (Murphy, M. J., Jalden, J. Isaksson, M., 2002. Adaptive Filtering To Predict Lung Tumor Breathing Motion during Image-Guided Radiation Therapy. Computer-Assisted Radiology and Surgery (CARS), Heidelberg: Springer-Verlag, 539-544; Sharp, G. C., Jiang, S. B., Shimizu, S., Shirato, H., 2004. Prediction of respiratory tumour motion for real-time image-guided radiotherapy. Phys Med Biol 49:425-440; Vedam, S. S. Keall, P. J., Docef, A., Todor, D. A., Kini, V. R., Mohan, R., 2004. Predicting respiratory motion for four-dimensional radiotherapy. In the J. Med Phys 31: 2274-2283) using artificial neural network (Vedam, S. S., Murphy, M., Docef, A., George, R., Keall, P. J., 2005. Long-term prediction of respiratory motion with artificial neural network based adaptive filtering techniques. Medical Physics, 32: 1925). We can also use other predictive filters such as Kalman Filter (KF) and Extended Kalman Filter (EKF) (Yan, K., Podder, T. K., Yu, Y., et al., 2006. Online Parameter Estimation for Surgical Needle Steering Model using Extended Kalman Filter. Int. Conf. on Medical Image Computing and Computer Assisted Intervention (MICCAI), Copenhagen, Denmark, 321-329).

Milestone:

(1) Mathematical model for correlating tumor motion to external surrogate/fiducial motion, (2) mathematical model to correlated psychophysiological signals to tumor motion, and (3) mathematical model to correlated psychophysiological signals to external fiducial motion.

Determine a threshold for the physiodynamic events beyond which verification of the correlation between external fiducial and internal tumor motion may be required and accordingly update the mathematical model, i.e., tumor trajectory.

The magnitude and frequency of tumor in lungs depend on breathing pattern and cardiac cycle. The breathing pattern and cardiac cycle are strongly correlated with the psychological states of the patient. Radiation dosimetric studies provide us the information regarding the tolerable limits of tumor motion, i.e., the range of motion of tumor which does not alter the radiation dose distribution to the clinical target volume (CTV) significantly. The patient-specific threshold of the physiodynamic events are determined based on the tolerable range of motion of the tumor. Our experience in dosimetric study with 4D-CT data for acceptable limit determination are helpful for this project. (I. Buzurovic, T. K. Podder, Y. Yu, “Effects of Tumor Tracking Errors to the Quality of Radiation Therapy,” Int. J. Radiat. Oncol. Biol. Phys. 84(3) (supplement), S716-717, 2012; I. Buzurovic, K. Huang, M. Werner-Wasik, T. Biswas, A. P. Dicker, J. Galvin, Y. Yu, and T. Podder, “Dosimetric evaluation of tumor tracking in 4D radiotherapy,” Int. J. Radiat. Oncol. Biol. Phys. 78(3), S689-S689, 2010.) If the threshold is crossed, a set of new data of the tumor motion are acquired for updating the mathematical model of the tumor motion.

Milestone:

(1) Determination of patient-specific threshold, and (2) strategy for updating mathematical model.

Develop prediction algorithms for harmful physiological events and develop a biofeedback-based closed-loop control system for improving treatment accuracy and also develop a viable action plan for patient's safety.

Under this aim, we developed mathematical models which predict physiodynamic events such as excessive anxiety, stress or relaxation which can cause abnormal breathing, cardiac motion or physical motion of the patient that are potentially detrimental to treatment accuracy and patient safety. We use a Bayesian classification method that can be employed to predict the frustration level and also use adaptive neuro-fuzzy techniques for emotion detection, which can be important precursors of potential patient movements or tumor motions.

Numerous signals such as electrodermal activity, inter-beat interval (IBI), blood volume pulse (BVP), heart rate variability (HRV) frequency ratio, sympathetic and parasympathetic power trends, etc., gleaned from physiological sensor suite can provide us with a vast wealth of information. This information leads to predict the events that are detrimental to treatment accuracy and patient safety. Based on the severity index of the events, a well designed closed-loop adaptive controller can be deployed to manipulate the radiation beam.

Developed predictive algorithms are able to detect the detrimental events and assess the effect in advance so that control instructions are transmitted to the appropriate device (linac, patient positioning couch) or personnel (therapist, clinician, patient) to stop or minimize the harmful action occurrence.

Milestone:

(1) Physiodynamic events prediction algorithms, (2) testing and validation results of the efficacy and robustness of algorithms, (3) a biofeedback-based closed-loop controller, (4) simulation and testing results of the controller, (5) preclinical evaluation results of the predictive algorithms, the control strategy, and the action plan for patient safety.

Success of the proposed methodology enables tumor tracking with tighter margins using non-invasive, non-ionizing radiation. Implementation of the proposed technique for regular linacs brings improved, accurate and safer radiation treatment options to a broader patient-base in community hospital settings. The present technique provides a potential paradigm shift in radiation treatment.

FIG. 30 depicts sessions with 1 channel of skin conductance. The screen shows a line graph of the raw signal (top panel) and a trend graph of epoch means (bottom panel).

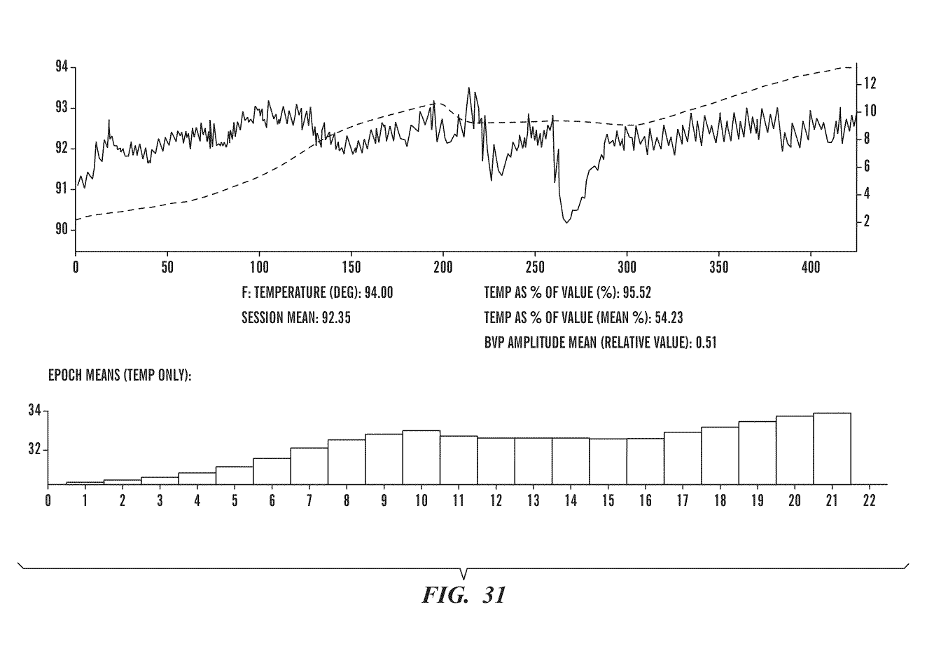

FIG. 31 depicts multi-modality sessions with BVP (amplitude) and Temp. The top panel shows the signal graphs while the bottom panel plots epoch means for the temperature channel.

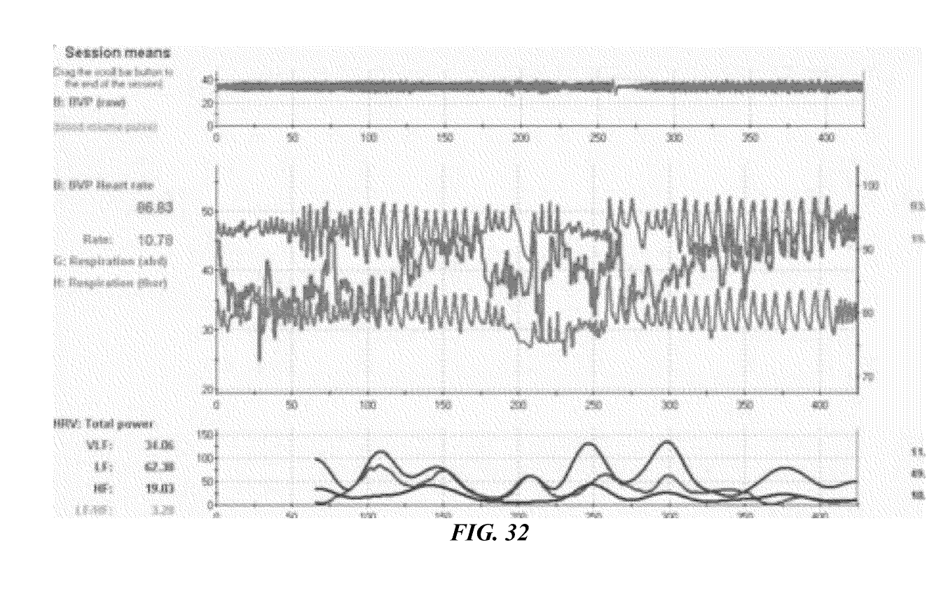

FIG. 32 depicts line graphs of the raw BVP or EKG signal and of the abdominal and thoracic respiration. Bottom panel with a line graph shows the total power output for each HRV band, VLF, LF and HF.

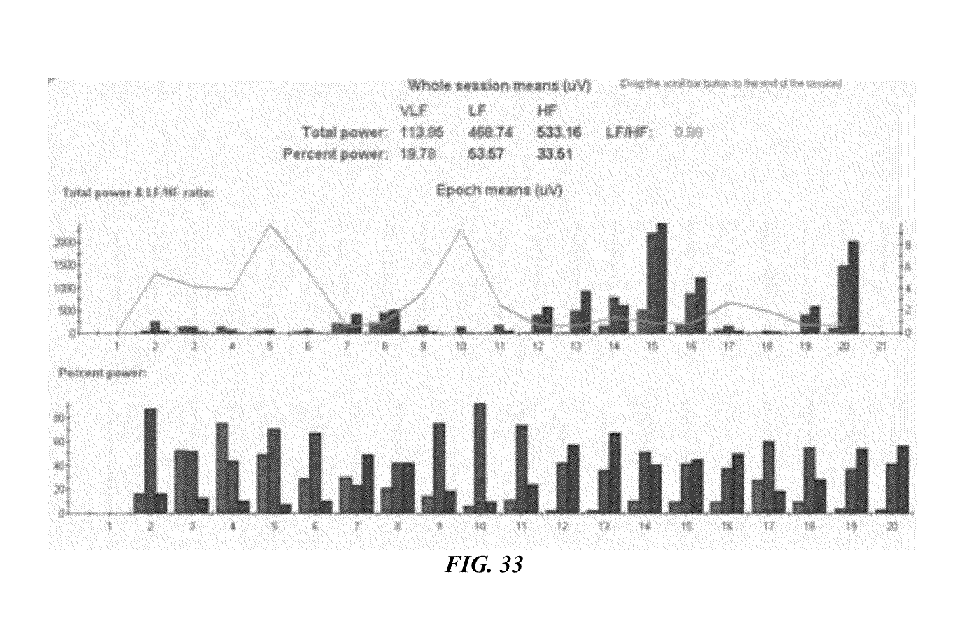

FIG. 33 depicts trend graphs of the total and percent power for the three standard HRV frequency bands, VLF, LF and HF. The LF/HF ratio is shown as a line on the top graph.

D. Data Collection & Analysis

Sample Size:

We collected 5 physiological sensing data, 2 external body motion (EBM) data and 2 internal tumor motion (ITM) data from each of the patients. Each of the 9 parameters have a 25% coefficient of variation in the same specimen and a 40% coefficient of variation among different patients. To learn the mean value with a 10% standard error of the mean (SEM) for the 9 parameters requires 40 subjects. 45 subjects were requested to cover potential incomplete experimental data collection.

Data Collection:

Data was collected from 45 patients, who had radiation therapy with CyberKnife, using the physiological non-invasive sensors attaching externally to the patient's body considering patient's comfort and radiation fields. In the Radiation Oncology Department at East Carolina University, 120-130 are treated annually patients with CyberKnife. About 60% of these patients are lung cancer patients. Therefore, recruitment of 45 patients was completed within 9 months.

Additional/new equipments included: a physiological sensor suite, a data acquisition module, a data analyzing module and laptop computer. The physiological sensor suite comprises a skin conductance sensor, a respiratory sensor, a temperature sensor, a heart rate variability (HRV) (or blood volume pulse (BVP)) sensor, electrocardiography (EKG) sensor, and an eletromyography (EMG) sensor.

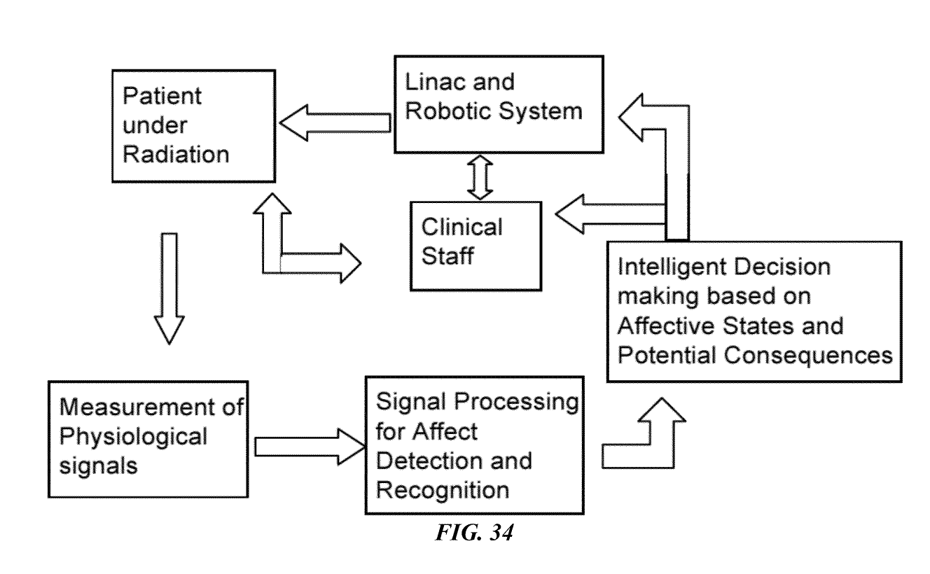

Data was collected from patients using the physiological non-invasive sensor attaching externally to the patient's body considering patient's comfort and radiation fields. These sensors do not need to be within the radiation field. Data was stored in a computer connected to the sensor for processing and analysis. The acquired signals were analyzed by using the proven commercial software as well applying presently developed algorithms. Subsequently, a close-loop controller based on physiological feedback for modulating radiation beam was developed and tested. A flowchart of the methodology is depicted in FIG. 34. A robust methodology was tested with a new set of patients.

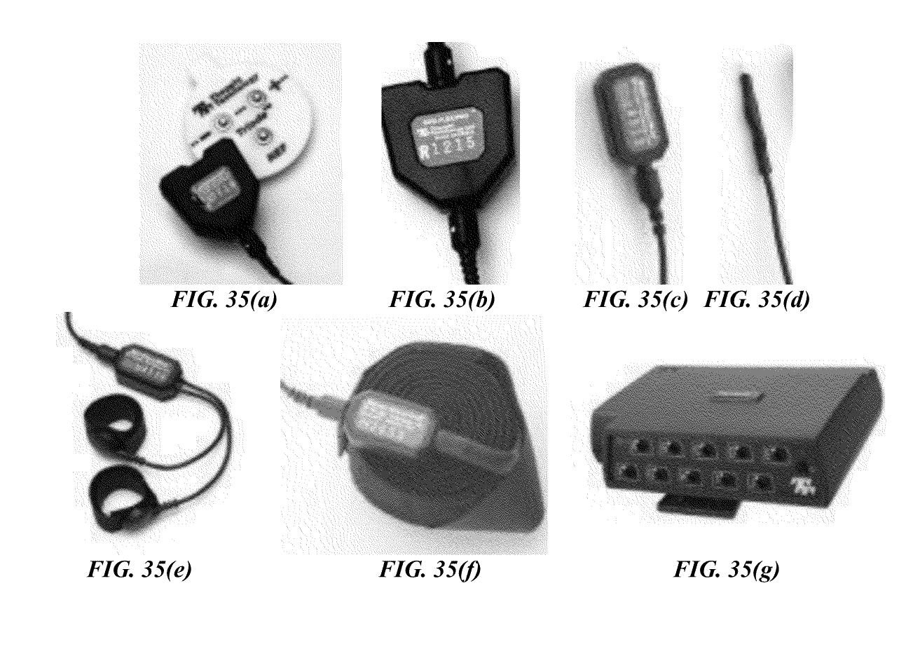

The required equipments were: a physiological sensor suite, a data acquisition module and a data analyzing module. The physiological sensor suite comprises a skin conductance sensor, a respiratory sensor, a temperature sensor, a heart rate variability (HRV) (or blood volume pulse (BVP)) sensor, electrocardiograph (EKG) sensor, and an eletromyography (EMG) sensor (FIG. 32). The Flexcomp-Infiniti™ (Thought Technology Ltd., Montreal, Canada) with commercially available sensors is an excellent data acquisition and physiological monitoring device for clinical and research applications. It offers 10 high-speed channels (2048 samples/sec.) with 14 bits of resolution (1 part in 16364) and can acquire data from any Thought Technology sensors (as listed in FIG. 35). These modules are widely used in various clinics and research institutes.

FIG. 35 depicts a physiological sensor suite and data acquisition equipment, where FIG. 35(a) depicts an EMG Sensor, FIG. 35(b) depicts an EKG Sensor, FIG. 35(c) depicts a BVP Sensor, FIG. 35(d) depicts a Temp. Sensor, FIG. 35(e) depicts a Skin Conductance Sensor, FIG. 35(f) depicts a Respiration Sensor, and FIG. 35(g) depicts a Flexcomp Infiniti (data acquisition module).

Surface EMG sensor: A pre-amplified surface electromyography sensor used with the ProComp Infiniti channels for RMS sEMG. It features a range switch in the sensor head to change filter settings ranging 0-400 μV for narrow-filter and 0-1600 μV wide-filter. It is compatible with Triode electrodes or extender cables for wider placement of electrodes. It is used for studying relaxation, stress, awareness of head, neck and lower back muscle tension or to stress/relaxation of specific muscle groups.

EKG sensor: Also a pre-amplified electrocardiograph sensor, for directly measuring heart electrical activity. This provides information about status of electrical activity of the heart as the emotion and psychological states of the patient change.

Skin Conductance sensor: To measure the conductance across the skin, normally connected to the fingers or toes. It is used for studying stress responses and basic self-regulatory responses. Skin conductance can vary from patient to patients in the range of 2-20 microsiemens. However, for a particular patient we need to find the baseline before radiation therapy, so that it can be subtracted to find the event triggered body/tumor motion.

Temperature sensor: This skin temperature sensor can measure temperature in the range from 10° C. to 45° C. (50° F.-115° F.). It is used for monitoring temperature biofeedback and increasing peripheral temperature as well as unconscious stress responses.

Respiration sensor: It is an easy fitting high durability latex rubber band fixed with velcro respiration belt for monitoring respiration rate from a patient. It can be worn either over thoracic region or over the abdominal region (even over clothing). Two channels of respiration are used for abdominal and (optionally) thoracic breathing pattern monitoring which can be correlated to internal organ/tumor motion (co-relation can be mathematically modeled using 4D-CT). Slow deep abdominal breathing helps with relaxation and can be used for lowering the heart rate. However, deep breathing can cause excessive movement of the tumor in the thoracic region.

HRV sensor: Heart rate variability (HRV) can be used for monitoring respiration and heart rate, using a blood volume pulse (BVP) sensor or an EKG sensor. They are useful for respiratory sinus arrhythmia (RSA) or to expand the adaptive range of the cardiovascular system (by increasing variability). Pulse detection (i.e., BVP) sensor housed in a small finger worn package are used to measure pulse rate of the patient.

We measure two types of motions that can cause significant errors in radiation therapy and can pose threat to patient safety. They are:

External body (or body parts) motion (EBM) of patient due to changes in physiological/emotional states, and

Internal tumor or target motion (ITM) due to changes of physiological/emotional states.

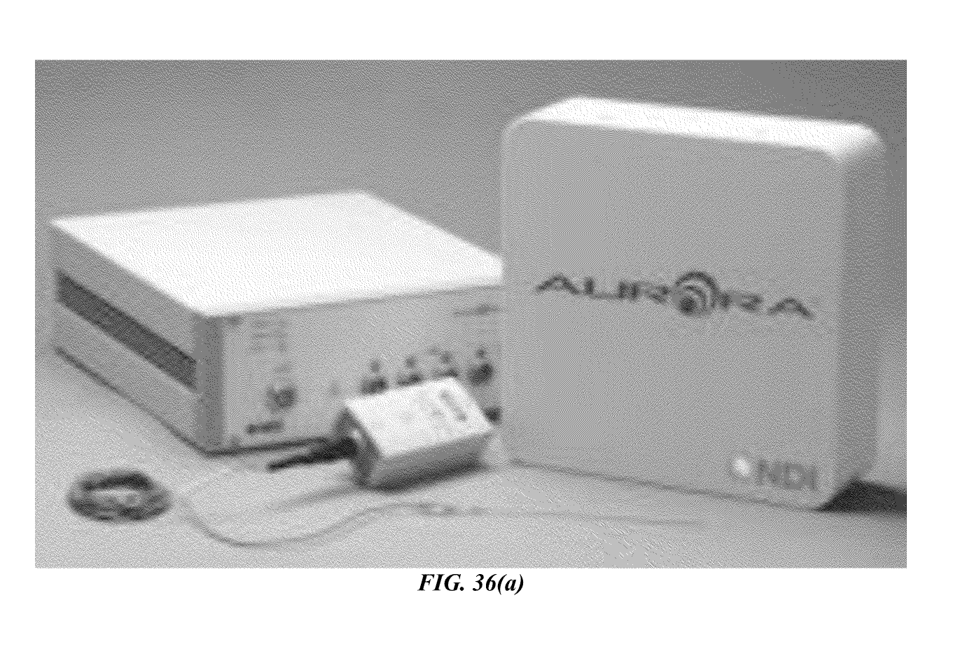

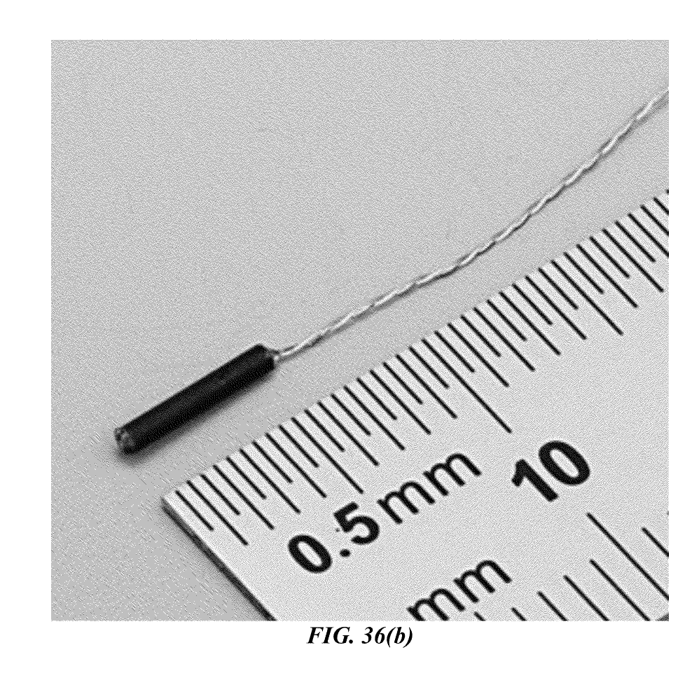

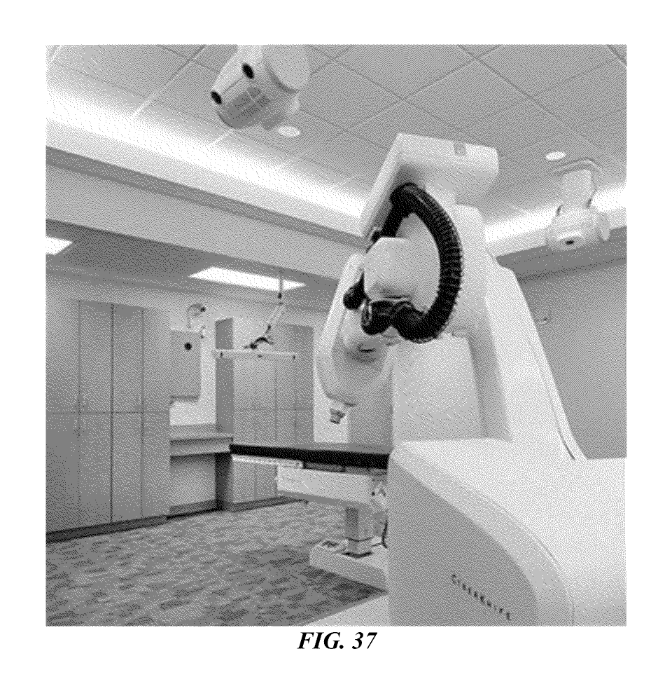

The EBM is measured using Aurora® electromagnetic sensor (Northern Digital Inc., Waterloo, Ontario, Canada), and high resolution optical tracking system fitted with CyberKnife robotic system (Polaris, NDI, Waterloo, Ontario, Canada) (FIGS. 36(a)-(b) and FIG. 37).

The ITM is measured or quantified by using 4D-CT (LightSpeed RT, GE Healthcare) acquired during planning CT imaging and also may be using 4D-CBCT (Elekta, Crawley, UK) acquired during radiation therapy.

Then we developed a mathematical model for determining strong correlation between the EBM and ITM for predicting the spatial and temporal location of the internal tumor from the trajectory of the external surrogate (or continuously measured data). This is very important for tracking the tumor for continuous delivery of radiation dose to the tumor precisely and safely, sparing normal tissue and critical organs. We developed a mathematical model to correlate these two motions, i.e., the EBM and the ITM, to the physiological data collected through the physiological event sensing system (more details are provided in SA3 section before).

FIG. 37 depicts a CyberKnife robotic system for radiation treatment.

FIG. 36 depicts motion capturing systems (Aurora EM sensors). FIG. 36(a) depicts Aurora EM Sensor package, and FIG. 36(b) depicts Aurora EM sensor (0.9 mm×6 mm).

Patient Selection:

Only the lung cancer patients for radiation treatment with CyberKnife treatment were considered for this study. Patient informed consent was taken on Institutional Review Board (IRB) approved consent form and IRB approved protocol was followed. This protocol was written following the guidelines suggested by CDMRP and related organizations. Patients with pacemaker and/or any other kind of electromagnetic support devices/systems were not included in the study.

On Treatment & Off Treatment Data:

The physiological, EBM and ITM data were collected during planning CT imaging, which is considered off treatment data. We also collected the said data during the actual radiation treatment of the patient (i.e., with radiation beam is on during CyberKnife treatment).

Statistical Analysis:

The physiological, EBM and ITM data were measured and recorded for statistical analysis. We used mean and standard deviation along with ANOVA/MANOVA to evaluate the data. The ANOVA/MANOVA were used to investigate whether there are significant main effects of the independent variables (i.e. physiological states) and whether there are significant interaction effects between independent variables in the data sets. To test specificity, we used post-hoc comparisons (such as Scheffe's and Tukey's) to find out where the differences were—which groups are significantly different from each other and which are not. The signal from any particular sensor may considerably vary from one patient to another. Thus, for each patient, we required to subtract the baseline signal (i.e., prestimulas—before radiation therapy) from the poststimulus (during radiation therapy and after) and define a deviation (standard deviation, variance, rate of change) that is a “sufficient” change (detection measure) for our applications.

Additional background information is disclosed as follows: Centers for Disease Control and Prevention: http://www.cdc.gov/cancer/lung/, website accessed in June 2011; Biswas, T., Hudson, S., Podder, T. K., Brinson, M., Efird, J. T., 2011. Demography and survival of lung cancer patient tobacco predominant Eastern North Carolina—a single institute study. Int J Radiat Oncol Biol Phys 81: 5582; McGarry R, Papiez L, Williams M, et al., 2005. Stereotectic body radiation therapy of early-stage non-small-cell lung carcinoma: phase I study. Int J Radiat Oncol Biol Phys 63: 1010-1015.

### Part 12

### Effects of Tumor Tracking Errors to the Quality of Radiation Treatment

Purpose

During radiation therapy, total compensation of thoracic tumor's motion may not be possible due to errors in tracking and prediction techniques. In this study, the dosimetric effects of the residual errors were investigated. Also, the error tolerance level, which would guarantee sufficient quality of the treatment plans, was determined.

Methods and Materials

The study was performed on 25 patients diagnosed with lung cancer. Eleven plans were generated for each patient, consisting of one clinically accepted initial plan and ten plans with induced tumor tracking errors, using CMS-XIO planning system.

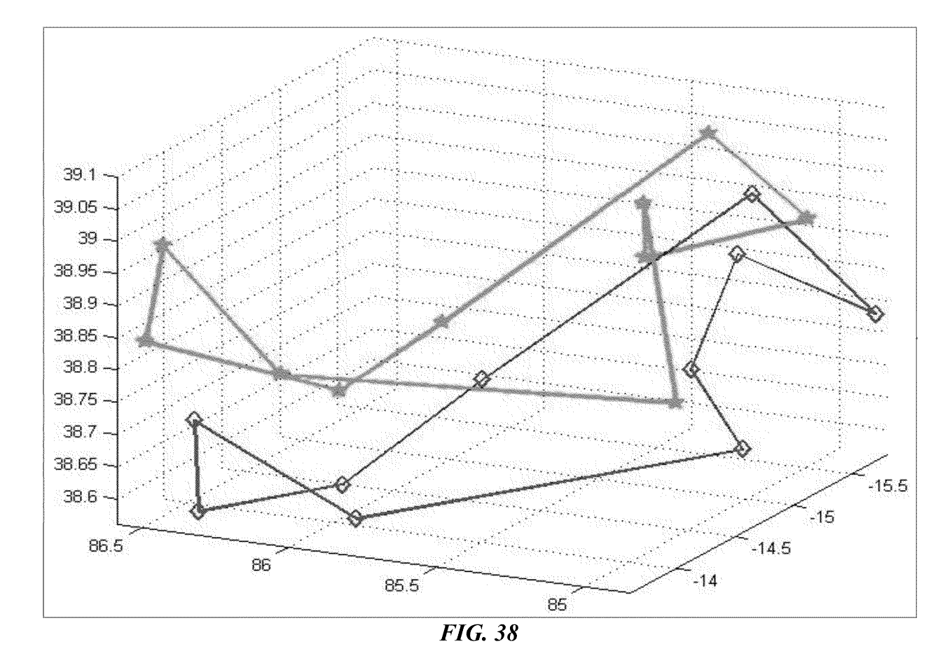

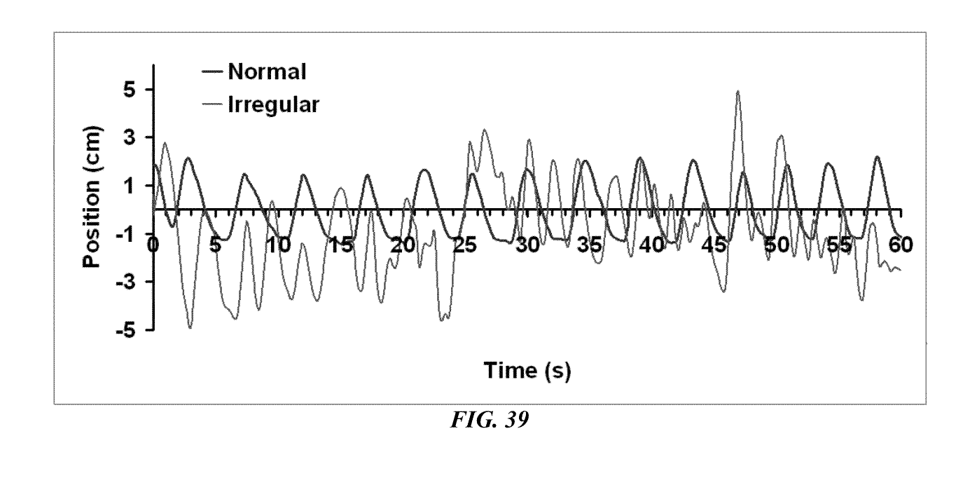

The initial plan was used for patient treatments. The other ten plans were generated by shifting the isocenter of the clinical plan to simulate tumor tracking errors from 1 mm up to 10 mm, as in FIG. 38. Tissue heterogeneity was corrected in all cases. The range of the tumor motion was within 2 cm for normal respiration in each direction, FIG. 39, and the respiration cycle was 3.5-7.3 s. In FIG. 39, Time (s) is provided along the x-axis and Position (cm) is provided along the y-axis.

Plans were compared considering dosimetric parameters including coverage of PTV (D99, D95, D50), volumes of normal lung receiving 5 Gy, 13 Gy, 20 Gy, 30 Gy dose (V5, V13, V20, V30) and D5 of the spinal cord. The initial plans were prescribed to D95 for patient treatments. For the purpose of this study, if the difference in D95 between the initial plan and the plans with induced error was more than 1%, it was considered unacceptable.

FIG. 38 depicts normal and irregular respiration signals (representative cases).

Results

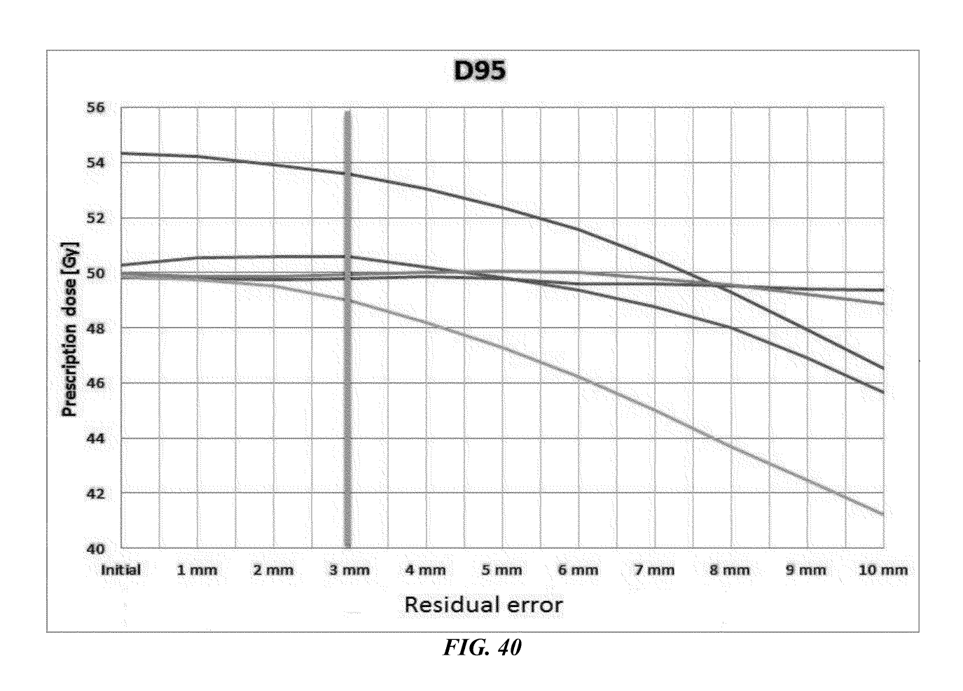

It was observed that D95 for 3 mm tracking errors was within a range of −1.09% to +1.98%. Tracking error limit of 3 mm still generated acceptable plans, FIG. 40. For the same error limit, the study showed that the average differences in the D99 of the PTV and CTV were within a range of 1.37% and 0.21%, respectively. In FIG. 40, Residual error (mm) is provided along the x-axis and Prescription dose (Gy) is provided along the y-axis.

Even in the extreme case (the respiration cycle is only 3.5 s, and the amplitudes of tumor motion in the X, Y, and Z directions were close to 2 cm), the difference in the D99 of the CTV was 0.9%. In all other cases, the differences were less than 0.71%.

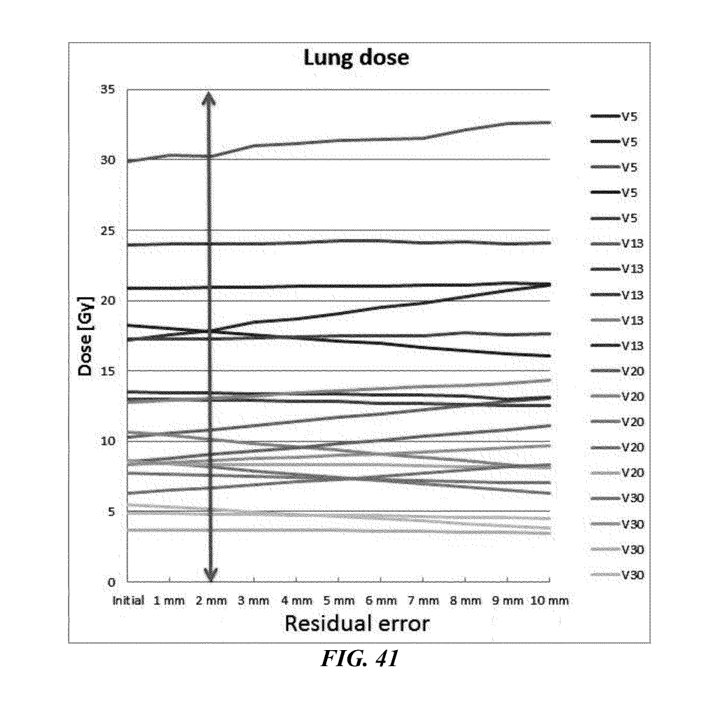

This study also revealed that the deviation in the delivered dose caused by the tracking error of 2 mm was insignificant for most of the anatomical structures. Dependency of residual errors to the lung doses was presented in FIG. 41. For example, in case of spinal cord, the average change in the V20 was 0.04%, while the average changes in the D5 were within 0.34 Gy. In FIG. 41, Residual error (mm) is provided along the x-axis and Prescription dose (Gy) is provided along the y-axis.

Based on these results, it would be reasonable to conclude that even when the overall error during tracking was 3 mm, 89% of the plans were still acceptable. With 2 mm errors, all the plans for all patients (100%) were acceptable. The dosimetric effects of random tracking errors in a range up to 3 mm were negligible.

### CONCLUSIONS

It can be concluded that during tracking it is not necessary to track respiratory peaks (which appear for short periods of time), and the tumor tracking trajectories can be smoothed.

Therefore, the high frequencies of tumor motion can be excluded during real-time tumor tracking.

Although some of various drawings illustrate a number of logical stages in a particular order, stages which are not order dependent can be reordered and other stages can be combined or broken out. Alternative orderings and groupings, whether described above or not, can be appropriate or obvious to those of ordinary skill in the art of computer science. Moreover, it should be recognized that the stages could be implemented in hardware, firmware, software or any combination thereof.

The foregoing description, for purpose of explanation, has been described with reference to specific embodiments. However, the illustrative discussions above are not intended to be exhaustive or to be limiting to the precise forms disclosed. Many modifications and variations are possible in view of the above teachings. The embodiments were chosen and described in order to best explain the principles of the aspects and its practical applications, to thereby enable others skilled in the art to best utilize the aspects and various embodiments with various modifications as are suited to the particular use contemplated.

1.-15. (canceled)

16. A method of delivering radiation or particle beam therapy to a living subject's anatomy having physiological motion, the method comprising:
supporting the living subject by a programmable platform; and
determining an optimal counter motion strategy of the platform which results in one programmable movement from the group consisting of most tolerable, exact motion cancellation, most preferable by the subject, most preferable by a clinician, most preferable by an operator, most easily verifiable and safest movements.

17.-21. (canceled)

22. The method of claim 113,
wherein the radiation or particle beam therapy involves motion compensated imaging studies, such that the moving anatomy of interest appears to have diminished or no motion,
the method further comprising verifying adequate cancellation of the physiological motion prior to planning or delivering radiation or particle therapy by first conducting any of the motion compensated imaging studies.

23.-28. (canceled)

29. The method of claim 16, the method further comprising:
optimizing the counter-motion of the platform,
wherein a dosimetric treatment plan is generated for each motion strategy and the most desirable strategy is then chosen by one from the group consisting of clinician, subject, and operator, and
wherein appropriately minimized dosimetric planning margins are determined based on the chosen strategy.

30. (canceled)

31. A method of determining optimal counter motion strategies to reduce or cancel a physiological motion of a living subject's anatomy, the method comprising:
modeling the physiological motion patterns; and
determining a number of levels of counter motion of a programmable supporting platform under the living subject, wherein the levels comprise soft, moderate and extreme, corresponding to compromised reduction, significant reduction and complete cancelation of the physiological motion, respectively.

32.-35. (canceled)

36. The method of claim 31, further comprising having the living subject make deliberate changes to regular physiological motion.

37. (canceled)

38. The method of claim 31, further comprising verifying the effectiveness of the chosen counter motion strategy by imaging the subject on the same or similarly programmable supporting platform.

39. The method of claim 38, wherein imaging is conducted both with and without the chosen counter motion of the programmable supporting platform, and the two image sets are segmented and fused such that the moving anatomy of interest is imaged by the first set, whereas the nonmoving anatomy of the subject is imaged by the second set.

40.-45. (canceled)

46. The method of claim 16, wherein the programmable beam shaping method is a multileaf collimator (MLC).

47. (canceled)

48. The method of claim 46, wherein the moving target trajectories are further decomposed and allocated to appropriate subsystems based on the motion characteristics and conditions of the living subject, the said subsystems consisting of a programmable supporting platform, the MLC, and the carriages to which the MLC banks are separately attached.

49. The method of claim 48, wherein decomposition simplifies subsystem motions into separate orthogonal directions.

50. The method of claim 48, wherein decomposition takes into account low and high frequency components of the motion pattern, and thereafter allocates these different components according to the characteristics and optimal performance of the subsystems.

51. The method of claim 50, wherein the low frequency component of the motion pattern is allocated to the programmable supporting platform, resulting in more easily tolerated rocking motion that generates negligible voluntary/involuntary reactive movement by the living subject, and wherein the MLC subsystems further compensate the residual motion and high frequency variations.

52.-53. (canceled)

54. The method of claim 31, wherein, when soft or moderate tracking strategies are employed, any residual motion determined to be neglected by the platform motion is further compensated by shutting off or gating the radiation beam, wherein the radiation is paused for the brief duration wherein physiological motion excursion has exceeded the chosen range of moving platform compensation.

55. (canceled)

56. The method of claim 54 comprising coordinated compensation of physiological motion using one or more of the supporting platform, the beam shaping device and gating.

57.-71. (canceled)

72. A device for delivering radiation or particle beam therapy to a living subject's anatomy having physiological motion, the device comprising:
a programmable platform for supporting the living subject; and
a system for determining an optimal counter motion strategy of the platform which results in one programmable movement from the group consisting of most tolerable, exact motion cancellation, most preferable by the subject, most preferable by a clinician, most preferable by an operator, most easily verifiable and safest movements.

73.-86. (canceled)

87. A device for determining optimal counter motion strategies to reduce or cancel a physiological motion of a living subject's anatomy, the device comprising:
a system for modeling the physiological motion patterns; and
a system for determining a number of levels of counter motion of a programmable supporting platform under the living subject, wherein the levels comprise soft, moderate and extreme, corresponding to compromised reduction, significant reduction and complete cancelation of the physiological motion, respectively.

88.-91. (canceled)

92. The device of claim 87, further comprising a system adapted for having the living subject make deliberate changes to regular physiological motion.

93. (canceled)

94. The device of claim 87, further comprising a system for verifying the effectiveness of the chosen counter motion strategy by imaging the subject on the same or similarly programmable supporting platform.

95. The device of claim 94, wherein imaging is conducted both with and without the chosen counter motion of the programmable supporting platform, and the two image sets are segmented and fused such that the moving anatomy of interest is imaged by the first set, whereas the nonmoving anatomy of the subject is imaged by the second set.

96.-97. (canceled)

98. The device for modeling of claim 87, further comprising a first novel algorithm for improved prediction tumor motions for regular motion profiles and a second novel algorithm for improved prediction tumor motions for irregular motion profiles.

99. The device of claim 98, wherein each of the first and second novel algorithms comprises acceleration-enhanced artificial neural network (AE-ANN) effectively applicable for prediction of regular/normal motion of the tumor.

100. The device of claim 98, wherein each of the first and second novel algorithms comprises acceleration-enhanced normalized least mean squares (AE-nLMS) efficacious for predicting irregular/abnormal motion of the tumor.

101. (canceled)

102. The device of claim 72, wherein the programmable beam shaping device is a multileaf collimator (MLC).

103. (canceled)

104. The device of claim 102, wherein the moving target trajectories are further decomposed and allocated to appropriate subsystems based on the motion characteristics and conditions of the living subject, the said subsystems consisting of a programmable supporting platform, the MLC, and the carriages to which the MLC banks are separately attached to.

105. The device of claim 104, wherein decomposition simplifies subsystem motions into separate orthogonal directions, which can be for the purpose of easier tolerance by the living subject, or for the purpose of simpler operation/verification/safety or easier recording/capturing of the motion data.

106. The device of claim 104, wherein decomposition takes into account low and high frequency components of the motion pattern, and thereafter allocates these different components according to the characteristics and optimal performance of the subsystems.

107. The device of claim 106, wherein the low frequency component of the motion pattern is allocated to the programmable supporting platform, resulting in more easily tolerated rocking motion that generates negligible voluntary/involuntary reactive movement by the living subject, and wherein the MLC subsystems further compensate the residual motion and high frequency variations.

108.-109. (canceled)

110. The device of claim 87, wherein, when soft or moderate tracking strategies are employed, any residual motion determined to be neglected by the platform motion is further compensated by shutting off or gating the radiation beam, wherein the radiation is paused for the brief duration wherein physiological motion excursion has exceeded the chosen range of moving platform compensation.

111. The device of claim 72, comprising employment of both the moving platform and the beam shaping device together to compensate but not completely eliminate the physiological motion.

112. The device of claim 110 comprising coordinated compensation of physiological motion using one or more of the supporting platform, the beam shaping device and gating.

113. The method of claim 16, further comprising:
using a programmable beam shaping method to continuously conform the beam to a desired shape, such that any residual physiological motion uncanceled by the supporting platform is further canceled by a beam shaping device.

114. The method of claim 31, further comprising:
testing or training the living subject by the different levels of counter motion; and
choosing the most desirable level based on a trade off between preference, effectiveness in minimizing physiological motion, and minimization of voluntary or involuntary living subject movement in response to the motion of the programmable supporting platform.

115. The device of claim 72, further comprising:
a system for using a programmable beam shaping device to continuously conform the beam to a desired shape, such that any residual physiological motion uncanceled by the supporting platform is further canceled by a beam shaping device.

116. The device of claim 87, further comprising:
a system for testing or training the living subject by the different levels of counter motion; and
a system for choosing the most desirable level based on a trade off between preference, effectiveness in minimizing physiological motion, and minimization of voluntary or involuntary living subject movement in response to the motion of the programmable supporting platform.
